(19) United States
(12) Patent Application Publication
(10) Pub. No.: US 2012/0122180 A1
(43) Pub. Date: May 17, 2012

(54) INCORPORATION OF TYPE III POLYKETIDE SYNTHASES INTO MULTIDOMAIN PROTEINS OF THE TYPE I AND III POLYKETIDE SYNTHASE AND FATTY ACID SYNTHASE FAMILIES

(75) Inventors: Michael B. Austin, San Diego, CA (US); Joseph P. Noel, San Diego, CA (US); Marianne E. Bowman, San Diego, CA (US)

(73) Assignee: Salk Institute for Biological Studies, La Jolla, CA (US)

(21) Appl. No.: 13/294,939

(22) Filed: Nov. 11, 2011

Related U.S. Application Data
(63) Continuation of application No. 11/901,264, filed on Sep. 13, 2007, now abandoned.
(60) Provisional application No. 60/844,725, filed on Sep. 14, 2006.

(51) Int. Cl. C12N 9/96

(52) U.S. Cl. 435/188

(57) ABSTRACT

Recombinant fusion proteins in which intermediates are covalently bound to the fusion proteins and transferred between domains of the fusion proteins are provided. The fusion proteins include proteins having type I polyketide or fatty acid synthase domains fused with type III polyketide synthase domains. Methods of making such recombinant fusion proteins and methods using such proteins to produce polyketide and other products are described.

### CROSS-REFERENCE TO RELATED APPLICATIONS

This application is a non-provisional utility patent application claiming priority to and benefit of the following prior provisional patent application: U.S. Ser. No. 60/844,725, filed Sep. 14, 2006, entitled “INCORPORATION OF TYPE III POLYKETIDE SYNTHASES INTO MULTIDOMAIN PROTEINS OF THE TYPE I AND III POLYKETIDE SYNTHASE AND FATTY ACID SYNTHASE FAMILIES” by Michael B. Austin et al., which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant No. AI52443 from the National Institutes of Health. The government may have certain rights to this invention.

### FIELD OF THE INVENTION

The invention relates to recombinant fusion proteins in which intermediates are covalently bound to the fusion proteins. In particular, the invention relates to recombinant fusion proteins including type I polyketide or fatty acid synthase domains and type III polyketide synthase domains, methods of making such fusion proteins, and methods using such proteins to produce polyketide products.

### BACKGROUND OF THE INVENTION

Polyketides constitute an extensive class of structurally diverse compounds. Polyketides are synthesized by a broad range of naturally occurring organisms, including, for example, bacteria, marine organisms, fungi, and plants. They are typically produced by the stepwise condensation of simple carboxylic acid-derived starter and extender units in a set of reactions that closely parallels fatty acid biosynthesis. Polyketides achieve their structural diversity through this series of reactions, catalyzed by polyketide synthases, with features that contribute to diversity including the selection of various starter and extender units, final chain length, cyclization, degree of reduction, and the like. Downstream reactions such as glycosylation, hydroxylation, halogenation, prenylation, acylation, and alkylation can add additional diversity to the resulting products.

The extensive array of naturally occurring polyketides and their semisynthetic derivatives demonstrate an equally extensive range of activities. For example, a number of clinically effective drugs are based on polyketides, including antibiotics such as erythromycin and rifamycin, immunosuppressants such as rapamycin and FK506, antifungals such as amphotericin B, antiparasitics such as avermectin, insecticidals such as spinosyns, and anticancer agents such as doxorubicin, as just a few examples. Accordingly, polyketides are in high demand as lead compounds for drug discovery.

Ability to synthesize polyketides, whether to more conveniently produce large quantities of known polyketides or to produce novel polyketides, is thus highly desirable. Among other aspects, the present invention provides methods for polyketide synthesis. A complete understanding of the invention will be obtained upon review of the following.

### SUMMARY OF THE INVENTION

One aspect of the invention provides recombinant fusion proteins in which intermediates are covalently bound to the fusion proteins and transferred between domains of the fusion proteins, including proteins having type I polyketide or fatty acid synthase domains fused with type III polyketide synthase domains. Other aspects of the invention provide methods of making such recombinant fusion proteins and methods using such proteins to produce polyketides and other products.

One general class of embodiments provides a recombinant fusion protein that comprises at least one type I polyketide synthase (PKS) domain or type I fatty acid synthase (FAS) domain and a type III polyketide synthase domain. Typically, the at least one type I polyketide or fatty acid synthase domain catalyzes conversion of one or more first precursors to an intermediate which is covalently bound to the fusion protein, and the type III PKS domain catalyzes conversion of the intermediate to a polyketide product.

The at least one type I polyketide or fatty acid synthase domain typically comprises one or more of a ketoacyl synthase domain, an acyl transferase domain, a dehydratase domain, an enoyl reductase domain, a ketoreductase domain, and an acyl carrier domain. The fusion protein optionally includes two or more, three or more, four or more, five or more, or even six or more such domains. For example, in one class of embodiments, the recombinant fusion protein includes type I fatty acid synthase ketoacyl synthase, acyl transferase, dehydratase, enoyl reductase, ketoreductase, and acyl carrier domains.

The recombinant fusion protein optionally includes a type III PKS domain derived from a protein including, but not limited to, chalcone synthase, stilbene synthase, stilbenecarboxylate synthase, bibenzyl synthase, homoeriodictyol/eriodictyol synthase, acridone synthase, benzophenone synthase, phlorisovalerophenone synthase, coumaroyl triacetic acid synthase, benzalacetone synthase, 1,3,6,8-tetrahydroxynaphthalene synthase, phloroglucinol synthase, dihydroxyphenylacetate synthase, alkylresorcinol synthase, alkylpyrone synthase, aloesone synthase, pentaketide chromone synthase, octaketide synthase, the Steely2 C-terminal domain, and benzalacetone synthase. The type III polyketide synthase domain is optionally C-terminal to the at least one type I polyketide synthase domain or type I fatty acid synthase domain in the recombinant fusion protein.

The recombinant fusion protein optionally includes one or more domains derived from the Steely1 or Steely2 proteins described herein (SEQ ID NO:1 and 2, respectively). For example, the fusion protein optionally includes one or more of a ketoacyl synthase domain, acyl transferase domain, dehydratase domain, enoyl reductase domain, ketoreductase domain, and acyl carrier domain derived from Steely1 or Steely2. In one class of embodiments, the fusion protein includes the Steely1 PKS III domain (approximately residues 2776-3147 of SEQ ID NO:1); the Steely1 PKS III domain and the linker N-terminal to it (approximately residues 2629-3147 of SEQ ID NO:1); the Steely1 AC domain, PKS III domain, and the linker connecting them (approximately residues 2560-3147 of SEQ ID NO:1); or the Steely1 linker connecting the AC and PKS III domains (approximately residues 2629-2775 of SEQ ID NO:1); or an amino acid sequence at least about 90% identical thereto. In another class of embodiments, the fusion protein includes the Steely2 PKS III domain (approximately residues 2616-2968 of SEQ ID NO:2); the Steely2 PKS III domain and the linker N-terminal to it (approximately residues 2473-2968 of SEQ ID NO:2); the Steely2 AC domain, PKS III domain, and the linker connecting them (approximately residues 2412-2968 of SEQ ID NO:2); or the Steely2 linker connecting the AC and PKS III domains (approximately residues 2473-2615 of SEQ ID NO:2); or an amino acid sequence at least about 90% identical thereto.

Another general class of embodiments provides a recombinant fusion protein that comprises at least a first domain that catalyzes conversion of one or more precursors to an intermediate, which intermediate is covalently bound to the fusion protein, and a second domain that catalyzes conversion of the intermediate to a product. The product is typically released by the second domain.

The first and second domains used to create the recombinant fusion protein are derived from different parental polypeptides. Typically, the first and second polypeptide are enzymes of different types or belonging to different families. For example, when the first domain is a type I PKS domain, the second domain is other than a type I PKS domain. Similarly, when the first domain is a non-ribosomal peptide synthetase (NRPS) domain, the second domain is other than an NRPS domain. Optionally, when the at least one first domain comprises a type I PKS domain or an NRPS domain, the second domain is other than a type I PKS domain or an NRPS domain.

In one class of embodiments, the product is released by the second domain, and the second domain is other than a thioesterase domain. The second domain optionally replaces a thioesterase domain (or another product-releasing domain) in a first enzyme from which the first domain is derived. The second domain is optionally C-terminal to the first domain.

In one class of embodiments, the first domain is derived from an enzyme that catalyzes conversion of the one or more precursors to a diffusible product. For example, the first domain can be derived from a type I FAS, a type I PKS, a non-ribosomal peptide synthetase (NRPS), or a mixed NRPS/PKS. While the parental enzyme releases a diffusible product, in the context of the recombinant fusion protein, the domain derived from the enzyme produces a covalently bound moiety.

In one class of embodiments, the second domain is derived from an enzyme that catalyzes conversion of a diffusible substrate to product. While the parental enzyme acts on a diffusible substrate, in the context of the recombinant fusion protein, the domain derived from the enzyme acts on a covalently bound substrate (the intermediate that results from the action of the first domain). For example, in one class of embodiments, the fusion protein comprises an acyl carrier domain to which the intermediate is covalently bound, and the second domain is selected from the group consisting of: a beta-ketosynthase domain, an aromatic iterative polyketide synthase domain, a type III polyketide synthase domain, a type II polyketide synthase domain, a non-iterative polyketide synthase domain, an HMG-CoA synthetase domain, a ketoacyl-synthase III domain, and a beta-ketoacyl CoA synthase domain.

One class of embodiments provides a recombinant fusion protein wherein the first domain is a type I polyketide synthase domain or type I fatty acid synthase domain and wherein the fusion protein comprises an acyl carrier domain to which the intermediate is covalently bound. The second domain is optionally a type III polyketide synthase domain, by which the product is released.

In one aspect, the invention provides methods of making a fusion protein. In the methods, one or more first DNA molecules collectively encoding one or more type I polyketide synthase or fatty acid synthase domains are provided. At least one second DNA molecule encoding a type III polyketide synthase domain is also provided. The one or more first DNA molecules are joined in frame with the second DNA molecule to generate a recombinant DNA molecule encoding the fusion protein, then the recombinant DNA molecule is translated to produce the fusion protein.

Libraries of recombinant DNA molecules are optionally produced and screened to identify fusion proteins(s) possessing a desired activity (e.g., use of a particular precursor and/or production of a particular product). Thus, in one embodiment, providing one or more first DNA molecules comprises providing a library of first DNA molecules differing from each other in at least one nucleotide. In a related embodiment, providing at least one second DNA molecule comprises providing a library of second DNA molecules differing from each other in at least one nucleotide. In one class of embodiments, joining the one or more first DNA molecules with the second DNA molecule to generate a recombinant DNA molecule comprises joining one or more first DNA molecules or a library thereof with the second DNA molecule or a library thereof to generate a library of recombinant DNA molecules. The library of recombinant DNA molecules can then be translated to provide a library of fusion proteins, which is screened for a desired property. A library of first DNA molecules, a library of second DNA molecules, and/or the library of recombinant DNA molecules is optionally subjected to DNA shuffling.

The fusion proteins of the invention can be used to produce products. Accordingly, one aspect of the invention provides methods of making a polyketide product. In the methods, a recombinant fusion protein comprising at least one type I polyketide synthase or type I fatty acid synthase domain and a type III polyketide synthase domain is provided. One or more first precursors are contacted with the recombinant fusion protein, whereby the at least one type I polyketide synthase or fatty acid synthase domain catalyzes conversion of the one or more first precursors to an intermediate, and the type III polyketide synthase domain catalyzes conversion of the intermediate (and optionally one or more second precursors) to the polyketide product. Typically, the intermediate is covalently bound to the fusion protein. In one class of embodiments, the first precursors and the recombinant fusion protein are contacted inside a cell expressing the recombinant fusion protein.

The product can be any of an extremely wide variety of polyketones. As just a few examples, the product can be an aliphatic methylketone, a phloroglucinol, an acyl phloroglucinol, a branched acyl phloroglucinol, a phlorisovalerophenone, a chalcone, an acridone, a bibenzyl, an acyl resorcinol, an acyl resorcinolic acid, an alkyl resorcinol, a stilbene, a stilbene acid, a tetrahydroxynaphthalene, an acyl chromone, an acyl lactone, an acyl pyrone, an olivetol, or an olivitolic acid product.

The recombinant fusion protein can be any of those described herein. For example, the fusion protein can include one or more of a ketoacyl synthase domain, an acyl transferase domain, a dehydratase domain, an enoyl reductase domain, a ketoreductase domain, and an acyl carrier domain. In one class of embodiments, the recombinant fusion protein includes type I fatty acid synthase ketoacyl synthase, acyl transferase, dehydratase, enoyl reductase, ketoreductase, and acyl carrier domains. The recombinant fusion protein optionally includes a type III PKS domain derived from a protein including, but not limited to, chalcone synthase, stilbene synthase, stilbenecarboxylate synthase, bibenzyl synthase, homoeriodictyol/eriodictyol synthase, acridone synthase, benzophenone synthase, phlorisovalerophenone synthase, coumaroyl triacetic acid synthase, benzalacetone synthase, 1,3,6,8-tetrahydroxynaphthalene synthase, phloroglucinol synthase, dihydroxyphenylacetate synthase, alkylresorcinol synthase, alkylpyrone synthase, aloesone synthase, pentaketide chromone synthase, octaketide synthase, the Steely2 C-terminal domain, and benzalacetone synthase. The type III polyketide synthase domain is optionally C-terminal to the at least one type I polyketide synthase domain or type I fatty acid synthase domain in the recombinant fusion protein.

The recombinant fusion protein optionally includes one or more domains derived from the Steely1 or Steely2 proteins described herein (SEQ ID NO:1 and 2, respectively). For example, the fusion protein optionally includes the Steely1 PKS III domain (approximately residues 2776-3147 of SEQ ID NO:1); the Steely1 PKS III domain and the linker N-terminal to it (approximately residues 2629-3147 of SEQ ID NO:1); the Steely1 AC domain, PKS III domain, and the linker connecting them (approximately residues 2560-3147 of SEQ ID NO:1); or the Steely1 linker connecting the AC and PKS III domains (approximately residues 2629-2775 of SEQ ID NO:1); or an amino acid sequence at least about 90% identical thereto. In another class of embodiments, the fusion protein includes the Steely2 PKS III domain (approximately residues 2616-2968 of SEQ ID NO:2); the Steely2 PKS III domain and the linker N-terminal to it (approximately residues 2473-2968 of SEQ ID NO:2); the Steely2 AC domain, PKS III domain, and the linker connecting them (approximately residues 2412-2968 of SEQ ID NO:2); or the Steely2 linker connecting the AC and PKS III domains (approximately residues 2473-2615 of SEQ ID NO:2); or an amino acid sequence at least about 90% identical thereto.

In one aspect, the invention provides a variety of polynucleotides encoding the fusion proteins of the invention. For example, one class of embodiments provides an expression vector that includes a promoter operably linked to a polynucleotide encoding a fusion protein that comprises at least one type I polyketide or fatty acid synthase domain and a type III polyketide synthase domain. The protein is optionally a recombinant fusion protein. A related class of embodiments provides a cell comprising such an expression vector. The cell optionally expresses one or more enzymes whose collective action converts a polyketide product of the fusion protein into a final product. Such downstream tailoring enzymes can perform glycosylation, hydroxylation, halogenation, prenylation, acylation, alkylation, oxidation, and/or similar steps as necessary to produce the desired final product.

The fusion protein can be any of those described herein. For example, the fusion protein can include one or more of a ketoacyl synthase domain, an acyl transferase domain, a dehydratase domain, an enoyl reductase domain, a ketoreductase domain, and an acyl carrier domain. In one class of embodiments, the recombinant fusion protein includes type I fatty acid synthase ketoacyl synthase, acyl transferase, dehydratase, enoyl reductase, ketoreductase, and acyl carrier domains. The recombinant fusion protein optionally includes a type III PKS domain derived from a protein including, but not limited to, chalcone synthase, stilbene synthase, stilbenecarboxylate synthase, bibenzyl synthase, homoeriodictyol/eriodictyol synthase, acridone synthase, benzophenone synthase, phlorisovalerophenone synthase, coumaroyl triacetic acid synthase, benzalacetone synthase, 1,3,6,8-tetrahydroxynaphthalene synthase, phloroglucinol synthase, dihydroxyphenylacetate synthase, alkylresorcinol synthase, alkylpyrone synthase, aloesone synthase, pentaketide chromone synthase, octaketide synthase, the Steely2 C-terminal domain, and benzalacetone synthase. The type III polyketide synthase domain is optionally C-terminal to the at least one type I polyketide synthase domain or type I fatty acid synthase domain in the recombinant fusion protein.

The fusion protein optionally includes one or more domains derived from the Steely1 or Steely2 proteins described herein (SEQ ID NO:1 and 2, respectively). For example, the fusion protein optionally includes the Steely1 PKS III domain (approximately residues 2776-3147 of SEQ ID NO:1); the Steely1 PKS III domain and the linker N-terminal to it (approximately residues 2629-3147 of SEQ ID NO:1); the Steely1 AC domain, PKS III domain, and the linker connecting them (approximately residues 2560-3147 of SEQ ID NO:1); or the Steely1 linker connecting the AC and PKS III domains (approximately residues 2629-2775 of SEQ ID NO:1); or an amino acid sequence at least about 90% identical thereto. In another class of embodiments, the fusion protein includes the Steely2 PKS III domain (approximately residues 2616-2968 of SEQ ID NO:2); the Steely2 PKS III domain and the linker N-terminal to it (approximately residues 2473-2968 of SEQ ID NO:2); the Steely2 AC domain, PKS III domain, and the linker connecting them (approximately residues 2412-2968 of SEQ ID NO:2); or the Steely2 linker connecting the AC and PKS III domains (approximately residues 2473-2615 of SEQ ID NO:2); or an amino acid sequence at least about 90% identical thereto. Optionally, the fusion protein includes 50 or more contiguous amino acids of SEQ ID NO:1 or SEQ ID NO:2 (e.g., 100 or more, 200 or more, 300 or more, 400 or more, 500 or more, 1000 or more, 1500 or more, 2000 or more, or even 2500 or more), or an amino acid sequence at least about 25% identical thereto (e.g., at least about 50%, at least about 75%, at least about 90%, at least about 95%, at least about 97%, or at least about 99% identical thereto).

### BRIEF DESCRIPTION OF THE DRAWINGS

FIG. 1 Panel A is a schematic illustration of DIF-1 synthesis using previously available information, showing that phlorocaprophenone (PCP) is an intermediate in the biosynthesis of DIF-1. Panel B illustrates exemplary substrate and product diversity of reactions catalyzed by iterative CHS-like enzymes. Panel C schematically illustrates proposed PCP biosynthesis by a steely FAS I-PKS III hybrid. Direct transfer of a hexanoyl intermediate to the type III PKS domain is based on analogous off loading of conventional type I FAS/PKS products via activity of thioesterase (TE) domains, as shown in Panel D. Panel D schematically illustrates that in metazoan type I FASs and related type I PKSs a C-terminal thioesterase (TE) domain catalyzes the hydrolytic release of enzymatic products from the prosthetic phosphopantetheine arm of the adjacent acyl carrier protein (ACP) domain.

FIG. 2 schematically illustrates the domain structures of the novel D. discoideum fusion proteins Steely1 (DDB0190208) and Steely2 (DDB0219613).

FIG. 3 presents a sequence alignment of the Steely1 and Steely2 C-terminal domains (residues 2776-3147 of SEQ ID NO:1 and residues 2595-2968 of SEQ ID NO:2, respectively) with alfalfa CHS (SEQ ID NO:5). Asterisks mark positions of the type III PKS Cys-His-Asn catalytic triad. The alignment was produced using multalin (available at prodes (dot) toulouse (dot) inra (dot) fr/multalin/; see Corpet (1988) “Multiple sequence alignment with hierarchical clustering” Nucl. Acids Res. 16:10881-10890) using the default setting using Blosum62-12-2 alignment tables (Henikoff and Henikoff (1992) “Amino acid substitution matrices from protein blocks” Proc Natl Acad Sci USA 89:10915-10919). In the consensus sequence (SEQ ID NOs:6-13), red uppercase indicates high consensus residues and blue lowercase indicates low consensus residues; black is neutral. A position with no conserved residue is represented by a dot in the consensus line, and ! is any one of IV, $ is any one of LM, % is any one of FY, and # is any one of NDQEBZ.

FIG. 4 depicts the FAS-like N-terminal sequences of Steely1 and Steely2, showing a sequence alignment of the first six N-terminal Steely domains (residues 1-2775 of SEQ ID NO:1 and residues 1-2594 of SEQ ID NO:2) with the first six N-terminal domains of human FAS (SEQ ID NO:14), as well as the full-length sequences of two related D. discoideum ORFs (SEQ ID NOs:15-16). The alignment was generated as and symbols are as in FIG. 3. The consensus sequence is listed as SEQ ID NOs:17-65.

FIG. 5 illustrates polyketide extension of various acyl-CoA substrates by the heterologously expressed C-terminal domains of Steely1 and Steely2. An autoradiogram of thin layer chromatography analysis of in vitro assays using 14-C labeled malonyl-CoA and one of five acyl substrates is shown on the right; the substrates are depicted on the left. Substrate 1 is the physiological substrate of CHS, while substrate 3 is the starter used for type III PKS production of phlorocaprophenone.

FIG. 6 illustrates hexanoyl-primed in vitro product specificity of steely C-terminal type III PKS domains. Panel A illustrates polyketide cyclization routes leading to acylpyrones (blue arrows) and acylphloroglucinols (red arrows). Carbons 1, 5, and 6 are involved in cyclization. Sphere represents CoA or active site cysteine. Starter-derived moieties are green and circled with a dashed line; n=3 and n=2 for hexanoyl and pentanoyl moieties (respectively) of known D. discoideum acylphloroglucinols, and n=3 and n=1 for hexanoyl- and butanoyl-CoA substrates (respectively) tested here (see Panel B and FIGS. 7 and 8). Conversely, dictyopyrone biosynthesis may involve condensation of a diketide (black) with another small molecule (gold and circled). Panel B illustrates acylphloroglucinol (PCP) biosynthesis by Steely2 but not Steely1. Main enzymatic products of hexanoyl-CoA-primed in vitro type III PKS assays with malonyl-CoA as determined by negative-mode LC-MS-MS (insets). Parent (MS) masses for each MS-MS spectrum are given in blue parentheses.

FIG. 7 illustrates LC-MS-MS analysis of all hexanoyl-primed products of in vitro enzyme assays with malonyl-CoA, for Panel A Steely1 type III PKS domain, Panel B Steely2 type III PKS domain, Panel C synthetic phlorocaprophenone (PCP) authentic standard, and Panel D alfalfa CHS. In all panels, arrows on the upper UV (286 nm) chromatograms identify enzymatic or standard product peaks analyzed using negative ion MS-MS mass spectra, displayed as insets on lower extracted ion chromatograms (EICs). Blue and green EIC traces track masses consistent with hexanoyl-primed tri- and tetra-ketide products, as indicated. Parent (MS) masses for each MS-MS analysis are given in blue parentheses. Product identification is based upon comparison with authentic PCP standard and published LC-MS-MS analyses of hexanoyl-derived tri- and tetra-ketide acyl pyrone and acyl phloroglucinol synthetic standards, as well as comparison with the known hexanoyl-primed in vitro products of alfalfa CHS.

FIG. 8 illustrates LC-MS-MS analysis of all butanoyl-primed products of in vitro enzyme assays with malonyl-CoA. Panel A illustrates butanoyl-primed major products of steely C-terminal domains and alfalfa CHS, displayed in the manner of FIG. 6 Panel B. Inset mass spectra represent negative MS-MS of the largest UV absorbance (at 286 nm) peaks. Parent (MS) masses for each MS-MS spectrum are given in blue parentheses. Panels B-D illustrate complete UV traces and negative ion LCMS-MS analyses of all butanoyl-primed tri- and tetraketide enzymatic products of Panel B Steely1 type III PKS domain, Panel C Steely2 type III PKS domain, and Panel D alfalfa CHS. Arrows on upper UV (286 nm) chromatograms identify product peaks analyzed using negative ion MS-MS mass spectra, displayed as insets on lower extracted ion chromatograms (EICs). Blue and green EIC traces track masses consistent with tri- and tetra-ketide products, as indicated. Parent (MS) masses for each MS-MS analysis are given in parentheses. Product identification is based upon relative retention times, parent ion masses, and negative ion LC-MS-MS fragmentation patterns analogous to those observed for hexanoyl-derived products.

FIG. 9 illustrates results from crystallographic analysis of the Steely1 C-terminal CHS-like domain. Panel A depicts a ribbon diagram overlay of D. discoideum Steely1 C-terminal domain homodimer (cyan and copper) with that of alfalfa CHS (grey). Superimposed CHS complexed ligands in gold (CoA and naringenin from different crystal structures) illustrate CoA binding site and internal active site cavity. A molecule of PEG serendipitously bound in the active site entrance of Steely1 is shown in CPK violet and red. Panel B depicts a closer view of the superimposed Steely1 and CHS active sites, using the same color scheme, showing conservation of the catalytic triad and confirming homology-predicted assignments of important active site residues but with subtle conformational changes. Note interaction of PEG with the His-Asn oxyanion hole. Panel C depicts a similar view of a homology model of the Steely2 C-terminal domain (lavender) overlaid with the Steely1 crystal structure. Note that some variation of active site residues is observed.

Schematic figures are not necessarily to scale.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

The term “about” as used herein indicates the value of a given quantity varies by +/−10% of the value, or optionally +/−1-5% of the value, or in some embodiments, by +/−1% of the value so described.

The term “recombinant” indicates that the material (e.g., a nucleic acid or a protein) has been artificially or synthetically (non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid.

The term “fusion protein” indicates that the protein includes polypeptide components derived from more than one parental protein or polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a cell as a single protein.

A “domain” of a protein is any portion of the entire protein, up to and including the complete protein but typically comprising less than the complete protein. A domain can, but need not, fold independently of the rest of the protein chain and/or be correlated with a particular biological function or location (e.g., an enzymatic activity, attachment site of a prosthetic group, etc.).

As used herein, the term “derived from” refers to a component that is isolated from or made using a specified molecule or organism, or information from the specified molecule or organism. For example, a polypeptide that is derived from a second polypeptide comprises an amino acid sequence that is identical or substantially similar (or substantially identical) to an amino acid sequence of the second polypeptide. In the case of polypeptides, the derived species can be obtained by, for example, naturally occurring mutagenesis, artificial directed mutagenesis, or artificial random mutagenesis. The mutagenesis used to derive polypeptides can be intentionally directed or intentionally random. The mutagenesis of a polypeptide to create a different polypeptide derived from the first can be a random event (e.g., caused by polymerase infidelity) and the identification of the derived polypeptide can be serendipitous or purposeful. Mutagenesis of a polypeptide typically entails manipulation of the polynucleotide that encodes the polypeptide. A domain “derived from” a specified protein, e.g., a multidomain protein, is typically isolated from its usual context in that protein (for example, any flanking domains and/or other amino acid sequences are deleted) and is optionally placed in a different context (for example, flanked by one or more domains and/or other amino acid sequences derived from a different protein, to form a fusion protein); the domain optionally includes additional mutations (e.g., amino acid substitutions or insertions) as compared to the parental protein from which it was derived.

“Type I fatty acid synthases” include known and/or naturally occurring type I fatty acid synthases, as well as polypeptides homologous thereto and/or derived therefrom and exhibiting one or more enzymatic activities characteristic of such fatty acid synthases.

A “type I fatty acid synthase domain” is a domain derived from a type I fatty acid synthase. The type I fatty acid synthase can be, for example, a naturally occurring fatty acid synthase or a recombinant fatty acid synthase, e.g., produced by mutagenesis, recombination of domains, DNA shuffling, or similar techniques.

“Type I polyketide synthases” include known and/or naturally occurring type I polyketide synthases, as well as polypeptides homologous thereto and/or derived therefrom and exhibiting one or more enzymatic activities characteristic of such polyketide synthases.

A “type I polyketide synthase domain” is a domain derived from a type I polyketide synthase. The type I polyketide synthase can be, for example, a naturally occurring polyketide synthase or a recombinant polyketide synthase, e.g., produced by mutagenesis, recombination of domains, DNA shuffling, or similar techniques.

“Type III polyketide synthases” include known and/or naturally occurring type III polyketide synthases, as well as polypeptides homologous thereto and/or derived therefrom and exhibiting one or more enzymatic activities characteristic of such polyketide synthases.

A “type III polyketide synthase domain” is a domain derived from a type III polyketide synthase. The type III polyketide synthase can be, for example, a naturally occurring polyketide synthase or a recombinant polyketide synthase, e.g., produced by mutagenesis, recombination of domains, DNA shuffling, or similar techniques.

A “polypeptide” is a polymer comprising two or more amino acid residues (e.g., a peptide or a protein). The polymer can additionally comprise non-amino acid elements such as labels, quenchers, blocking groups, or the like and can optionally comprise modifications such as glycosylation or the like. The amino acid residues of the polypeptide can be natural or non-natural and can be unsubstituted, unmodified, substituted or modified.

An “amino acid sequence” or “polypeptide sequence” is a polymer of amino acid residues (a protein, polypeptide, etc.) or a character string representing an amino acid polymer, depending on context.

The term “nucleic acid” or “polynucleotide” encompasses any physical string of monomer units that can be corresponded to a string of nucleotides, including a polymer of nucleotides (e.g., a typical DNA or RNA polymer), PNAs, modified oligonucleotides (e.g., oligonucleotides comprising nucleotides that are not typical to biological RNA or DNA, such as 2′-O-methylated oligonucleotides), and the like. A nucleic acid can be e.g., single-stranded or double-stranded. Unless otherwise indicated, a particular nucleic acid sequence of this invention encompasses complementary sequences, in addition to the sequence explicitly indicated.

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

“Expression of a gene” or “expression of a nucleic acid” means transcription of DNA into RNA (optionally including modification of the RNA, e.g., splicing), translation of RNA into a polypeptide (possibly including subsequent modification of the polypeptide, e.g., posttranslational modification), or both transcription and translation, as indicated by the context.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not autonomously replicating. Most commonly, the vectors of the present invention are plasmids.

An “expression vector” is a vector, such as a plasmid, which is capable of promoting expression as well as replication of a nucleic acid incorporated therein. Typically, the nucleic acid to be expressed is “operably linked” to a promoter and/or enhancer, and is subject to transcription regulatory control by the promoter and/or enhancer.

As used herein, the term “encode” refers to any process whereby the information in a polymeric macromolecule or sequence string is used to direct the production of a second molecule or sequence string that is different from the first molecule or sequence string. As used herein, the term is used broadly, and can have a variety of applications. In one aspect, the term “encode” describes the process of semi-conservative DNA replication, where one strand of a double-stranded DNA molecule is used as a template to encode a newly synthesized complementary sister strand by a DNA-dependent DNA polymerase. In another aspect, the term “encode” refers to any process whereby the information in one molecule is used to direct the production of a second molecule that has a different chemical nature from the first molecule. For example, a DNA molecule can encode an RNA molecule (e.g., by the process of transcription incorporating a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

The term “introduced” when referring to a heterologous or isolated nucleic acid refers to the transfer of a nucleic acid into a eukaryotic or prokaryotic cell where the nucleic acid can be incorporated into the genome of the cell (e.g., chromosome, plasmid, plastid or mitochondrial DNA), converted into an autonomous replicon, or transiently expressed (e.g., transfected mRNA). The term includes such methods as “infection,” “transfection,” “transformation” and “transduction.” In the context of the invention a variety of methods can be employed to introduce nucleic acids into host cells, including electroporation, calcium phosphate precipitation, lipid mediated transfection (lipofection), biolistic delivery, etc.

The term “host cell” means a cell which contains a heterologous nucleic acid, such as a vector, and supports the replication and/or expression of the nucleic acid. Host cells can be prokaryotic cells such as E. coli, or eukaryotic cells such as yeast, plant, insect, amphibian, avian, or mammalian cells, including human cells.

A “promoter”, as used herein, includes reference to a region of DNA upstream from the start of transcription and involved in recognition and binding of RNA polymerase and other proteins to initiate transcription. An “inducible” promoter is a promoter that is under environmental control and may be inducible or de-repressible. Examples of environmental conditions that may effect transcription by inducible promoters include exposure to a particular chemical, anaerobic conditions, or the presence of light. Tissue-specific, cell-type-specific, and inducible promoters constitute the class of “non-constitutive”promoters. A “constitutive” promoter is a promoter that is active under most environmental conditions and, if applicable, in all or nearly all tissues at all or nearly all stages of development.

A variety of additional terms are defined or otherwise characterized herein.

### DETAILED DESCRIPTION

As described above, polyketides can be produced in a series of reactions catalyzed by polyketide synthases. These enzymes can be manipulated to control the nature of the resulting polyketide products. Among other aspects, the present invention provides novel enzymes that can catalyze production of polyketides. The enzymes include one or more type I polyketide synthase or fatty acid synthase domains fused with at least one type III polyketide synthase domain. Additional fusion proteins are also provided. Methods of making such fusion proteins, compositions useful in making such fusion proteins, and methods of making polyketides or other products using such fusion proteins are also described.

While a brief overview of Fatty Acid Synthase (FAS) and Polyketide Synthase (PKS) background information is provided below, a few useful reviews provide further and comprehensive background information as well as specific experimental references. With some overlap, these comprehensive reviews focus on FAS systems (Rawlings (1998) “Biosynthesis of fatty acids and related metabolites” Nat Prod Rep 15(3):275-308), Type I PKS systems (Staunton and Weissman (2001) “Polyketide biosynthesis: a millennium review” Nat Prod Rep 18(4):380-416), and the type III PKS superfamily (Austin and Noel (2003) “The chalcone synthase superfamily of type III polyketide synthases” Nat Prod Rep 20:79-110). Type I FAS structural models (featuring monomeric TE domains) are discussed in two more recent papers (Chirala and Wakil (2004) “Structure and function of animal fatty acid synthase” Lipids 39(11):1045-53 and Rangan et al (2001) “Mapping the functional topology of the animal fatty acid synthase by mutant complementation in vitro” Biochemistry” 40(36):10792-9), and the crystal structure of a homodimeric type I PKS TE is also available (Tsai et al. (2001) “Crystal structure of the macrocycle-forming thioesterase domain of the erythromycin polyketide synthase: versatility from a unique substrate channel” Proc Natl Acad Sci USA 98(26):14808-13). Recent results relevant to FAS and type I PKS structural models can also be found in Maier et al. (2006) “Architecture of mammalian fatty acid synthase at 4.5 A resolution” Science 311(5765):1258-62, Tang et al. (2006) “The 2.7-Angstrom crystal structure of a 194-kDa homodimeric fragment of the 6-deoxyerythronolide B synthase” Proc Natl Acad Sci USA. 103(30):11124-9, and Tang et al. (2007) “Structural and mechanistic analysis of protein interactions in module 3 of the 6-deoxyerythronolide B synthase” Chem. Biol. 14(8):931-43. Efforts toward control and combinatorial engineering of type I PKS systems (Menzella et al. (2005) “Combinatorial polyketide biosynthesis by de novo design and rearrangement of modular polyketide synthase genes” Nat Biotechnol 23:1171-1176), as well as structural characterization of their domain linkage interactions (Broadhurst et al. (2003) “The structure of docking domains in modular polyketide synthases” Chem Biol 10:723-731), have yielded recent results, as summarized succinctly in a related article (Sherman (2005) “The Lego-ization of polyketide biosynthesis” Nat Biotechnol 23(9):1083-1084). A brief introduction to Dictyostelium discoideum and a detailed description of the bioinformatic discovery and experimental study of naturally occurring type I FAS/PKS-type III PKS fusion proteins, the Steely enzymes, are presented in Example 1 herein.

### Type I Fatty Acid and Polyketide Synthases

Type I FAS enzymes are multi-domain polypeptides whose various domains catalyze the activities associated with fatty acid biosynthesis, each cycle of which adds two carbons to the aliphatic tail of a thioester-linked fatty acyl starter molecule. FAS systems complete each cycle by catalyzing one condensation and three reduction steps, with the help of a small handful of ancillary activities and protein domains. Substrates and intermediate products are typically maintained as thioester conjugates to one of two carrier molecules: either the small molecule coenzyme A (CoA) or the FAS acyl carrier protein (ACP) domain. Both carrier molecules utilize the same phosphopantetheine prosthetic group, whose terminal thiol participates in the thioester bond with the acyl substrate. Thioester bonds are utilized because they are weaker than similar bonds to carbon or oxygen. Their relatively high-energy state allows for facile isoenergetic transfer of substrates to catalytically essential active site cysteines, as well as energetically favorable formation of carbon-carbon bonds.

While short chain acyl-CoAs such as acetyl-CoA are common end products of various degradative pathways, ACP is the preferred carrier for most FAS biosynthetic enzymes. Substrates must typically thus first be activated by transfer to an ACP by an acyltransferase (AT) activity, sometimes called malonyl acyltransferase (MAT) to reflect its additional role in the transfer of the malonyl extender unit to ACP, whereupon it is used for polyketide chain extension. Following the transfer of the substrate to the ketoacyl synthase (KAS or KS) domain's catalytic cysteine, this condensing enzyme catalyzes the addition of a two-carbon acetate unit to the enzyme bound thioester end of the fatty acid, via a decarboxylative condensation with malonyl-ACP. The resulting ACP-bound β-ketoacyl thioester is presented to an NADPH-dependent β-ketoacyl-ACP reductase (KR), which reduces the original substrate carbonyl (now the β-keto carbonyl) to an alcohol. A β-hydroxyacyl dehydratase (DH) catalyzes loss of water, leaving a carbon-carbon double bond. An NADH-dependent enoyl-ACP reductase (ER) module completes the reduction of the β-carbon, resulting in an acyl-ACP that resembles the original substrate, but with two additional methylene moieties. Type I FAS enzymes are typically iterative, performing several cycles of elongation before their terminal thioesterase (TE) domain releases the product as a free fatty acid. In vivo, it can be difficult to assess whether the final product length specificity of a FAS system depends more upon its thioesterase or its KS domains.

Type I FAS systems typically include the above activities (ACP, AT, KS, KR, DH, ER, and TE) in distinct domains on one or two multi-functional, multi-domain protein chains. For example, mammalian FAS activities are typically encoded in a single polypeptide that functions as a homodimer (Rangan et al. (2001) “Mapping the functional topology of the animal fatty acid synthase by mutant complementation in vitro” Biochemistry 40:10792-10799 and Maier et al. (2006) “Architecture of mammalian fatty acid synthase at 4.5 A resolution” Science 311(5765):1258-62), while yeast FAS activities are typically distributed across two polypeptide chains that function as a multimeric complex (Rawlings (1998) “Biosynthesis of fatty acids and related metabolites” Nat Prod Rep 15:275-308 and Jenni et al. (2006) “Architecture of a fungal fatty acid synthase at 5 Å resolution” Science 311(5765):1263-7).

Like FAS systems, PKS systems include a β-keto synthase (KS) activity that catalyzes the sequential head-to-tail incorporation of two-carbon acetate units into a growing polyketide chain. However, whereas FAS systems perform reduction and dehydration reactions on each resulting β-keto carbon to produce an inert hydrocarbon, PKS systems omit or modify some of these latter reactions, thus preserving varying degrees of polar chemical reactivity along portions of the growing linear polyketide chain. Various PKS enzymes selectively exploit the reactivity of polyketide intermediates to promote intramolecular cyclization and π-bond rearrangement, generating an amazingly diverse collection of substituted monocyclic and polycyclic products from a simple acetyl building block.

Domains of type I PKS enzymes generally retain the genetic domain organization found in type I FAS enzymes, but some or all of the domains catalyzing reduction and dehydration are catalytically inactive or in some cases altogether missing. Type I PKS systems can be either iterative, like typical type I FAS systems, or modular, with each FAS-like module of domains catalyzing a single round of polyketide extension (with or without subsequent β-keto reduction and dehydration). The first module of a modular type I PKS systems often contains an AT domain, responsible for starter molecule specificity and loading, while the final module contains a TE domain for product off-loading. (For example, in the erythromycin PKS 6-deoxyerythronolide B synthase (DEBS), the DEBS1 polypeptide includes AT, ACP, KS, AT, KR, ACP, KS, AT, KR, and ACP domains, the DEBS2 polypeptide includes KS, AT, ACP, KS, AT, DH, ER, KR, and ACP domains, and the DEBS3 polypeptide includes KS, AT, KR, ACP, KS, AT, KR, ACP, and TE domains.) While FAS TE domains essentially catalyze hydrolysis, releasing a linear free acid, certain PKS TE domains cleave their reactive polyketide substrate's thioester linkage by catalyzing an intramolecular polyketide cyclization step.

Much effort has gone into both the characterization and engineering of FAS and Type I PKS domain structure. For example, catalytic domains derived from different PKSs have been joined in new combinations; see, e.g., Menzella et al. (2005) “Combinatorial polyketide biosynthesis by de novo design and rearrangement of modular polyketide synthase genes” Nat Biotechnol 23:1171-1176, Sherman (2005) “The Lego-ization of polyketide biosynthesis” Nat Biotechnol 23(9):1083-1084, and Jenke-Kodama and Dittmann (2005) “Combinatorial polyketide biosynthesis at higher stage” Mol Syst Biol 1:E1-E2 (doi:10.1038/msb4100033). See also, Kodumal et al. (2004) “Total synthesis of long DNA sequences: Synthesis of a contiguous 32-kb polyketide synthase gene cluster” Proc Nat Acad Sci 44:15573-15578. Some commercial efforts involve bioengineering of various type I PKS enzymes, for example, by Kosan Biosciences (www (dot) kosan (dot) corn) and Biotica Technology Limited (www (dot) biotica (dot) co (dot) uk). A variety of type I FAS and PKS proteins, both naturally occurring and recombinant, are thus well known in the art (and additional examples can be identified on the basis of homology, three-dimensional structure, and/or enzymatic activity or created as described herein) and can be adapted to the practice of the present invention.

### Type III Polyketide Synthases

In contrast to type I PKSs, the type III PKS enzyme family, currently known to include at least fifteen functionally divergent beta-ketosynthases of plant and bacterial origin, is characterized by homology to chalcone synthase (CHS), the ubiquitous first-discovered plant PKS whose chalcone product forms the scaffold of numerous important flavonoid, isoflavonoid, and anthocyanin natural products.

Like the non-iterative ketoacyl-synthase III (KAS III) condensing enzymes of fatty acid biosynthesis (FAS) from which they apparently evolved, the iterative type III PKSs are structurally simple homodimers of the αβαβα-fold core domain conserved among all beta-ketosynthases and thiolases. Also like their KAS III progenitors, each approximately 400 amino acid type III PKS monomer utilizes a Cys-His-Asn catalytic triad within an internal active site cavity to condense an acetyl unit, typically derived from the decarboxylation of a malonyl moiety, to a starter molecule covalently attached to the catalytic cysteine through a thioester linkage. CoA-linked starter molecules and malonyl units are presented to the catalytic triad by way of a narrow CoA-binding tunnel, which connects the buried type III PKS active site cavity to the outside solvent. Quite unusually, as KAS III and other FAS and PKS condensing enzymes require malonyl-ACP, type III PKSs typically utilize CoA-linked malonyl as the source of acetyl units for polyketide extension. In another departure from their KAS III progenitors, type III PKSs are generally both iterative and multi-functional, typically catalyzing three polyketide extensions of their preferred starter molecules prior to catalyzing six-membered ring formation via an intramolecular cyclization of the resulting polyketide intermediate in the same active site cavity.

Despite their continued structural simplicity, type III PKS enzymes have evolved to catalyze an impressive repertoire of functionally divergent and mechanistically complex activities. These enzymes vary in their choice of starter molecule (ranging in size, e.g., from acetyl- to caffeoyl-CoA), in the number of polyketide extension steps they normally catalyze (e.g., between one and four), and also in their cyclization specificity and mechanism of intramolecular ring formation (e.g., C6->C1 Claisen, C2->C7 aldol, or lactone formation either from C5 carbonyl oxygen->C1 carbon of the thioester or from hydrolyzed C1 carboxylate oxygen->C5).

High-resolution x-ray crystal structures of plant CHS-like enzymes have facilitated the identification of both the structural and mechanistic bases for conserved as well as functionally divergent elements of type III PKS substrate specificity and catalysis. The first of these structures, that of alfalfa CHS2 (Ferrer et al. (1999) “Structure of chalcone synthase and the molecular basis of plant polyketide biosynthesis” Nat. Struct. Biol. 6:775-784), revealed the type III PKS overall fold and dimerization interface, important CoA-binding residues, and the CoA-binding tunnel, as well as the internal active site cavity containing the Cys-His-Asn catalytic triad. The three-dimensional elucidation of CHS's active site architecture, accompanied by site-directed mutagenesis of catalytic residues, allowed a much deeper mechanistic exploration of type III PKS catalysis than was possible before, although earlier biochemical studies had succeeded in identifying the catalytic cysteine and the reaction sequence by which CHS catalyzes chalcone formation from three malonyl-CoA extender molecules and a p-coumaroyl-CoA starter molecule derived from phenylalanine.

Subsequent homology modeling of other plant CHS-like enzymes implied that steric modulation of the size and shape of the type III PKS active site cavity was responsible for much of the functional divergence observed in various members of this family. This ‘steric modulation’ hypothesis was supported by the crystal structure of a 2-pyrone synthase (2PS) from Gerbera hybrida (daisy), which uses a much smaller active site cavity to catalyze only two acetyl extensions of an acetyl-CoA starter prior to lactone cyclization (Jez et al. (2000) “Structural control of polyketide formation in plant-specific polyketide synthases” Chem. Biol. 7:919-930). Interestingly, only three structure-guided active site mutations were required to fully convert alfalfa CHS2 into a functional 2-PS (Jez et al., supra).

Additional crystal structures have illuminated the structural basis of functional diversity in two classes of type III PKS enzymes whose mechanistic divergence could not easily be explained using homology modeling. The crystal structure of a pine stilbene synthase (STS) and subsequent mutagenic conversion of the alfalfa CHS model system to a functional STS resulted in the identification of the thioesterase-like “aldol switch” hydrogen-bonding network responsible for the puzzling C2-C7 aldol cyclization specificity of stilbene synthases, which had previously eluded explanation, despite the use of homology models and site-directed mutagenesis (Austin et al. (2004) “An aldol switch discovered in stilbene synthases mediates cyclization specificity of type HE polyketides synthases” Chem Biol 11(9):1179-94). Although STS specificity has evolved from CHS enzymes on more than one occasion, additional crystal structures of STS enzymes from peanut and grape (see, e.g., Shomura et al. (2005) “Crystal structure of stilbene synthase from Arachis hypogaea” Proteins 60(4):803-6) confirm the structural and mechanistic conservation of the aldol switch, despite the lack of a consensus STS sequence.

While the aforementioned structurally characterized plant enzymes share around 75% amino acid sequence identity with each other and with CHS (in general, functionally divergent plant type III PKSs typically share around 50-90% identity with each other), bacterial type III PKS enzymes are more divergent, typically sharing 25-35% amino acid sequence identity with plant and other bacterial type III PKS enzymes. Sequence alignments confirm the conservation in bacterial type III PKSs of both the Cys-His-Asn catalytic triad and a few other apparently structurally-important motifs, but these alignments also predict significant bacterial divergence from plant enzymes in the identity and reactivity of other residues lining their active site cavities.

The crystal structure of a 1,3,6,8-tetrahydroxynaphthalene (THN) synthase (THNS) enzyme from Streptomyces coelicolor was solved to illuminate the structural basis for this type III PKS enzyme's unusual catalytic ability (Austin et al. (2004) “Crystal structure of a bacterial type III polyketide synthase and enzymatic control of reactive polyketide intermediates” J Biol Chem 279(43):45162-74). This enzyme catalyzes four acetyl extensions of a malonyl-CoA starter molecule, accompanied by both Claisen and aldol condensation-mediated cyclizations to form a fused two-ring scaffold. The structure confirmed the preservation of the overall type III PKS fold, as well as the homology-predicted presence of additional active site cysteines. One of these additional cysteines is necessary for the THNS reaction, and has been proposed to act as a biochemical protecting group for the reactive polyketide intermediate, thus preventing derailment of polyketide extension through premature intramolecular cyclization. The THNS crystal structure also revealed an unexpected tunnel in the floor of the THNS active site cavity, likely responsible for the unusual ability of THNS enzymes to catalyze five polyketide extension steps using a long fatty acyl-CoA starter. This novel tunnel, occupied in the crystal structure by a polyethylene glycol (PEG) molecule, likely binds the long aliphatic tail of fatty acyl non-physiological starter molecules during progressive polyketide extension steps, thus maintaining a relatively linear orientation of the growing chain that provides THNS an alternative mechanism to prevent termination of polyketide extension via intramolecular cyclization (Austin et al. (2004) “Crystal structure of a bacterial type III polyketide synthase and enzymatic control of reactive polyketide intermediates” J Biol Chem 279(43):45162-74). More recently, a second bacterial type III PKS crystal structure by another group also revealed a similar THNS-like novel tunnel (Sankaranarayanan et al. (2004) “A novel tunnel in mycobacterial type III polyketide synthase reveals the structural basis for generating diverse metabolites” Nat Struct Mol Biol 11(9):894-900). In addition to the novel slime mold enzymes discussed herein, other novel functionally divergent plant type III PKS enzymes that catalyze more polyketide extension steps than THNS (the previous type III record holder) have also been recently discovered and characterized; see, e.g., Abe et al. (2004) “The first plant type III polyketide synthase that catalyzes formation of aromatic heptaketide” FEBS Lett 562(1-3):171-176 and Abe et al. (2005) “A plant type III polyketide synthase that produces pentaketide chromone” J Am Chem Soc 127(5):1362-3.

Additional details and description of the type III PKS enzyme superfamily are reviewed in Austin and Noel (2003) “The chalcone synthase superfamily of type III polyketide synthases” Nat Prod Rep 20:79-110. A variety of type III PKSs, both naturally occurring and recombinant, are thus well known in the art (and additional examples can be identified on the basis of homology, three-dimensional structure, and/or enzymatic activity or created as described herein) and can be adapted to the practice of the present invention.

### Recombinant Fusion Proteins

One aspect of the present invention involves a novel gene and/or protein structure that covalently links the biosynthetic capabilities of two very different types of polyketide/fatty acid synthase enzymes, for example, type I PKSs/FASs and type III PKSs. This covalent linkage represents a significant technological innovation that can be used, e.g., to expand the biosynthetic repertoire of various PKS systems as well as to produce novel fatty acid derived products.

As described in greater detail below in Example 1, two naturally-occurring prototypical fusion proteins of this invention were discovered using bioinformatic analyses of publicly-available genomic sequencing data from the slime mold Dictyostelium discoideum. These two predicted multi-domain polypeptides, respectively named “Steely1 ” and “Steely2”, are each roughly 3000 amino acids in length and are located on different chromosomes. The first roughly 2600 residues of each putative steely protein shares homology with the first six of seven catalytic domains that make up type I FAS enzymes, as well as individual modules of type I PKS enzymes (which have clearly evolved from a type I FAS ancestor). The last of these six Steely N-terminal domains contains a phosphopantethiene (Ppant) attachment site.

In FAS and type I PKS enzymes, intermediates are attached by a thioester bond to the prosthetic Ppant arm, which transfers intermediates between FAS/PKS domain active sites during polyketide extension and reduction, and also to the active site of a C-terminal (seventh) thioesterase (TE) domain for final product off-loading. In contrast, the final roughly 400 amino acids of the steely proteins are homologous with type III PKS enzymes. This substitution of type III PKS domains for C-terminal TE domains, in the context of the otherwise conserved FAS-like domain arrangement of the Steely proteins, suggests direct transfer of the prosthetic Ppant-bound polyketide or fatty acid products of the six N-terminal domains to this seventh iterative PKS domain.

Each of these C-terminal type III PKS domains has been cloned and heterologously expressed in E. coli, and their in vitro catalytic activities confirm that they are each functional iterative PKS domains with distinct substrate preferences. The crystal structure of the Steely1 C-terminal domain has also been solved, confirming these domains' conservation of the typical type III PKS internal active site, Cys-His-Asn catalytic triad, and homodimeric domain assembly. These initial experimental results indicate that these Steely C-terminal type III PKS domains can carry out additional and iterative polyketide extension of the intermediate product(s) of the N-terminal FAS-like domains, rather than merely functioning as simple TE-like hydrolytic domains.

This conclusion has profound technological implications for bioengineering of both type I and type III PKS systems. Together, these observations suggest that the evolutionarily refined Steely sequences represent untapped templates for the covalent and functional fusion of type I and type III systems. For example, exploitation of the Steely fusion protein linker sequences and/or type III PKS domains can facilitate the combinatorial coupling of any number of N-terminal modular or iterative type I FAS or PKS modules to a growing collection of functionally distinct iterative type III PKS enzymes (including, e.g., the Steely 1 and 2 type III PKS domains).

In this regard, the similar overall architectures of modular type I PKSs and animal type I FASs, as revealed by recent crystal structures, are informative. Two similar structures of the same two-domain fragment (KS-AT) from two different PKS modules resemble the arrangement of the first two N-terminal domains in the larger multidomain architecture of animal FAS, which in turn resembles the first six domains (i.e. all but the final CHS-like domain) of the Steely 1 and 2 hybrids from Dictyostelium described herein. (See Tang et al. (2007) “Structural and mechanistic analysis of protein interactions in module 3 of the 6-deoxyerythronolide B synthase” Chem. Biol. 14(8):931-43, Tang et al. (2006) “The 2.7-Angstrom crystal structure of a 194-kDa homodimeric fragment of the 6-deoxyerythronolide B synthase” Proc Natl Acad Sci USA 103(30):11124-9, and Maier et al. (2006) “Architecture of mammalian fatty acid synthase at 4.5 A resolution” Science 311(5765):1258-62, as well as Example 1 hereinbelow.) These architectural similarities reinforce the relevance of the natural Steely hybrids to informing the engineering of type III PKS hybrid systems using either type I FAS or type I PKS N-terminal domains.

Construction of type I PKS/FAS-type III PKS fusion proteins, including, for example, libraries of such fusion proteins, can increase the efficiency of PKS- or FAS-derived acyl substrate delivery to the covalently tethered type III enzymes by allowing direct transfer of the type I domain's product to the type III active site without the traditional need for TE-catalyzed hydrolytic release as a free acid followed by the subsequent CoA ligase-catalyzed reactivation of the free acid as a CoA thioester. Likewise, the typically iterative polyketide extension and subsequent aromatic cyclization of acyl-primed substrates by relatively small type III PKS enzymes represents a substantial addition to the toolbox of type I PKS bioengineers; utilization of the Steely template and construction of PKS/FAS type I-PKS type III fusion proteins can significantly expand the size and diversity of type I PKS products, while adding less than 400 amino acids to the recombinant, size-limited multi-enzyme biosynthetic proteins.

Bioengineered control and optimization of modular PKS biosynthesis is currently at least partially limited by the enormous size of modular PKS genes and multi-enzymatic domain proteins. Addition or substitution of various type TR PKS domains into various iterative and modular FAS and PKS multi-domain proteins, as suggested by the evolutionarily optimized Steely fusion proteins described herein, has the potential to greatly increase the scope of biosynthetic diversity available to type I PKS engineering, with minimal addition to the overall size of biosynthetic genes and resulting proteins. For example, substitution of approximately 400 residue iterative and multi-functional type III PKS domains in place of C-terminal TE domains in existing two-module combinatorial libraries of type I PKS bioengineered constructs (e.g., Menzella et al. (2005) “Combinatorial polyketide biosynthesis by de novo design and rearrangement of modular polyketide synthase genes” Nat Biotechnol 23:1171-1176) can convert the current triketide lactone products of these TE-terminated constructs into hydroxylated phloroglucinol, resorcinol, or naphthalene rings derived from hexaketide (or longer) linear intermediates.

Conversely, Steely-like efficient direct (“channeled”) delivery of needed type I FAS or PKS products as acyl substrates directly to a type III PKS active site (e.g., for further extension and intramolecular cyclization) can be ideal for optimizing transgenic introduction of desired type III catalytic activities into species that lack needed starter molecule substrates (or CoA ligases capable of activating them for type III PKS catalysis), where depletion of existing substrate pools is undesirable, or where introduction of the acyl substrates in diffusible form is undesirable. One such exemplary commercial bioengineered application involves transgenic transfer of type I PKS/FAS-type III PKS fusion genes into heterologous hosts for the purpose of conferring in vivo cooperative type I/III production of the hexanoyl-primed resorcinolic acid polyketide precursor of THC and related bioactive cannabis natural products (pharmaceutical targets). In combination with optional co-transformation of downstream prenylation enzymes or other methods, this strategy allows or improves heterologous in vivo production of cannabinoid natural products for various pharmaceutical or signal transduction purposes.

Recombinant Type I FAS/PKS-Type III PKS Fusion Proteins

Accordingly, one general class of embodiments provides a recombinant fusion protein that comprises at least one type I polyketide synthase domain or type I fatty acid synthase domain and a type LEI polyketide synthase domain.

The at least one type I polyketide or fatty acid synthase domain typically comprises one or more of: a ketoacyl synthase domain, an acyl transferase domain, a dehydratase domain, an enoyl reductase domain, a ketoreductase domain, and an acyl carrier domain (ACP, including a phosphopantetheine attachment site). The fusion protein optionally includes two or more, three or more, four or more, five or more, or even six or more such domains. For example, in one class of embodiments, the recombinant fusion protein includes type I fatty acid synthase ketoacyl synthase, acyl transferase, dehydratase, enoyl reductase, ketoreductase, and acyl carrier domains. The type III PKS domain optionally replaces a thioesterase (TE) domain in a type I FAS or type I PKS.

The domains can be arranged in essentially any order consistent with the desired activity of the fusion protein. However, by analogy with the domain organization of a variety of naturally occurring type I FASs and PKSs in which the TE domain is C-terminal to the other domains, in one exemplary class of embodiments the type III polyketide synthase domain is C-terminal to the at least one type I polyketide or fatty acid synthase domain.

The type I PKS or FAS domain and the type III PKS domain are optionally joined by a linker (e.g., when they are not separated from each other by other enzymatic domains in the fusion protein). The linker is optionally identical to, or derived from, a type I PKS or FAS (e.g., the same type I PKS or FAS as the type I domain, and including sequence adjacent to the type I domain), Steely1 (SEQ ID NO:1, e.g., residues 2629-2775 that link the AC domain and the type III domain of Steely1), or Steely2 (SEQ ID NO:2, e.g., residues 2473-2615 that link the AC domain and the type III domain of Steely2), or an amino acid sequence at least about 25% identical thereto (e.g., at least about 50%, at least about 75%, at least about 90%, at least about 95%, at least about 98%, or at least about 99% identical thereto.

As noted above, a wide variety of type I FAS and PKS proteins are known in the art, in which ketoacyl synthase, acyl transferase, dehydratase, enoyl reductase, ketoreductase, and acyl carrier domains are found in various orders and combinations. An extensive variety of such domains is thus available and can be adapted to the practice of the present invention. The recombinant fusion protein optionally also includes additional domains, e.g., additional domains found in type I PKS proteins such as a methyltransferase (MT) domain (e.g., the putative MT domain found in the Steely1 N-terminal portion between the AT and DH domains), which can be specific for either C- or O-methylation, or a KAS III or similar domain, preferably at the N-terminus of the fusion protein, to initiate (and modulate starter specificity of) type I PKS catalysis.

Similarly, a wide variety of type III PKSs are known in the art. Furthermore, type III PKSs typically have (or can be mutated to have) promiscuous starter substrate specificity, and changing the nature of the starter (in vivo or in vitro) usually affects subsequent steps (e.g., number of polyketide extensions catalyzed and/or mode of intramolecular product cyclization); the utility of type III PKSs in fusion proteins is thus not restricted to their physiological reactions. Moreover, as briefly described herein, available detailed knowledge of type III PKS structure/function relationships means that site-directed point mutants of essentially any type III PKS that result in alteration of substrate and product specificity can readily be made.

Examples of known functionally divergent wild-type type III PKSs from which type III PKS domains can be derived for inclusion in fusion proteins of the invention include, but are not limited to, chalcone synthase (CHS), stilbene synthase (STS), stilbenecarboxylate synthase (STCS), bibenzyl synthase (BBS), homoeriodictyol/eriodictyol synthase (BEDS), acridone synthase (ACS), benzophenone synthase (BPS), phlorisovalerophenone synthase (VPS), coumaroyl triacetic acid synthase (CTAS), benzalacetone synthase (BAS), 1,3,6,8-tetrahydroxynaphthalene synthase (THNS), phloroglucinol synthase (PhlD), dihydroxyphenylacetate synthase (DpgA), alkylresorcinol synthase (ArsB), alkylpyrone synthase (ArsC), aloesone synthase (ALS), pentaketide chromone synthase (PCS), octaketide synthase (OKS), the Steely2 C-terminal domain (differentiation acyl phloroglucinol synthase or DAPS), and benzalacetone synthase. Various of these known wild-type enzymes (or mutated versions of them) are capable, for example, of incorporating a wide range of thioester-linked acyl or similar starter substrates, then catalyzing between one and seven polyketide extension steps using malonyl- or methylmalonyl-thioester extender molecules, and finally producing either linear decarboxylated methylketones or an intramolecularly cyclized product where some combination of Claisen, aldol, or lactone cyclization mechanisms ultimately produce polyhydroxylated single- or multiple-ringed phloroglucinol, acyl phloroglucinol, chalcone acridone, bibenzyl, acyl resorcinol, acyl resorcinolic acid, stilbene, stilbene acid, tetrahydroxynaphthalene, acyl chromone, acyl lactone, or acyl pyrone products, for example. One type III PKS was recently also shown to synthesize “SEK4” aromatic octaketide cyclized products (previously thought to be made only by type II PKSs); see Abe et al. (2005) “Engineered biosynthesis of plant polyketides: chain length control in an octaketide-producing plant type III polyketide synthase” J Am Chem. Soc. 127(36):12709-16.

In addition to these examples, many other experimentally characterized type III PKS domains are also known, that like the Steely1 C-terminal domain display a fairly distinct (but not necessarily unique) set of in vitro substrate and product specificities, regardless of whether their in vivo function is yet known. Isoenzymes from multiple species are also available, and can offer slightly different substrate preferences or kinetic parameters. Moreover, the number of type III PKS protein sequences publicly available in databases is constantly increasing. See, for example, the protein and nucleotide databases available at the National Center for Biotechnology Information through the Entrez browser at www (dot) ncbi (dot) nlm (dot) nih (dot) gov/entrez/query (dot) fcgi, in which a wide variety of protein and nucleotide sequences for type III PKS proteins (and, indeed, the other types of proteins and domains optionally utilized in the methods and compositions of the present invention) are described.

An extensive array of recombinant type I-type III fusion proteins is readily constructed. For example, in terms of generating further engineered diversity from a type I PKS system, combinatorial selection of essentially any type III PKS domain fused, e.g., to the C-terminus, of essentially any natural or artificial type I PKS mono-, di- or tri-modular construct can diversify the resulting products. Examples of such type I constructs include the previously engineered DEBS di-domain constructs of Menzella et al. (2005) supra. An artificial construct joining the first two DEBS modules to the TE domain (normally on module 6) produced triketide lactones. Subsequent mixing/matching of DEBS modules/domains in similar constructs diversified the triketide lactone output. Simply substituting one (or various different) type III PKSs (including, but not limited to, DAPS, CHS, STS, THNS, OKS, etc.) for the TE domains in these constructs, with appropriate linkers between the ACP and the C-terminal type III PKS domain, allows much more significant diversification (e.g., varied numbers of additional non-reductive polyketide extension steps, as well as additional cyclization/off-loading options other than simple (TE-like) hydrolysis-mediated formation of lactones). The linkers between the acyl carrier domain and the C-terminal type III PKS domain are optionally derived from the linkers of the Steely1 and Steely2 proteins described herein, for example.

Another exemplary recombinant fusion protein includes the non-iterative type III PKS benzalacetone synthase fused to a type I FAS. The fusion protein is optionally used to produce an aliphatic methylketone product.

Another exemplary recombinant fusion protein includes the hexanoyl-specific Steely2 N-terminal domains fused to a suitable (existing or engineered) type III PKS that catalyzes aldol cyclization following three rounds of polyketide extension of hexanoyl. This fusion protein would form olivetol or olivitolic acid, depending upon whether STS-like decarboxylative aldol cyclization or STCS-like carboxyl-retaining aldol cyclization occurs. Olivetolic acid is an on-pathway intermediate (and the polyketide core) of psychoactive Cannabis natural products such as THC. Thus an olivetolic acid- or olivetol-producing steely fusion protein can serve as a useful substrate-channeling heterologous engineering tool for the first steps of cannabinoid natural product biosynthesis. While type III PKSs isolated from Cannabis have thus far not catalyzed the desired activity in vitro, the appropriate activity can be engineered either from STS, STCS, or ArsB (which catalyze the desired number of extensions and cyclization but utilize different starter substrates) or alternatively from either the Steely1 or Steely2 C-terminal domain (which already prefer a hexanoyl starter but catalyze different cyclizations).

Yet another exemplary recombinant fusion protein includes either the Steely2 N-terminal domains or a typical type I FAS (exclusive of the TE domain) fused to ArsB or one of several similar alkylresorcinol-forming type III PKSs from rice or sorghum. This fusion protein is useful for the channeled heterologous biosynthesis of alkylresorcinols of varying lengths. Alkylresorcinols are necessary for protective cyst formation in Azotobacter, and also serve as pathway intermediates leading to sorgoleone and related allelopathic natural products in crop plants such as rice and sorghum. Moreover, the above and similar alkyl resorcinols (including those resulting from STCS-like carboxyl-retaining aldol cyclization) can also serve as pathway intermediates leading to anacardic acid and other urushiols. These are the active (anti-pest) skin irritants in poison ivy and related plants (including lacquer and related plant products) and thus could potentially be useful for bioengineered plant defense. Given their potent effect upon animal cells, bioengineered urushiol derivatives can also prove useful under other biological or medicinal circumstances.

Yet another exemplary recombinant fusion protein includes a fusion of a medium- or long-chain (unbranched and saturated) fatty acid-producing N-terminal region (like Steely2 or type I FAS, respectively) to a C-terminal BAS-like type III PKS, allowing the facile channeled production of straight-chain methylketones of different lengths. Methylketones are components of the essential oils of many plants, and are quite effectively used by plants to repel insect pests. Nature produces fatty acid-derived methylketones via a TE-like (alpha-beta-hydrolase-fold) enzyme called methylketone synthase (MKS), which hydrolyzes and decarboxylates a beta-ketoacyl fatty acyl thioester of unknown origin. However, BAS is a type III PKS that performs a similar hydrolytic decarboxylation of a diketide intermediate that it forms by one round of polyketide extension of a phenylpropanoid (phenylalanine-derived) starter moiety (to form an intermediate leading to the aroma of raspberries). The residues contributing to BAS's unusual reaction specificity (non-iterative extension leading to hydrolysis and decarboxylation) are known, and so a type III PKS catalyzing the formation of fatty acid-primed methylketones can be engineered by altering the starter specificity of BAS, or alternatively by engineering BAS non-iterativeness and hydrolytic decarboxylative activity into some other type III PKS that accommodates a fatty acid starter. Notably, several type III PKSs (including CHS, another phenylpropanoid-utilizing enzyme) are able to quite efficiently utilize long-chain fatty acid starters, presumably by accessing the acyl-binding tunnel first observed in the THNS crystal structure.

Yet another exemplary recombinant fusion protein includes a C-terminal VPS (or similar) domain with N-terminal type I PKS domains producing short branched intermediates. This fusion facilitates the channeled biosynthesis of branched acyl phloroglucinols such as phlorisovalerophenone. This and similar products are on-pathway intermediates leading to the bitter acids (such as humulone and lupulone) found in hops. These compounds are vital flavor components of beer, and possess other useful medicinal and neutraceutical properties as well.

It will be evident that this list of examples is far from exhaustive, as the possible biosynthetically-productive combinations of existing or engineerable type I and type III domains is quite extensive.

The recombinant fusion protein optionally includes one or more domains derived from the Steely1 or Steely2 proteins described herein (SEQ ID NO:1 and 2, respectively), including conservative variants thereof as well as variants with altered function (e.g., altered starter, extender, and/or product specificities). For example, the fusion protein optionally includes one or more of a ketoacyl synthase domain, acyl transferase domain, dehydratase domain, enoyl reductase domain, ketoreductase domain, and acyl carrier domain derived from Steely1 or Steely2. In one class of embodiments, the fusion protein includes the Steely1 PKS III domain (approximately residues 2776-3147 of SEQ ID NO:1, e.g., within about 20, about 10, or about 5 residues of, or at, the indicated position(s)); the Steely1 PKS III domain and the linker N-terminal to it (approximately residues 2629-3147 of SEQ ID NO:1); the Steely1 AC domain, PKS III domain, and the linker connecting them (approximately residues 2560-3147 of SEQ ID NO:1); or the Steely1 linker connecting the AC and PKS III domains (approximately residues 2629-2775 of SEQ ID NO:1); or an amino acid sequence at least about 25% identical thereto (e.g., at least about 50%, at least about 75%, at least about 90%, at least about 95%, at least about 98%, or at least about 99% identical thereto). In another class of embodiments, the fusion protein includes the Steely2 PKS III domain (approximately residues 2616-2968 of SEQ ID NO:2); the Steely2 PKS III domain and the linker N-terminal to it (approximately residues 2473-2968 of SEQ ID NO:2); the Steely2 AC domain, PKS III domain, and the linker connecting them (approximately residues 2412-2968 of SEQ ID NO:2); or the Steely2 linker connecting the AC and PKS III domains (approximately residues 2473-2615 of SEQ ID NO:2); or an amino acid sequence at least about 25% identical thereto (e.g., at least about 50%, at least about 75%, at least about 90%, at least about 95%, at least about 98%, or at least about 99% identical thereto).

Optionally, the fusion protein includes 50 or more contiguous amino acids of SEQ ID NO:1 or SEQ ID NO:2 (e.g., 100 or more, 200 or more, 300 or more, 400 or more, 500 or more, 1000 or more, 1500 or more, 2000 or more, or even 2500 or more), or an amino acid sequence at least about 25% identical thereto (e.g., at least about 50%, at least about 75%, at least about 90%, at least about 95%, at least about 97%, or at least about 99% identical thereto).

In the recombinant type I PKS/FAS-type III PKS fusion protein, typically the at least one type I polyketide synthase domain or type I fatty acid synthase domain catalyzes conversion of one or more first precursors to an intermediate. For example, the type I domain(s) can collectively catalyze the conversion of a starter unit and one or more extender units into an acyl intermediate. The intermediate is covalently bound to the fusion protein. The fusion protein typically contains an AC domain with a phosphopantetheine attachment site, and the intermediate (e.g., the acyl intermediate) is covalently bound to the phosphopantetheine group as a thioester. Rather than being released (for example, by hydrolysis or cyclization via action of a type I PKS or FAS TE domain), the covalently bound intermediate is transferred to the type III domain. The type III polyketide synthase domain catalyzes conversion of the intermediate to a polyketide product, which is typically released from the enzyme (i.e., the product is diffusible).

Additional Recombinant Fusion Proteins

One aspect of the invention relates generally to recombinant fusion proteins in which domains that, in the context of their parental enzymes, do not ordinarily transfer an intermediate directly between them but that, in the context of the fusion protein, do engage in such transfer. For example, a domain derived from a parental enzyme that releases a diffusible product can instead, in the context of the recombinant fusion protein, produce a covalently bound moiety (the product of the domain) that serves as a substrate for the other domain in the fusion protein.

Thus, one general class of embodiments provides a recombinant fusion protein that comprises at least a first domain that catalyzes conversion of one or more precursors to an intermediate, which intermediate is covalently bound to the fusion protein, and a second domain that catalyzes conversion of the intermediate to a product. The product is typically released by the second domain and is free to diffuse away, rather than being covalently attached to the fusion protein. Domains in the fusion protein are optionally connected by polypeptide linker(s), as noted above.

The first and second domains used to create the recombinant fusion protein are derived from different parental polypeptides. Typically, the first and second polypeptide are enzymes of different types or belonging to different families. For example, when the first domain is a type I PKS domain, the second domain is other than a type I PKS domain. Similarly, when the first domain is a non-ribosomal peptide synthetase (NRPS) domain, the second domain is other than an NRPS domain. Optionally, when the at least one first domain comprises a type I PKS domain or an NRPS domain, the second domain is other than a type I PKS domain or an NRPS domain.

In one class of embodiments, the product is released by the second domain, and the second domain is other than a thioesterase domain. The second domain optionally replaces a thioesterase domain (or another product-releasing domain) in a first enzyme from which the first domain is derived. The second domain is optionally C-terminal to the first domain.

In one class of embodiments, the first domain is derived from an enzyme that catalyzes conversion of the one or more precursors to a diffusible product. For example, the first domain can be derived from a type I FAS, a type I PKS, a non-ribosomal peptide synthetase (NRPS), or a mixed NRPS/PKS. While the parental enzyme releases a diffusible product, in the context of the recombinant fusion protein, the domain derived from the enzyme produces a covalently bound moiety.

In one class of embodiments, the second domain is derived from an enzyme that catalyzes conversion of a diffusible substrate to the product (or to another product). For example, the second domain can be derived from a type II PKS, a type III PKS, or another enzyme having a thiolase fold and sharing the type III PKS catalytic triad of Cys-His-Asn. (Type III PKS family members are also members of the much larger evolutionarily-related thiolase-fold group of enzymes; several related thiolase-fold family members, including KAS III, very long chain fatty acid elongase enzymes from type II FAS systems, and the HMG-CoA synthetases from cholesterol biosynthesis, also share the type III PKS catalytic triad of Cys-His-Asn.) While the parental enzyme (and optionally the second domain in the context of the parental enzyme) acts on a diffusible substrate, in the context of the recombinant fusion protein, the domain derived from the enzyme acts on a covalently bound substrate (the intermediate that results from the action of the first domain). Exemplary diffusible substrates include, but are not limited to, thioester substrates covalently linked to CoA or soluble ACP (or a pantetheine analog or mimic such as sNAC).

Exemplary recombinant fusion proteins include the type I FAS or PKS-type III PKS fusions described above. Thus, one exemplary class of embodiments provides a recombinant fusion protein wherein the first domain is a type I polyketide synthase domain or type I fatty acid synthase domain and the second domain is a type III polyketide synthase domain, and wherein the fusion protein comprises an acyl carrier domain to which the intermediate is covalently bound. Typically, the product is released by the type III polyketide synthase domain. As for the embodiments above, in fusion proteins that include more than one first domain, the first domains can collectively catalyze conversion of the precursor(s) to the intermediate.

In one class of embodiments, the fusion protein includes a type I PKS or FAS domain as the first domain, an acyl carrier domain, and a beta-ketosynthase domain as the second domain. The type I domain is optionally N-terminal of the betaketosynthase domain. The covalent linkage of the first and second domains can, for example, facilitate direct transfer of any small molecule reaction intermediate from the covalently-linked AC domain (containing a phosphopantetheine attachment site) of any N-terminal multi-domain type I FAS- or type I PKS-like construct to the adjacent active site of any C-terminal single-domain beta-ketosynthase domain, where this latter C-terminal domain would under natural circumstances instead utilize thioester substrates linked to CoA or a soluble (stand-alone) ACP domain (or a similar related phosphopantetheine carrier).

In one class of embodiments, the second domain is an iterative or aromatic iterative PKS (e.g., an iterative type III PKS or type II PKS domain). In another class of embodiments, the second domain is a non-iterative PKS domain; for example, benzalacetone synthase can be fused to a type I FAS to produce a fusion protein producing an aliphatic methylketone product. In some embodiments, the second domain is a non-cyclizing PKS. In other embodiments, the second domain is a cyclizing PKS. For example, the second domain can catalyze an aldol or Claisen reaction (forming carbon-carbon bonds) or a lactonization reaction (forming a carbon-oxygen bond). Such activities can occur exclusively (e.g., Claisen in CHS and Steely2, aldol in STS) or together (e.g., Claisen and aldol in tetrahydronaphtalene synthase).

As noted, the second domain is optionally derived from a non-type III PKS enzyme from a family having a similar enzyme fold, homodimeric assembly, Cys-His-Asn catalytic triad in an internal active site cavity, and substrate delivery via a phosphopantetheine thioester as the type III PKS family. See, e.g., Austin and Noel (2003) Nat Prod Rep 20:79-110 for additional information on such related enzymes, as well as Keatinge-Clay et al. (2004) “An antibiotic factory caught in action” Nat Struct Mol. Biol. 11(9):888-93 for an exemplary type II PKS structure; Pojer et al. (2006) “Structural basis for the design of potent and species-specific inhibitors of 3-hydroxy-3-methylglutaryl CoA synthases” Proc Natl Acad Sci USA. 103(31):11491-6 for an exemplary HMGCS structure; Scarsdale et al. (2001) “Crystal structure of the Mycobacterium tuberculosis beta-ketoacyl-acyl carrier protein synthase III” J Biol. Chem. 276(23):20516-22 and Qiu et al. (1999) “Crystal structure of beta-ketoacyl-acyl carrier protein synthase III. A key condensing enzyme in bacterial fatty acid biosynthesis” J Biol. Chem. 274(51):36465-71 for structures of KAS III enzymes with specificity for long-chain (unusual) and short chain (typical) fatty acid substrates, respectively; and Blacklock and Jaworski (2006) “Substrate specificity of Arabidopsis 3-ketoacyl-CoA synthases” Biochem Biophys Res Commun. 346(2):583-90 for additional information on beta-ketoacyl-CoA synthases (KCS) homologous to type III PKSs.

Thus, exemplary second domains include domains derived from, e.g., non-iterative HMG-CoA synthase (HMGCS) or beta-ketoacyl-ACP synthase III (KAS enzymes. While typical KAS III enzymes select short straight- or branched-chain acyl starters, at least one KAS III from Mycobacterium (MtFabH) prefers long chain fatty acids as substrate. For example, a fusion protein of the invention can include a type I FAS or PKS domain fused to a C-terminal HMG-CoA synthase or KAS III domain.

Similarly, the second domain can be a beta-ketoacyl-CoA synthase domain. The beta-ketoacyl-CoA (KCS) synthases are a class of type III PKS-like enzymes involved in the biosynthesis of very long chain fatty acids (VLCFAs), in seed coats and other specialized tissues, via extension of more conventional fatty acid intermediates derived from typical fatty acid biosynthesis. Sequence alignments reveal Cys-His-Asn active site conservation with type III PKSs.

As another example, the second domain can be a type II PKS domain, e.g., a beta-ketosynthase (KS-alpha) domain. Like type III PKSs, type II PKSs are also typically small aromatic iterative enzymes that can utilize type I PKS-generated substrates. Type II PKSs are heterodimers consisting of a catalytically active beta-ketosynthase (KS-alpha) domain as well as a structurally required second homologous domain with no ketosynthase activity (KS-beta, also called CLF for Chain Length Factor). Both of these type II PKS domains are preferably encoded adjacently, e.g., joined by a linker and C-terminal to one or more type I PKS first domains. Without limitation to any particular mechanism, the fusion protein would thus typically form two independent type II PKS heterodimers at the C-terminus of each N-terminal type I PKS dimeric assembly. This quaternary arrangement is not significantly different then that formed by mammalian FAS proteins, which appear to utilize monomeric C-terminal TE domains (rather than the homodimeric TE domains of type I PKS systems).

Recombinant fusion proteins of the invention optionally include Non-Ribosomal Peptide Synthetase (NRPS) domains, e.g., as first domains or in combination with type I PKS first domains. Exemplary recombinant fusion proteins can thus include NRPS systems or mixed NRPS/type I PKS systems at their N-terminus, and optionally a type III PKS or similar domain at their C-terminus. Non-ribosomal peptide synthetases are covalently attached multi-domain assembly lines that form peptide linkages between (common or specialized) amino acids, in much the same specificity-programmed and stepwise modular fashion as polyketides are formed by type I PKSs. NRPS domains are often found integrated with type I PKS domains in mixed systems that produce natural products containing both polyketide and amino acid moieties. NRPS also utilize covalent attachment of intermediates on ACP-like carrier proteins or domains, called CPs or PCPs (peptidyl carrier proteins) to reflect their peptidyl cargo. Aryl carrier proteins or domains are similarly utilized by certain NRPSs. Other typical NRPS domains include adenylation (A) and condensation (C) domains, to activate specific amino acid substrates via formation of a thioester linkage to CP, and to catalyze amide bond formation with the growing peptidyl chain. The naturally-occurring mixed systems and common use of carrier proteins suggests that a strategy involving direct loading from a type I system's AC domain to an adjacent type III PKS or similar domain is applicable to mixed modular systems, e.g., where the type I PKS portion is C-terminal to the NRPS domains (and thus interacts with the type III system). A similar strategy can also apply with no or minimal further engineering to direct loading between a NRPS CP domain and an adjacent type III PKS domain (whether in a fusion protein including an alternatively-ordered mixed type I PKS/NRPS arrangement or one including purely NRPS N-terminal domains).

For additional description of NRPS and mixed NRPS/PKS systems, see, e.g., Hill (2005) “The biosynthesis, molecular genetics and enzymology of the polyketide-derived metabolites” Nat Prod Rep. 23(2):256-320, Challis and Naismith (2004) “Structural aspects of non-ribosomal peptide biosynthesis” Curr Opin Struct Biol. 14(6):748-56, Finking and Marahiel (2004) “Biosynthesis of nonribosomal peptides” Annu Rev Microbiol. 58:453-88, Schwarzer et al. (2003) “Nonribosomal peptides: from genes to products” Nat Prod Rep. 20(3):275-87, Lautru and Challis (2004) “Substrate recognition by nonribosomal peptide synthetase multi-enzymes” Microbiology 150:1629-1636 and Huang et al. (2001) “A multifunctional polyketide-peptide synthetase essential for albicidin biosynthesis in Xanthomonas albilineans” Microbiology 147:631-642. See also, Hillson and Walsh (2003) “Dimeric structure of the six-domain VibF subunit of vibriobactin synthetase: mutant domain activity regain and ultracentrifugation studies” Biochemistry 42(3):766-75, which demonstrates that at least some NRPS polyproteins associate as dimeric assemblies like type I FAS and PKS systems. As with combinatorial engineering of type I PKS modules discussed above, much effort has been directed toward isolated NRPS model systems (e.g., di-modular systems), including mixing and matching domains and switching out different C-terminal TE domains to change product specificity. Exemplary di-modular NRPS model systems and modular engineering studies including TE domain engineering are described in, e.g., Duerfahrt et al. (2004) “Rational design of a bimodular model system for the investigation of heterocyclization in nonribosomal peptide biosynthesis” Chem. Biol. 11(2):261-71 and Schwarzer et al. (2001) “Exploring the impact of different thioesterase domains for the design of hybrid peptide synthetases” Chem. Biol. 8(10):997-1010; these and similar constructs can be adapted to the practice of the present invention.

In an exemplary fusion protein in which the first domain is an NRPS domain and the second domain is a type III PKS domain, direct transfer between the C-terminal CP domain of a one- or two-module NRPS system (such as those described above, for example) and the adjacent (e.g., C-terminal to the CP domain) covalently linked type III PKS domain can allow type III PKS-catalyzed polyketide extension of CP-thioester-activated amino acyl or dipeptide moieties, respectively. Phenylpropanoid-utilizing type III enzymes such as CHS, STS, BAS, etc. may optionally prime with NRPS A-domain activated phenylalanine, tyrosine, or histidine. Retention of the starter moiety's amine (normally lost during phenylpropanoid starter biosynthesis) can facilitate other interesting chemistries following type III PKS-catalyzed polyketide extension.

A related exemplary fusion protein includes one or more type I PKS domains (one of which is the first domain), one or more NRPS domains, and a type III PKS domain (as the second domain). This type of fusion protein can incorporate an NRPS-derived amino acyl starter into a type I PKS-extended product, which is then transferred like any other type I FAS/PKS ACP-bound thioester to the C-terminal type III PKS. In this way, some peptidyl or amino acyl characteristics can be incorporated into a type III PKS-extended product, with no direct interaction required between the NRPS and type III PKS machinery.

In one class of embodiments, the first domain is a type I polyketide synthase domain or type I fatty acid synthase domain, and the fusion protein comprises an acyl carrier domain to which the intermediate is covalently bound. In another class of embodiments, the first domain is an NRPS domain, and the fusion protein comprises a peptidyl carrier domain to which the intermediate is covalently bound. In one class of embodiments, the fusion protein comprises an acyl carrier domain (or a peptidyl carrier domain) to which the intermediate is covalently bound, and the second domain is selected from the group consisting of a beta-ketosynthase domain, an aromatic iterative polyketide synthase domain, a type III polyketide synthase domain, a type II polyketide synthase domain, a non-iterative polyketide synthase domain, an HMG-CoA synthetase domain, a ketoacyl-synthase III domain, and a beta-ketoacyl CoA synthase domain.

### Making Polyketides and Other Products

The fusion proteins of the invention can be used to produce products, for example, polyketide (or other) products that are novel, that are not naturally produced in a given cell type, in quantities greater than naturally produced in a given cell type, or the like. Accordingly, one aspect of the invention provides methods of making a product. In the methods, a recombinant fusion protein is provided. The fusion protein comprises a first domain that catalyzes conversion of one or more precursors to an intermediate, which intermediate is covalently bound to the fusion protein, and a second domain that catalyzes conversion of the intermediate to a product. One or more first precursors are contacted with the recombinant fusion protein, whereby the first domain catalyzes conversion of the precursor(s) to the intermediate and the second domain catalyzes conversion of the intermediate to the product. The recombinant fusion protein, first domain, second domain, etc. can be any of those described herein. Similarly, the precursor(s) can be any of those described herein and/or known in the art, for example, various acyl thioesters for fusion proteins including FAS or PKS domains, or natural or unnatural D- or L-amino acids for fusion proteins including NRPS domains.

For example, recombinant type I FAS or PKS-type III PKS fusion proteins can be used to produce polyketides. One class of embodiments thus provides methods of making a polyketide product. In the methods, a recombinant fusion protein comprising at least one type I polyketide synthase or type I fatty acid synthase domain and a type III polyketide synthase domain is provided. One or more first precursors are contacted with the recombinant fusion protein, whereby the at least one type I polyketide synthase or fatty acid synthase domain catalyzes conversion of the one or more first precursors to an intermediate, and the type III polyketide synthase domain catalyzes conversion of the intermediate (and optionally one or more second precursors) to the polyketide product. Typically, the intermediate is covalently bound to the fusion protein. For example, the type I PKS or FAS domain can catalyze conversion of one or more extender units and a starter unit (the first precursors) to an acyl intermediate which is covalently bound as a thioester to the prosthetic Ppant arm of an acyl carrier domain in the fusion protein; the type III PKS domain can then catalyze conversion of the intermediate, and typically additional extender unit(s) (the second precursors, which can be the same as or different from the first extender units), to the polyketide product. The product is typically diffusible.

In one class of embodiments, the first precursors and the recombinant fusion protein are contacted inside a cell expressing the recombinant fusion protein, e.g., a host cell into which an expression vector encoding the fusion protein has been introduced. The precursors can, e.g., be synthesized in the cell (naturally or by a pathway engineered into the cell for that purpose), provided exogenously and taken up by the cell, or the like. In another class of embodiments, the first precursors and the recombinant fusion protein are contacted in vitro, e.g., using purified recombinant fusion protein, an extract from a cell expressing the fusion protein, or the like. One or more additional enzymes, e.g., required for activity of the fusion protein (e.g., pantetheinyl transferase to attach a phosphopantetheine cofactor to an acyl carrier domain in the fusion protein), are optionally expressed in the cell or provided in the in vitro translation system.

The product can be any of an extremely wide variety of polyketones. As just a few examples, the product can be an aliphatic or linear decarboxylated methylketone, a phloroglucinol, an acyl phloroglucinol, a branched acyl phloroglucinol, a phlorisovalerophenone, a chalcone, an acridone, a bibenzyl, an acyl resorcinol, an acyl resorcinolic acid, an alkyl resorcinol, a stilbene, a stilbene acid, a tetrahydroxynaphthalene, an acyl chromone, an acyl lactone, an acyl pyrone, an olivetol, or an olivitolic acid product. The product is optionally further modified by downstream enzymes that perform glycosylation, hydroxylation, halogenation, prenylation, acylation, alkylation, oxidation, and/or similar steps to convert the polyketide product of the fusion protein into a desired final product. For example, olivetolic acid or olivetol can be further modified to form a cannabinoid natural product, alkylresorcinols can be modified to produce sorgoleone and related allelopathic natural products or anacardic acid and other urushiols, and branched acyl phloroglucinols such as phlorisovalerophenone can be modified to produce bitter acids such as humulone and lupulone.

The polyketide product is optionally purified, using techniques well known in the art. Similarly, established techniques can be used to confirm or determine the identity of the polyketide product, for example, thin layer chromatography or mass spectrometry (e.g., LC-MS-MS).

A wide variety of suitable precursors are well known in the art and others can be readily identified (see, e.g., Austin and Noel (2003) Nat Prod Rep 20:79-110, Moore and Hertweck (2002) “Biosynthesis and attachment of novel bacterial polyketide synthase starter units” Nat Prod Rep 19:70-99, and references herein). As just a few examples, extender units including, but not limited to, malonyl-, methylmalonyl-, ethylmalonyl-, and methoxymalonyl-thioesters (CoA or ACP) and starter units including, but not limited to, thioesters of propionate, isobutyrate, isovalerate, 2-methylbutyrate, other linear or branched fatty acids, and benzoic acid can be utilized. Selection of appropriate precursors to produce a desired product using a fusion protein of the invention is within the ability of one of skill in the art.

The recombinant fusion protein can be any of those described herein. For example, the fusion protein can include one or more of a ketoacyl synthase domain, an acyl transferase domain, a dehydratase domain, an enoyl reductase domain, a ketoreductase domain, and an acyl carrier domain, e.g., two or more, three or more, four or more, five or more, or even six or more such domains. For example, in one class of embodiments, the recombinant fusion protein includes type I fatty acid synthase ketoacyl synthase, acyl transferase, dehydratase, enoyl reductase, ketoreductase, and acyl carrier domains. The type III PKS domain optionally replaces a thioesterase domain in a type I FAS or type I PKS. The recombinant fusion protein optionally includes a type III PKS domain derived from a protein including, but not limited to, chalcone synthase, stilbene synthase, stilbenecarboxylate synthase, bibenzyl synthase, homoeriodictyol/eriodictyol synthase, acridone synthase, benzophenone synthase, phlorisovalerophenone synthase, coumaroyl triacetic acid synthase, benzalacetone synthase, 1,3,6,8-tetrahydroxynaphthalene synthase, phloroglucinol synthase, dihydroxyphenylacetate synthase, alkylresorcinol synthase, alkylpyrone synthase, aloesone synthase, pentaketide chromone synthase, octaketide synthase, the Steely2 C-terminal domain, and benzalacetone synthase. The type III polyketide synthase domain is optionally C-terminal to the at least one type I polyketide synthase domain or type I fatty acid synthase domain in the recombinant fusion protein.

The recombinant fusion protein optionally includes one or more domains derived from the Steely1 or Steely2 proteins described herein (SEQ ID NO:1 and 2, respectively), including conservative variants thereof as well as variants with altered function. For example, the fusion protein optionally includes one or more of a ketoacyl synthase domain, acyl transferase domain, dehydratase domain, enoyl reductase domain, ketoreductase domain, and acyl carrier domain derived from Steely1 or Steely2. In one class of embodiments, the fusion protein includes the Steely1 PKS III domain (approximately residues 2776-3147 of SEQ ID NO:1); the Steely1 PKS III domain and the linker N-terminal to it (approximately residues 2629-3147 of SEQ ID NO:1); the Steely1 AC domain, PKS III domain, and the linker connecting them (approximately residues 2560-3147 of SEQ ID NO:1); or the Steely1 linker connecting the AC and PKS III domains (approximately residues 2629-2775 of SEQ ID NO:1); or an amino acid sequence at least about 25% identical thereto (e.g., at least about 50%, at least about 75%, at least about 90%, at least about 95%, at least about 98%, or at least about 99% identical thereto). In another class of embodiments, the fusion protein includes the Steely2 PKS III domain (approximately residues 2616-2968 of SEQ ID NO:2); the Steely2 PKS III domain and the linker N-terminal to it (approximately residues 2473-2968 of SEQ ID NO:2); the Steely2 AC domain, PKS III domain, and the linker connecting them (approximately residues 2412-2968 of SEQ ID NO:2); or the Steely2 linker connecting the AC and PKS III domains (approximately residues 2473-2615 of SEQ ID NO:2); or an amino acid sequence at least about 25% identical thereto (e.g., at least about 50%, at least about 75%, at least about 90%, at least about 95%, at least about 98%, or at least about 99% identical thereto). Optionally, the fusion protein includes 50 or more contiguous amino acids of SEQ ID NO:1 or SEQ ID NO:2 (e.g., 100 or more, 200 or more, 300 or more, 400 or more, 500 or more, 1000 or more, 1500 or more, 2000 or more, or even 2500 or more), or an amino acid sequence at least about 25% identical thereto (e.g., at least about 50%, at least about 75%, at least about 90%, at least about 95%, at least about 97%, or at least about 99% identical thereto).

### Making Recombinant Fusion Proteins

In one aspect, the invention provides methods of making fusion proteins. For example, one class of embodiments provides methods of making a recombinant fusion protein. In the methods, at least a first DNA molecule encoding at least a first domain and at least a second DNA molecule encoding a second domain are provided. The first DNA molecule is joined (e.g., ligated) in frame with the second DNA molecule to generate a recombinant DNA molecule encoding the fusion protein, and the recombinant DNA molecule is translated to produce the fusion protein. In the resulting fusion protein, the first domain catalyzes conversion of one or more precursors to an intermediate, which intermediate is covalently bound to the fusion protein (e.g., to an AC or PCP domain also encoded by the recombinant DNA molecule), and the second domain catalyzes conversion of the intermediate to a product. The resulting fusion protein can be, e.g., any of those described herein.

One general class of embodiments provides methods of making a fusion protein. In the methods, one or more first DNA molecules collectively encoding one or more type I polyketide synthase or fatty acid synthase domains are provided. At least one second DNA molecule encoding a type III polyketide synthase domain is also provided. The one or more first DNA molecules are joined (e.g., ligated) in frame with the second DNA molecule to generate a recombinant DNA molecule encoding the fusion protein, then the recombinant DNA molecule is translated to produce the fusion protein.

The recombinant DNA molecule is optionally introduced into a host cell, in which it is translated to produce the fusion protein. Alternatively, the recombinant DNA molecule can be translated in vitro, for example. One or more additional enzymes required for activity of the fusion protein (e.g., pantetheinyl transferase to attach a phosphopantetheine cofactor to an acyl carrier domain in the fusion protein) are optionally expressed in the cell or provided in the in vitro translation system if necessary.

Libraries of recombinant DNA molecules are optionally produced and screened to identify fusion proteins(s) possessing a desired activity (e.g., use of a particular precursor and/or production of a particular product). For example, members of a library of different first domains can be joined to a given second domain and the resulting fusion proteins screened. Similarly, a given first domain can be joined to members of a library of different second domains and the resulting fusion proteins screened. As yet another example, members of libraries of first and second domains can be joined and the resulting fusion proteins screened. The libraries can be generated by any of the variety of techniques known in the art, for example, derived from natural sources, by mutagenesis, by DNA shuffling, etc.

Thus, in one embodiment, providing one or more first DNA molecules comprises providing a library of first DNA molecules differing from each other in at least one nucleotide. In a related embodiment, providing at least one second DNA molecule comprises providing a library of second DNA molecules differing from each other in at least one nucleotide. In one class of embodiments, joining the one or more first DNA molecules with the second DNA molecule to generate a recombinant DNA molecule comprises joining one or more first DNA molecules or a library thereof with the second DNA molecule or a library thereof to generate a library of recombinant DNA molecules. The library of recombinant DNA molecules can then be translated to provide a library of fusion proteins, which is screened for a desired property (e.g., by assaying members' ability to produce a desired product, incorporate a desired starter or extender unit, or the like). The recombinant DNA molecule encoding a fusion protein with the desired property is optionally recovered or isolated from the library of recombinant DNA molecules.

As noted above, a library of first DNA molecules, a library of second DNA molecules, and/or the library of recombinant DNA molecules is optionally subjected to DNA shuffling. As an example, a library of first DNA molecules encoding a type I PKS or FAS domain can be shuffled (or multiple libraries of different types of type I domains can be shuffled), while a library of second DNA molecules encoding a type III PKS domain is also shuffled; the two libraries can then be ligated together, followed by selection for fusion proteins with the desired property as described above. As another example, a library of first DNA molecules encoding a type I PKS or FAS domain can be ligated to a library of second DNA molecules encoding a type III PKS domain, then the resulting library can be shuffled. DNA shuffling is described in greater detail in Cohen (2001) “How DNA shuffling works” Science 293:237, U.S. patent application publications 20030027156 “Methods and compositions for polypeptide engineering,” 20010044111 “Method for generating recombinant DNA molecules in complex mixtures,” and 20020132308 “Novel constructs and their use in metabolic pathway engineering,” and references herein.

Generally, nucleic acids encoding a fusion protein of the invention can be made by cloning, recombination, in vitro synthesis, in vitro amplification and/or other available methods. In addition, a variety of recombinant methods can be used for expressing an expression vector that encodes a fusion protein of the invention. Recombinant methods for making nucleic acids, expression, and optional isolation of expressed products are well known and are described, e.g., in Sambrook et al., Molecular Cloning—A Laboratory Manual (3rd Ed.), Vol. 1-3, Cold Spring Harbor Laboratory, Cold Spring Harbor, N.Y., 2000 (“Sambrook”), Current Protocols in Molecular Biology, F. M. Ausubel et al., eds., Current Protocols, a joint venture between Greene Publishing Associates, Inc. and John Wiley & Sons, Inc. (supplemented through 2007) (“Ausubel”), and Innis et al. (eds.), PCR Protocols: A Guide to Methods and Applications, Academic Press Inc., San Diego, Calif. (1990) (“Innis”). In addition, essentially any nucleic acid can be custom or standard ordered from any of a variety of commercial sources, such as Operon Technologies Inc. (Alameda, Calif.). Optionally, techniques that facilitate synthesis of long nucleotide sequences are employed; see, e.g., Kodumal et al. (2004) supra.

Various types of mutagenesis are optionally used in the present invention, e.g., to introduce convenient restriction sites or to modify specificities of type I FAS or PKS or type III PKS domains, e.g., as discussed above. In general, any available mutagenesis procedure can be used for making such mutants. Such mutagenesis procedures optionally include selection of mutant nucleic acids and polypeptides for one or more activity of interest (e.g., altered starter or extender unit or product specificity). Procedures that can be used include, but are not limited to: site-directed point mutagenesis, random point mutagenesis, in vitro or in vivo homologous recombination (DNA shuffling), mutagenesis using uracil containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, degenerate PCR, double-strand break repair, and many others known to persons of skill.

Optionally, mutagenesis can be guided by known information from a naturally occurring fatty acid or polyketide synthase or a domain thereof, or of a known altered or mutated synthase, e.g., sequence, sequence comparisons, physical properties, crystal structure and/or the like as discussed above. However, in another class of embodiments, modification can be essentially random (e.g., as in classical DNA shuffling).

Additional information on mutation formats is found in, for example, Sambrook, Ausubel, and Innis. The following publications and references cited within provide still additional detail on mutation formats: Arnold, Protein engineering for unusual environments, Current Opinion in Biotechnology 4:450-455 (1993); Bass et al., Mutant Trp repressors with new DNA-binding specificities, Science 242:240-245 (1988); Botstein & Shortle, Strategies and applications of in vitro mutagenesis, Science 229:1193-1201 (1985); Carter et al., Improved oligonucleotide site-directed mutagenesis using M13 vectors, Nucl. Acids Res. 13: 4431-4443 (1985); Carter, Site-directed mutagenesis, Biochem. J. 237:1-7 (1986); Carter, Improved oligonucleotide-directed mutagenesis using M13 vectors, Methods in Enzymol. 154: 382-403 (1987); Dale et al., Oligonucleotide-directed random mutagenesis using the phosphorothioate method, Methods Mol. Biol. 57:369-374 (1996); Eghtedarzadeh & Henikoff, Use of oligonucleotides to generate large deletions, Nucl. Acids Res. 14: 5115 (1986); Fritz et al., Oligonucleotide-directed construction of mutations: a gapped duplex DNA procedure without enzymatic reactions in vitro, Nucl. Acids Res. 16: 6987-6999 (1988); Grundström et al., Oligonucleotide-directed mutagenesis by microscale ‘shot-gun’ gene synthesis, Nucl. Acids Res. 13: 3305-3316 (1985); Kunkel, The efficiency of oligonucleotide directed mutagenesis, in Nucleic Acids & Molecular Biology (Eckstein, F. and Lilley, D. M. J. eds., Springer Verlag, Berlin)) (1987); Kunkel, Rapid and efficient site-specific mutagenesis without phenotypic selection, Proc. Natl. Acad. Sci. USA 82:488-492 (1985); Kunkel et al., Rapid and efficient site-specific mutagenesis without phenotypic selection, Methods in Enzymol. 154, 367-382 (1987); Kramer et al., The gapped duplex DNA approach to oligonucleotide-directed mutation construction, Nucl. Acids Res. 12: 9441-9456 (1984); Kramer & Fritz Oligonucleotide-directed construction of mutations via gapped duplex DNA, Methods in Enzymol. 154:350-367 (1987); Kramer et al., Point Mismatch Repair, Cell 38:879-887 (1984); Kramer et al., Improved enzymatic in vitro reactions in the gapped duplex DNA approach to oligonucleotide-directed construction of mutations, Nucl. Acids Res. 16: 7207 (1988); Ling et al., Approaches to DNA mutagenesis: an overview, Anal Biochem. 254(2): 157-178 (1997); Lorimer and Pastan Nucleic Acids Res. 23, 3067-8 (1995); Mandecki, Oligonucleotide-directed double-strand break repair in plasmids of Escherichia coli: a method for site-specific mutagenesis, Proc. Natl. Acad. Sci. USA, 83:7177-7181 (1986); Nakamaye & Eckstein, Inhibition of restriction endonuclease Nci I cleavage by phosphorothioate groups and its application to oligonucleotide-directed mutagenesis, Nucl. Acids Res. 14: 9679-9698 (1986); Nambiar et al., Total synthesis and cloning of a gene coding for the ribonuclease S protein, Science 223: 1299-1301 (1984); Sakamar and Khorana, Total synthesis and expression of a gene for the a-subunit of bovine rod outer segment guanine nucleotide-binding protein (transducin), Nucl. Acids Res. 14: 6361-6372 (1988); Sayers et al., Y-T Exonucleases in phosphorothioate-based oligonucleotide-directed mutagenesis, Nucl. Acids Res. 16:791-802 (1988); Sayers et al., Strand specific cleavage of phosphorothioate-containing DNA by reaction with restriction endonucleases in the presence of ethidium bromide, (1988) Nucl. Acids Res. 16: 803-814; Sieber, et al., Nature Biotechnology, 19:456-460 (2001); Smith, In vitro mutagenesis, Ann. Rev. Genet. 19:423-462 (1985); Methods in Enzymol. 100: 468-500 (1983); Methods in Enzymol. 154: 329-350 (1987); Stemmer, Nature 370, 389-91 (1994); Taylor et al., The use of phosphorothioate-modified DNA in restriction enzyme reactions to prepare nicked DNA, Nucl. Acids Res. 13: 8749-8764 (1985); Taylor et al., The rapid generation of oligonucleotide-directed mutations at high frequency using phosphorothioate-modified DNA, Nucl. Acids Res. 13: 8765-8787 (1985); Wells et al., Importance of hydrogen-bond formation in stabilizing the transition state of subtilisin, Phil. Trans. R. Soc. Lond. A 317: 415-423 (1986); Wells et al., Cassette mutagenesis: an efficient method for generation of multiple mutations at defined sites, Gene 34:315-323 (1985); Zoller & Smith, Oligonucleotide-directed mutagenesis using M13-derived vectors: an efficient and general procedure for the production of point mutations in any DNA fragment, Nucleic Acids Res. 10:6487-6500 (1982); Zoller & Smith, Oligonucleotide-directed mutagenesis of DNA fragments cloned into M13 vectors, Methods in Enzymol. 100:468-500 (1983); and Zoller & Smith, Oligonucleotide-directed mutagenesis: a simple method using two oligonucleotide primers and a single-stranded DNA template, Methods in Enzymol. 154:329-350 (1987). Additional details on many of the above methods can be found in Methods in Enzymology Volume 154, which also describes useful controls for trouble-shooting problems with various mutagenesis methods. A variety of kits for performing mutagenesis are commercially available (see, e.g., the QuikChange® site-directed mutagenesis kit from Stratagene and the BD Transformer™ site-directed mutagenesis kit from Clontech).

In addition, a plethora of kits are commercially available for the purification of plasmids or other relevant nucleic acids from cells, (see, e.g., EasyPrep™, FlexiPrep™, both from Pharmacia Biotech; StrataClean™, from Stratagene; and, QIAprep™ from Qiagen). Any isolated and/or purified nucleic acid can be further manipulated to produce other nucleic acids, used to transfect cells, incorporated into related vectors to infect organisms for expression, and/or the like. Typical cloning vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for either or both prokaryotic and eukaryotic systems. Vectors are suitable for replication and integration in prokaryotes, eukaryotes, or both. See, Giliman & Smith, Gene 8:81 (1979); Roberts, et al., Nature, 328:731 (1987); Schneider, B., et al., Protein Expr. Purif. 6435:10 (1995); Ausubel; Sambrook; and Berger and Kimmel, Guide to Molecular Cloning Techniques, Methods in Enzymology volume 152 Academic Press, Inc., San Diego, Calif. A large number of suitable vectors are known in the art and/or commercially available. A catalogue of bacteria and bacteriophages useful for cloning is provided, e.g., by the American Type Culture Collection (ATCC), e.g., The ATCC Catalogue of Bacteria and Bacteriophage published yearly by the ATCC. Additional basic procedures for sequencing, cloning and other aspects of molecular biology and underlying theoretical considerations are also found in Watson et al. (1992) Recombinant DNA Second Edition, Scientific American Books, NY.

Other useful references, e.g. for cell isolation and culture (e.g., for subsequent nucleic acid or polypeptide isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

A variety of protein isolation and detection methods are known and can be used to isolate polypeptides, e.g., from recombinant cultures of cells expressing the recombinant fusion proteins of the invention where such purification is desired. A variety of protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2_{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000). The fusion protein optionally includes a tag to facilitate purification, e.g., a GST, polyhistidine, and/or S tag. The tag(s) are optionally removed by digestion with an appropriate protease (e.g., thrombin or enterokinase).

### Heterologous Expression Systems

In one aspect, the invention provides a cell in which a fusion protein (e.g., a recombinant fusion protein) of the invention is heterologously expressed. For example, one class of embodiments provides a cell comprising an expression vector that includes a promoter operably linked to a polynucleotide encoding a fusion protein, e.g., a recombinant fusion protein, which fusion protein comprises at least one type I polyketide or fatty acid synthase domain and a type III polyketide synthase domain. The expression vector can be introduced into the cell by any of the variety of techniques well known in the art, including, e.g., electroporation, calcium phosphate precipitation, lipid mediated transfection (lipofection), biolistic delivery, or the like. Expression is optionally constitutive or inducible, as desired. The cell is optionally used for in vivo synthesis of a polyketide (or other product) produced by action of the expressed fusion protein. In other embodiments, an extract or lysate from the cell is used for in vitro production of the polyketide (or other product). In still other embodiments, the fusion protein is purified from the cell.

The host cell is optionally one that does not naturally produce polyketides, such as E. coli. One or more additional enzymes required for activity of the fusion protein are optionally expressed in the cell, endogenously or heterologously. For example, pantetheinyl transferase can be heterologously expressed in E coli to attach a phosphopantetheine cofactor to an acyl carrier domain in the fusion protein; see, e.g., Pfeifer et al. (2001) “Biosynthesis of complex polyketides in a metabolically engineered strain of E. coli” Science 291:1790-1792. Exemplary host cells also include PKS gene modified (or knockout) versions of natural hosts such as Dictyostelium. Exemplary host cells include, but are not limited to, prokaryotic cells such as E. coli and other bacteria and eukaryotic cells such as yeast, plant, insect, amphibian, avian, and mammalian cells, including human cells. Bacteria with a higher or lower AT vs. GC content in their genomes relative to E. coli are optionally used as host cells, to optimize expression of similarly-biased genes; for example, S. coelicolor or S. lividans is optionally used for expression of GC-rich constructs (Anne and Van Mellaert (1993) “Streptomyces lividans as host for heterologous protein production” FEMS Microbiol Lett. 114(2):121-8), e.g., fusion proteins including PKSs from other Streptomyces species, while Pseudomonas species are optionally used for expression of AT-rich constructs.

Where in vivo production of polyketide (or other) product by the fusion protein is desired, the precursors required for polyketide (or other) synthesis (e.g., suitable starter and extender units, natural or unnatural D- or L-amino acids, etc.) can be endogenous to the cell, such precursors can be provided exogenously and taken up by the cell, and/or biosynthetic pathway(s) to create the precursors in vivo can be generated in the host cell. For example, biosynthetic pathways for starter and/or extender units are optionally generated in the host cell by adding new enzymes or modifying existing host cell pathways. See, e.g., Pfeifer et al. (2001) supra, in which a pathway for methylmalonyl-CoA biosynthesis was introduced into E. coli. Pfeifer et al. also describe a technique for increasing the cellular pool of a starter unit, propionyl-CoA, by disrupting a propionate catabolic pathway.

A host cell expressing a fusion protein for production of polyketide also optionally expresses one or more additional enzymes, for example, enzymes whose collective action converts a polyketide product of the fusion protein into a final product. Such downstream tailoring enzymes can perform glycosylation, hydroxylation, halogenation, prenylation, acylation, alkylation, oxidation, and/or similar steps as necessary to produce the desired final product. Any such downstream enzymes can be expressed endogenously and/or heterologously.

Additional new enzymes expressed in the host cell (e.g., for fusion protein activity, precursor synthesis, and/or downstream tailoring enzymes) are optionally naturally occurring enzymes, e.g., from other species, or artificially evolved enzymes. The genes for these enzymes can be introduced into a cell by transforming the cell with a plasmid comprising the genes and/or integrating the genes into the host's genome. The genes, when expressed in the cell, provide an enzymatic pathway to synthesize the desired compound. Examples of the types of enzymes that are optionally added are provided herein, and additional enzyme sequences can be found, e.g., in Genbank and in the literature.

Where artificially evolved enzymes are added into the cell, any of a variety of methods can be used for producing novel enzymes, e.g., for use in biosynthetic pathways or for evolution of existing pathways, in vitro or in vivo. Many available methods of evolving enzymes and other biosynthetic pathway components can be applied to the present invention to produce precursors or products (or, indeed, to evolve synthases or domains thereof to have new substrate specificities or other activities of interest). For example, DNA shuffling is optionally used to develop novel enzymes and/or pathways of such enzymes for the production of precursors or products (or production of new synthases), in vitro or in vivo. See, e.g., Stemmer (1994) “Rapid evolution of a protein in vitro by DNA shuffling” Nature 370(4):389-391; and, Stemmer, (1994) “DNA shuffling by random fragmentation and reassembly: In vitro recombination for molecular evolution” Proc. Natl. Acad. Sci. USA., 91:10747-10751. A related approach shuffles families of related (e.g., homologous) genes to quickly evolve enzymes with desired characteristics. An example of such “family gene shuffling” methods is found in Crameri et al. (1998) “DNA shuffling of a family of genes from diverse species accelerates directed evolution” Nature, 391(6664):288-291. New enzymes (whether biosynthetic pathway components or synthetases) can also be generated using a DNA recombination procedure known as “incremental truncation for the creation of hybrid enzymes” (“ITCHY”), e.g., as described in Ostermeier et al. (1999) “A combinatorial approach to hybrid enzymes independent of DNA homology” Nature Biotech 17:1205. This approach can also be used to generate a library of enzyme or other pathway variants which can serve as substrates for one or more in vitro or in vivo recombination methods. See, also, Ostermeier et al. (1999) “Combinatorial Protein Engineering by Incremental Truncation” Proc. Natl. Acad. Sci. USA 96: 3562-67, and Ostermeier et al. (1999), “Incremental Truncation as a Strategy in the Engineering of Novel Biocatalysts” Biological and Medicinal Chemistry 7:2139-44. Another approach uses exponential ensemble mutagenesis to produce libraries of enzyme or other pathway variants that are, e.g., selected for an ability to catalyze a biosynthetic reaction relevant to producing a precursor or product (or a new synthase). In this approach, small groups of residues in a sequence of interest are randomized in parallel to identify, at each altered position, amino acids which lead to functional proteins. Examples of such procedures, which can be adapted to the present invention to produce new enzymes for the production of precursors or products (or new synthases) are found in Delegrave and Youvan (1993) Biotechnology Research 11:1548-1552. In yet another approach, random or semi-random mutagenesis using doped or degenerate oligonucleotides for enzyme and/or pathway component engineering can be used, e.g., by using the general mutagenesis methods of e.g., Arkin and Youvan (1992) “Optimizing nucleotide mixtures to encode specific subsets of amino acids for semi-random mutagenesis” Biotechnology 10:297-300; or Reidhaar-Olson et al. (1991) “Random mutagenesis of protein sequences using oligonucleotide cassettes” Methods Enzymol. 208:564-86. Yet another approach, often termed a “non-stochastic” mutagenesis, which uses polynucleotide reassembly and site-saturation mutagenesis can be used to produce enzymes and/or pathway components, which can then be screened for an ability to perform one or more synthase or biosynthetic pathway function (e.g., for the production of precursors or products in vivo). See, e.g., Short “Non-Stochastic Generation of Genetic Vaccines and Enzymes” WO 00/46344.

An alternative to such mutational methods involves recombining entire genomes of organisms and selecting resulting progeny for particular pathway functions (often referred to as “whole genome shuffling”). This approach can be applied to the present invention, e.g., by genomic recombination and selection of an organism (e.g., an E. coli or other cell) for an ability to produce a desired precursor or product (or intermediate thereof). For example, methods taught in the following publications can be applied to pathway design for the evolution of existing and/or new pathways in cells to produce precursors or products in vivo: Patnaik et al. (2002) “Genome shuffling of lactobacillus for improved acid tolerance” Nature Biotechnology 20(7):707-712; and Zhang et al. (2002) “Genome shuffling leads to rapid phenotypic improvement in bacteria” Nature 415:644-646.

Other techniques for organism and metabolic pathway engineering, e.g., for the production of desired compounds, are also available and can also be applied to the production of precursors or products. Examples of publications teaching useful pathway engineering approaches include: Nakamura and White (2003) “Metabolic engineering for the microbial production of 1,3 propanediol” Curr. Opin. Biotechnol. 14(5):454-9; Berry et al. (2002) “Application of Metabolic Engineering to improve both the production and use of Biotech Indigo” J. Industrial Microbiology and Biotechnology 28:127-133; Banta et al. (2002) “Optimizing an artificial metabolic pathway: Engineering the cofactor specificity of Corynebacterium 2,5-diketo-D-gluconic acid reductase for use in vitamin C biosynthesis” Biochemistry 41(20):6226-36; Selivonova et al. (2001) “Rapid Evolution of Novel Traits in Microorganisms” Applied and Environmental Microbiology 67:3645, and many others.

Regardless of the method used, typically, the precursor(s) produced with an engineered biosynthetic pathway of the invention is produced in a concentration sufficient for efficient polyketide (or other product) biosynthesis, e.g., a natural cellular amount, but not to such a degree as to significantly affect the concentration of other cellular compounds or to exhaust cellular resources. Once a cell is engineered to produce enzymes desired for a specific pathway and a precursor is generated, in vivo selections are optionally used to further optimize the production of the precursor for both polyketide (or other product) synthesis and cell growth.

### Nucleic Acid and Polypeptide Sequences and Variants

Sequences for a variety of naturally occurring and recombinant type I FAS, type I PKS, NRPS, type III PKS, type II PKS, KAS III, HMG-CoA synthetases, beta-ketoacyl CoA synthases, and related proteins (including sequences of various domains or modules as well as full-length proteins) and nucleic acids are publicly available. See, for example, the references herein. In addition, sequences of two novel, naturally occurring type I-type III fusion proteins from Dictyostelium discoideum, Steely1 and Steely2, are described herein. The amino acid sequence of Steely1 is presented as SEQ ID NO:1 and the corresponding nucleotide sequence as SEQ ID NO:3 (Table 3). The amino acid sequence of Steely2 is presented as SEQ ID NO:2 and the corresponding nucleotide sequence as SEQ ID NO:4 (Table 3). These sequences, as well as corresponding genomic sequences, are also available at dictyBase (dictybase (dot) org) under accession numbers DDB0190208 and DDB0219613. A number of additional, novel polypeptides are described herein, including recombinant type I FAS/PKS-type III PKS fusion proteins.

In one aspect, the invention provides a variety of polynucleotides encoding the novel polypeptides of the invention, e.g., the novel fusion proteins. For example, one class of embodiments provides a polynucleotide that encodes a recombinant fusion protein, wherein the fusion protein comprises a first domain that catalyzes conversion of one or more precursors to an intermediate, which intermediate is covalently bound to the fusion protein, and a second domain that catalyzes conversion of the intermediate to a product. The recombinant fusion protein can be any of those described herein. A related class of embodiments provides a polynucleotide that encodes a recombinant fusion protein, wherein the fusion protein comprises at least one type I polyketide or fatty acid synthase domain and a type III polyketide synthase domain. Again, the recombinant fusion protein can be any of those described herein. For example, the recombinant fusion protein can include one or more domains selected from a type I PKS or FAS ketoacyl synthase domain, acyl transferase domain, dehydratase domain, enoyl reductase domain, ketoreductase domain, and acyl carrier domain. The type III polyketide synthase domain is optionally C-terminal to the at least one type I polyketide synthase domain or type I fatty acid synthase domain, e.g., replacing a C-terminal TE domain in a type I PKS or FAS polypeptide. As for the embodiments above, the fusion protein optionally includes one or more linker and/or domain sequences from Steely1 or Steely2. The polynucleotide optionally constitutes one member of a library of polynucleotides, e.g., polynucleotides differing by at least one nucleotide and encoding different recombinant fusion proteins.

One of skill will appreciate that the invention provides many related sequences with the functions described herein, for example, polynucleotides encoding fusion proteins. Because of the degeneracy of the genetic code, many polynucleotides equivalently encode a given polypeptide sequence. Polynucleotide sequences complementary to any of the above described sequences are included among the polynucleotides of the invention. Similarly, an artificial or recombinant nucleic acid that hybridizes to a polynucleotide indicated above under highly stringent conditions over substantially the entire length of the nucleic acid (and is other than a naturally occurring polynucleotide) is a polynucleotide of the invention.

In certain embodiments, a vector (e.g., a plasmid, a cosmid, a phage, a virus, etc.) comprises a polynucleotide of the invention. In one embodiment, the vector is an expression vector. In a related embodiment, the expression vector includes a promoter operably linked to one or more of the polynucleotides of the invention. In another embodiment, a cell comprises a vector (e.g., an expression vector) that includes a polynucleotide of the invention.

One of skill will also appreciate that many variants of the disclosed sequences are included in the invention. For example, conservative variations of the disclosed sequences that yield a functionally similar sequence are included in the invention. Variants of the nucleic acid polynucleotide sequences, wherein the variants hybridize to at least one disclosed sequence, are considered to be included in the invention. Unique subsequences of the sequences disclosed herein, as determined by, e.g., standard sequence comparison techniques, are also included in the invention.

Conservative Variations

Owing to the degeneracy of the genetic code, “silent substitutions” (i.e., substitutions in a nucleic acid sequence which do not result in an alteration in an encoded polypeptide) are an implied feature of every nucleic acid sequence that encodes an amino acid sequence. Similarly, “conservative amino acid substitutions,” where one or a limited number of amino acids in an amino acid sequence are substituted with different amino acids with highly similar properties, are also readily identified as being highly similar to a disclosed construct. Such conservative variations of each disclosed sequence are a feature of the present invention.

“Conservative variations” of a particular nucleic acid sequence refers to those nucleic acids which encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid, while retaining the relevant function of the polypeptide such as enzymatic activity (for example, the conservative substitution can be of a residue distal to the active site region). Thus, “conservative variations” of a listed polypeptide sequence of the present invention include substitutions of a small percentage, typically less than 5%, more typically less than 2% or 1%, of the amino acids of the polypeptide sequence, with an amino acid of the same conservative substitution group. Finally, the addition of sequences which do not alter the encoded activity of a nucleic acid molecule, such as the addition of a non-functional or tagging sequence (introns in the nucleic acid, poly His or similar sequences in the encoded polypeptide, etc.), is a conservative variation of the basic nucleic acid or polypeptide.

Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the polypeptide molecule. The following sets forth example groups that contain natural amino acids of like chemical properties, where substitutions within a group is a “conservative substitution”. It will be evident that a variety of similar tables exist in the art, and that conservative vs. non-conservative substitutions can be classified, e.g., based on steric bulk and/or hydropathy (e.g., taking into account the Kyte/Doolittle hydropathy index and/or structural statistics comparing trends (solvent-exposed or buried) observed in proteins for each residue.

TABLE 1
Conservative Amino Acid Substitutions

| | | | Positively | Negatively |
|---|---|---|---|---|
| Nonpolar and/or | Polar, | | Charged | Charged |
| Aliphatic Side | Uncharged | Aromatic Side | Side | Side |
| Chains | Side Chains | Chains | Chains | Chains |
| Glycine | Serine | Phenylalanine | Lysine | Aspartate |
| Alanine | Threonine | Tyrosine | Arginine | Glutamate |
| Valine | Cysteine | Tryptophan | Histidine | |
| Leucine | Methionine | | | |
| Isoleucine | Asparagine | | | |
| Proline | Glutamine | | | |

Nucleic Acid Hybridization

Comparative hybridization can be used to identify nucleic acids of the invention, including conservative variations of nucleic acids of the invention. In addition, target nucleic acids which hybridize to a nucleic acid of the invention under high, ultra-high and ultra-ultra high stringency conditions, where the nucleic acids are other than a naturally occurring nucleic acid, are a feature of the invention. Examples of such nucleic acids include those with one or a few silent or conservative nucleic acid substitutions as compared to a given nucleic acid sequence of the invention.

A test nucleic acid is said to specifically hybridize to a probe nucleic acid when it hybridizes at least 50% as well to the probe as to the perfectly matched complementary target, i.e., with a signal to noise ratio at least half as high as hybridization of the probe to the target under conditions in which the perfectly matched probe binds to the perfectly matched complementary target with a signal to noise ratio that is at least about 5×-10× as high as that observed for hybridization to any of the unmatched target nucleic acids.

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, N.Y.), as well as in Ausubel; Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis, labeling, detection and quantification of DNA and RNA, including oligonucleotides.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of stringent wash conditions is a 0.2×SSC wash at 65° C. for 15 minutes (see, Sambrook et al., Molecular Cloning—A Laboratory Manual (3rd Ed.), Vol. 1-3, Cold Spring Harbor Laboratory, Cold Spring Harbor, N.Y., 2000 for a description of SSC buffer). Often the high stringency wash is preceded by a low stringency wash to remove background probe signal. An example low stringency wash is 2×SSC at 40° C. for 15 minutes. In general, a signal to noise ratio of 5× (or higher) than that observed for an unrelated probe in the particular hybridization assay indicates detection of a specific hybridization.

“Stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993), supra and in Hames and Higgins, 1 and 2. Stringent hybridization and wash conditions can easily be determined empirically for any test nucleic acid. For example, in determining stringent hybridization and wash conditions, the hybridization and wash conditions are gradually increased (e.g., by increasing temperature, decreasing salt concentration, increasing detergent concentration and/or increasing the concentration of organic solvents such as formalin in the hybridization or wash), until a selected set of criteria are met. For example, in highly stringent hybridization and wash conditions, the hybridization and wash conditions are gradually increased until a probe binds to a perfectly matched complementary target with a signal to noise ratio that is at least 5× as high as that observed for hybridization of the probe to an unmatched target.

“Very stringent” conditions are selected to be equal to the thermal melting point (Tₘ) for a particular probe. The Tₘ is the temperature (under defined ionic strength and pH) at which 50% of the test sequence hybridizes to a perfectly matched probe. For the purposes of the present invention, generally, “highly stringent” hybridization and wash conditions are selected to be about 5° C. lower than the Tₘ, for the specific sequence at a defined ionic strength and pH.

“Ultra high-stringency” hybridization and wash conditions are those in which the stringency of hybridization and wash conditions are increased until the signal to noise ratio for binding of the probe to the perfectly matched complementary target nucleic acid is at least 10× as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or 500× or more as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-ultra-high stringency conditions.

Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code.

Sequence Comparison, Identity, and Homology

The terms “identical” or “percent identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection.

The phrase “substantially identical,” in the context of two nucleic acids or polypeptides (e.g., DNAs encoding a FAS, PKS, fusion protein, or domain thereof, or the amino acid sequence of a FAS, PKS, fusion protein, or domain thereof) refers to two or more sequences or subsequences that have at least about 60%, about 80%, about 90-95%, about 98%, about 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, or over the full length of the two sequences to be compared.

Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity (e.g., identity) over 50, 100, 150 or more residues (nucleotides or amino acids) is routinely used to establish homology (e.g., over the full length of the two sequences to be compared). Higher levels of sequence similarity (e.g., identity), e.g., 30%, 40%, 50%, 60%, 70%, 80%, 90%, 95%, or 99% or more, can also be used to establish homology. Methods for determining sequence similarity percentages (e.g., BLASTP and BLASTN using default parameters) are described herein and are generally available.

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel).

One example of an algorithm that is suitable for determining percent sequence identity and sequence similarity is the BLAST algorithm, which is described in AltSchul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### Structure-Based Design of Recombinant Proteins

Structural data for a polyketide or fatty acid synthase, or a domain thereof, can be used to conveniently identify amino acid residues as candidates for mutagenesis to create recombinant synthases having modified specificities. For example, redesign of a chalcone synthase to possess stilbene synthase or 2-pyrone synthase activity was described above. Similarly, structural data for a synthase or domain thereof can assist in design of fusion proteins, for example, identification of suitable sites at which a type III PKS domain can be joined to a type I PKS or FAS domain. (While the following discussion is couched in terms of design of type I PKS or FAS-type III PKS fusion proteins, it will be evident that similar considerations apply to design of the other fusion proteins of the invention as well.)

The three-dimensional structures of a number of type III PKS and type I PKS and FAS domains have been determined by x-ray crystallography. Several such structures are described herein, and a number of such structures are freely available for download from the Protein Data Bank, at www (dot) rcsb (dot) org/pdb. Structures, along with domain and homology information, are also freely available for search and download from the National Center for Biotechnology Information's Molecular Modeling DataBase, at www (dot) ncbi (dot) nlm (dot) nih (dot) gov/Structure/MMDB/mmdb (dot) shtml. The structures of additional synthases or domains can be modeled, for example, based on homology of the polypeptides with synthases or domains whose structures have already been determined. Alternatively, the structure of a given synthase or domain can be determined by x-ray crystallography or nuclear magnetic resonance (NMR) spectroscopy.

Techniques for crystal structure determination are well known. See, for example, McPherson (1999) Crystallization of Biological Macromolecules Cold Spring Harbor Laboratory; Bergfors (1999) Protein Crystallization International University Line; Mullin (1993) Crystallization Butterwoth-Heinemann; Stout and Jensen (1989) X-ray structure determination: a practical guide, 2nd Edition Wiley Publishers, New York; Ladd and Palmer (1993) Structure determination by X-ray crystallography, 3rd Edition Plenum Press, NewYork; Blundell and Johnson (1976) Protein Crystallography Academic Press, New York; Glusker and Trueblood (1985) Crystal structure analysis: A primer, 2nd Ed. Oxford University Press, NewYork; International Tables for Crystallography, Vol. F. Crystallography of Biological Macromolecules; McPherson (2002) Introduction to Macromolecular Crystallography Wiley-Liss; McRee and David (1999) Practical Protein Crystallography, Second Edition Academic Press; Drenth (1999) Principles of Protein X-Ray Crystallography (Springer Advanced Texts in Chemistry) Springer-Verlag; Fanchon and Hendrickson (1991) Chapter 15 of Crystallographic Computing, Volume 5 IUCr/Oxford University Press; Murthy (1996) Chapter 5 of Crystallographic Methods and Protocols Humana Press; Dauter et al. (2000) “Novel approach to phasing proteins: derivatization by short cryo-soaking with halides” Acta Cryst.D56:232-237; Dauter (2002) “New approaches to high-throughput phasing” Curr. Opin. Structural Biol. 12:674-678; Chen et al. (1991) “Crystal structure of a bovine neurophysin-II dipeptide complex at 2.8 Å determined from the single-wavelength anomalous scattering signal of an incorporated iodine atom” Proc. Natl. Acad. Sci. USA, 88:4240-4244; and Gavira et al. (2002) “Ab initio crystallographic structure determination of insulin from protein to electron density without crystal handling” Acta Cryst.D58:1147-1154.

In addition, a variety of programs to facilitate data collection, phase determination, model building and refinement, and the like are publicly available. Examples include, but are not limited to, the HKL2000 package (Otwinowski and Minor (1997) “Processing of X-ray Diffraction Data Collected in Oscillation Mode” Methods in Enzymology 276:307-326), the CCP4 package (Collaborative Computational Project (1994) “The CCP4 suite: programs for protein crystallography” Acta Crystallogr D 50:760-763), SOLVE and RESOLVE (Terwilliger and Berendzen (1999) Acta Crystallogr D 55 (Pt 4):849-861), SHELXS and SHELXD (Schneider and Sheldrick (2002) “Substructure solution with SHELXD” Acta Crystallogr D Biol Crystallogr 58:1772-1779), Refmac5 (Murshudov et al. (1997) “Refinement of Macromolecular Structures by the Maximum-Likelihood Method” Acta Crystallogr D 53:240-255), PRODRG (van Aalten et al. (1996) “PRODRG, a program for generating molecular topologies and unique molecular descriptors from coordinates of small molecules” J Comput Aided Mol Des 10:255-262), and O (Jones et al. (1991) “Improved methods for building protein models in electron density maps and the location of errors in these models” Acta Crystallogr A 47 (Pt 2):110-119).

Techniques for structure determination by NMR spectroscopy are similarly well described in the literature. See, e.g., Cavanagh et al. (1995) Protein NMR Spectroscopy: Principles and Practice, Academic Press; Levitt (2001) Spin Dynamics: Basics of Nuclear Magnetic Resonance, John Wiley & Sons; Evans (1995) Biomolecular NMR Spectroscopy, Oxford University Press; Wüthrich (1986) NMR of Proteins and Nucleic Acids (Baker Lecture Series), Kurt Wiley-Interscience; Neuhaus and Williamson (2000) The Nuclear Overhauser Effect in Structural and Conformational Analysis, 2nd Edition, Wiley-VCH; Macomber (1998) A Complete Introduction to Modern NMR Spectroscopy, Wiley-Interscience; Downing (2004) Protein NMR Techniques (Methods in Molecular Biology), 2nd edition, Humana Press; Clore and Gronenborn (1994) NMR of Proteins (Topics in Molecular and Structural Biology), CRC Press; Reid (1997) Protein NMR Techniques, Humana Press; Krishna and Berliner (2003) Protein NMR for the Millenium (Biological Magnetic Resonance), Kluwer Academic Publishers; Kiihne and De Groot (2001) Perspectives on Solid State NMR in Biology (Focus on Structural Biology, 1), Kluwer Academic Publishers; Jones et al. (1993) Spectroscopic Methods and Analyses: NMR, Mass Spectrometry, and Related Techniques (Methods in Molecular Biology, Vol. 17), Humana Press; Goto and Kay (2000) Curr. Opin. Struct. Biol. 10:585; Gardner (1998) Annu. Rev. Biophys. Biomol. Struct. 27:357; Wüthrich (2003) Angew. Chem. Int. Ed. 42:3340; Bax (1994) Curr. Opin. Struct. Biol. 4:738; Pervushin et al. (1997) Proc. Natl. Acad. Sci. U.S.A. 94:12366; Fiaux et al. (2002) Nature 418:207; Fernandez and Wider (2003) Curr. Opin. Struct. Biol. 13:570; Ellman et al. (1992) J. Am. Chem. Soc. 114:7959; Wider (2000) BioTechniques 29:1278-1294; Pellecchia et al. (2002) Nature Rev. Drug Discov. (2002) 1:211-219; Arora and Tamm (2001) Curr. Opin. Struct. Biol. 11:540-547; Flaux et al. (2002) Nature 418:207-211; Pellecchia et al. (2001) J. Am. Chem. Soc. 123:4633-4634; and Pervushin et al. (1997) Proc. Natl. Acad. Sci. USA 94:12366-12371.

The structure of a synthase or domain thereof can, as noted, be directly determined or modeled based on the structure of another synthase or domain. The active site region of the synthase or domain can be identified, for example, by homology with other synthases, biochemical analysis of mutant synthases, and/or the like. If desired, the position of a precursor, intermediate, or product in the active site can be modeled. Such modeling can involve simple visual inspection of a model of the synthase or domain, for example, using molecular graphics software such as the PyMOL viewer (open source, freely available at www (dot) pymol (dot) org) or Insight II (commercially available from Accelrys at (www (dot) accelrys (dot) com/products/insight). Alternatively, modeling of the precursor, intermediate, or product in the active site of the synthase or domain or a putative mutant thereof, for example, can involve computer-assisted docking, molecular dynamics, free energy minimization, and/or like calculations. Such modeling techniques have been well described in the literature; see, e.g., Babine and Abdel-Meguid (eds.) (2004) Protein Crystallography in Drug Design, Wiley-VCH, Weinheim; Lyne (2002) “Structure-based virtual screening: An overview” Drug Discov. Today 7:1047-1055; Molecular Modeling for Beginners, at www (dot) usm (dot) maine (dot) edu/˜rhodes/SPVTut/index (dot) html; and Methods for Protein Simulations and Drug Design at www (dot) dddc (dot) ac (dot) cn/embo04; and references therein. Software to facilitate such modeling is widely available, for example, the CHARMm simulation package, available academically from Harvard University or commercially from Accelrys (at www (dot) accelrys (dot) corn), the Discover simulation package (included in Insight II, supra), and Dynama (available at (www dot) cs (dot) gsu (dot) edu/˜cscrwh/progs/progs (dot) html). See also an extensive list of modeling software at www (dot) netsci (dot) org/Resources/Software/Modeling/MMMD/top (dot) html.

Visual inspection and/or computational analysis of a model of a synthase or domain thereof can identify relevant features of the active site region, including, for example, one or more residues that can be mutated to alter the specificity of the synthase or domain. Similarly, visual inspection and/or computational analysis can identify candidate termini at which the synthase or domain thereof can be fused to another synthase or domain thereof to produce a functional fusion protein.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Fused Multi-Catalytic Domain Enzymes Found in Dictyostelium Discoideum Link the Catalytic Diversities of Two Complementary Polyketide Biosynthetic Systems

The following sets forth a series of experiments that demonstrate that a type III PKS domain can be fused with type I FAS/PKS domains in multi-domain enzymes. Two exemplary prototypical fusion proteins found in D. discoideum are described. These proteins include the only known covalently-tethered type III PKS enzymes.

Discovery of D. Discoideum FAS-PKS Fusion Proteins

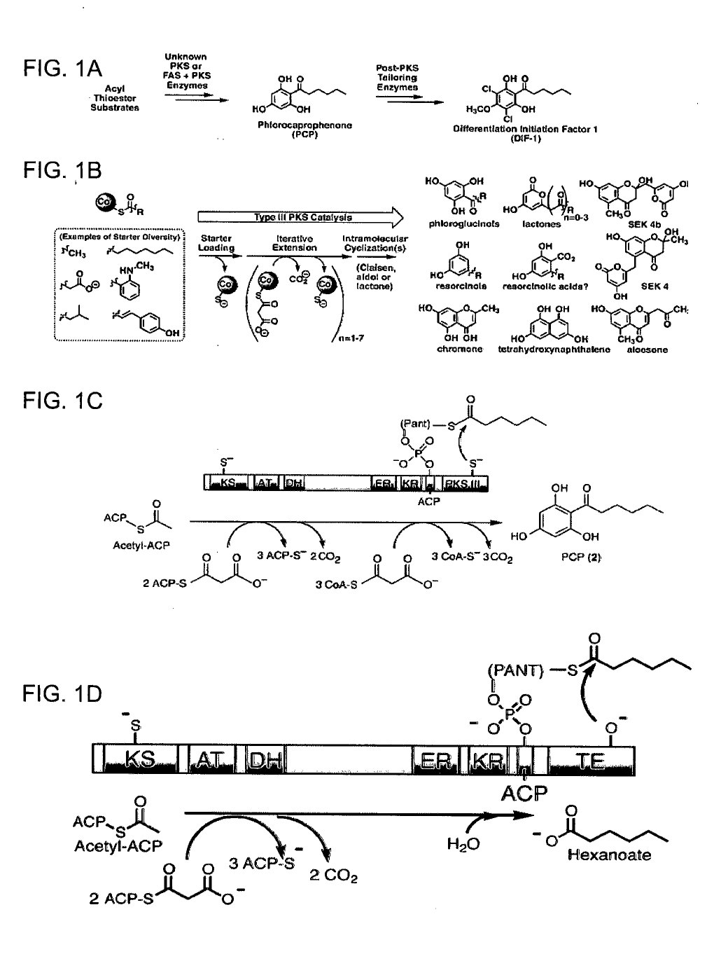

During the unusual life cycle of the model organism Dictyostelium discoideum, starvation triggers a cyclic AMP-mediated process where as many as 10⁵ undifferentiated and identical unicellular amoeba aggregate to form a multicellular slug. This “communal” slug can then migrate en masse towards light and heat[1]. Via differentiation of these identical slime mold cells into two major classes (pre-stalk and pre-spore), this mobile slug form of D. discoideum can subsequently transform itself into a vertical fruiting body. The upper mass of spore cells, awaiting germination, perches atop a stationary pedestal of vacuolated stalk cells. Differentiation Initiation Factor 1 (DIF-1) is a bioactive polyketide-derived small molecule signal that helps orchestrate this cellular differentiation in Dictyostelium[2]. Following assembly of the phlorocaprophenone (PCP) core scaffold by some previously unknown polyketide synthase activity, the DIF-1 biosynthetic pathway requires at least two more enzymatic activities to achieve the final chlorinated and O-methylated product DIF-1[3]; see FIG. 1 Panel A. However, the only DIF biosynthetic pathway enzyme previously identified is the O-methyltransferase (OMT) catalyzing the final step in the pathway[3]. Interestingly, sequence analysis reveals this slime mold S-adenosyl-L-methionine(SAM)-dependent OMT to group with OMTs from plant biosynthetic pathways, such as those acting upon phenylpropanoid lignin precursors and polyketide-derived flavonoids.

Type III polyketide synthases (PKSs) are a superfamily of structurally simple homodimeric condensing enzymes sharing homology with chalcone synthase (CHS) that typically biosynthesize phloroglucinol, resorcinol, tetrahydroxynaphthalene or 2-pyrone lactone rings from their linear polyketide intermediates[4]. These resultant multi-hydroxylated ring systems serve as the core scaffolds of thousands of biologically important natural products, including flavonoids, stilbenes, and naphthoquinones. Each type III PKS utilizes a conserved Cys-His-Asn triad within an internal active site cavity to catalyze the iterative polyketide extension, via successive condensations with, e.g., malonyl-CoA-derived acetyl units, of a starter molecule previously transferred from CoA to the enzyme's catalytic cysteine residue. Despite these conserved structural and catalytic features, type III PKS superfamily members also exhibit remarkable functional divergence, having evolved a remarkable range of catalytic specificities for starter molecule selection, number of polyketide extension steps catalyzed, and mechanism(s) of intramolecular polyketide cyclization[4] (FIG. 1 Panel B).

Although type III PKS enzymes were thought to be restricted to plants and bacteria, the resemblance of the DIF-1 polyketide precursor PCP[3] to the substituted phloriglucinol rings produced by CHS and related plant type III PKS enzymes[4] was striking. This resemblance suggested, without limitation to any particular mechanism, that a hypothetical D. discoideum CHS-like enzyme could catalyze three polyketide extensions of a thioester-activated six-carbon hexanoyl starter, followed by an intramolecular C6->C1 Claisen condensation and subsequent aromatization of this new ring to produce the phlorocaprophenone scaffold of DIF-1. As the D. discoideum genome sequencing project was underway[5], a type III PKS highly-conserved signature amino acid sequence was BLAST-searched against all possible translations of the collection of unassembled D. discoideum shotgun sequencing fragments then available in the NCBI databank. Surprisingly, this exploratory BLAST search indeed revealed raw sequencing data encoding putative proteins with significant similarity to the type III PKS signature sequence. Repeating the BLAST search using the full-length 389 amino acid sequence of alfalfa CHS returned nearly a dozen overlapping fragments whose assembly revealed two distinct sequences within the slime mold genome that aligned well with the entire alfalfa CHS query. In fact, these slime mold derived sequences are closer in amino acid identity to plant type III PKS enzymes (about 27-30%) than are most bacterial CHS-like enzymes (typically about 25% identity). And despite considerable amino acid variation between these two D. discoideum CHS-like predicted proteins (also about 30% identity), both sequences nonetheless reflect the typical type III PKS conservation of catalytic and structurally important residues throughout their lengths, suggesting they represent catalytically active and iterative polyketide synthases. However, although a few of the aligned raw sequencing fragments extended dozens of base pairs upstream of the expected start codon position, no such methionine codon was apparent for either slime mold CHS-like derived gene sequence.

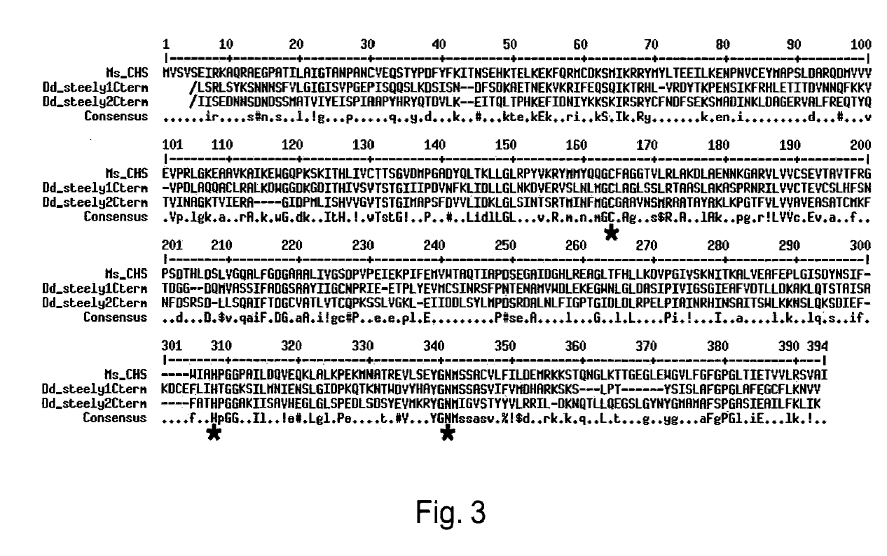

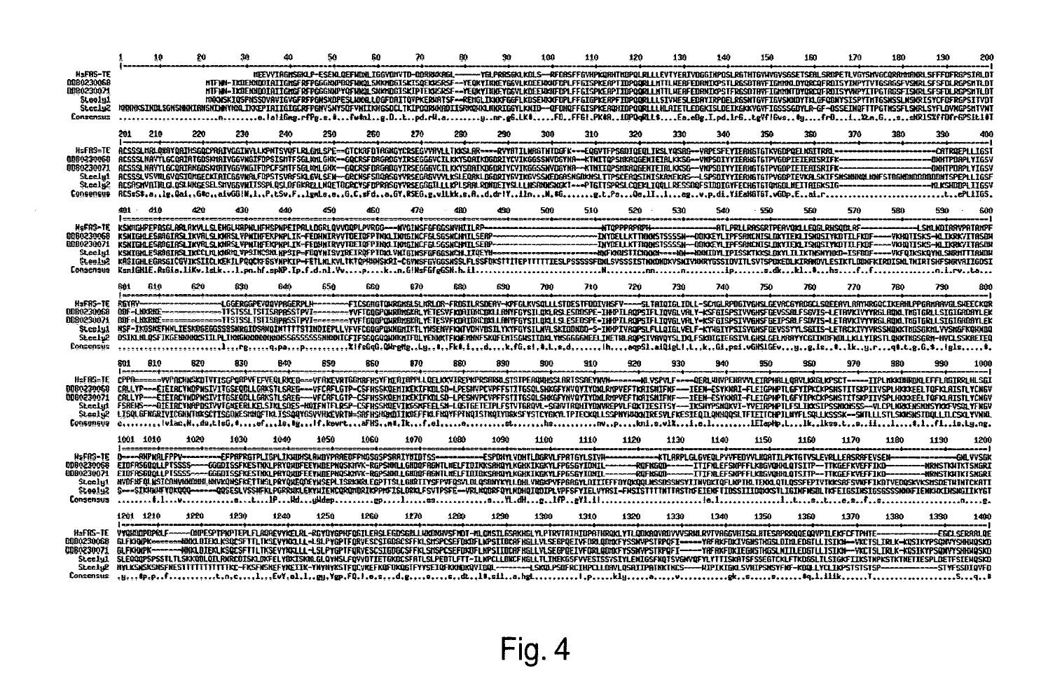

To clarify whether these putative ORFs indeed featured unprecedented N-terminal extensions relative to other type III PKS, or were instead merely inactive pseudogenes due to a lack of appropriate transcriptional and translational control elements, the collection of partially assembled D. discoideum genomic sequencing data at the Sanger Centre (http://www (dot) sanger (dot) ac (dot) uk/Projects/D_{—}discoideum/) was next searched for longer contigs containing these putative CHS-like genes. A relevant Sanger contig encompassing the upstream nucleotide environment was returned for each sequence. Both contigs were then processed for likely gene products using the ORF prediction program GeneID[6] in conjunction with a downloaded GeneID parameter file (http://www1 (dot) imim (dot) es/software/geneid/index.html#top) trained explicitly to recognize D. discoideum splice sites (i.e. introns). This GeneID analysis predicted Sanger contig_{—}9582 to contain a gene encoding a 3147 amino acid protein, with a 119 base pair intron located in the codon for residue 89, and a second intron of 73 base pairs located in the codon for residue 469. Sanger contig_{—}2219 was predicted to contain a similar gene encoding a 2968 amino acid protein with a single intron of 259 base pairs located in the codon for residue 124. The final approximately 400 residues of each of these approximately 3000 amino acid ORFs represented one of the two CHS-like sequences anticipated by the earlier BLAST results (FIG. 3). These unique Dictyostelium discoideum approximately 3000 amino acid ORFs, derived from Sanger contig_{—}9582 and contig_{—}2219, were designated “Steely1 ” and “Steely2”, respectively. The subsequently published genome sequencing project[5] annotates these Steely fusion protein ORFS as DDB0190208 (located on chromosome one) and DDB0219613 (on chromosome five), respectively.

A 700 nucleotide cDNA clone (ddv54k02) corresponding to the CHS-like C-terminus of Steely1 was found in the Japanese D. discoideum EST collection[7] (http://www (dot) csm (dot) biol (dot) tsukuba (dot) ac (dot) jp/cDNAproject (dot) html). This EST sequence, also accessible at DictyBase (http://dictybase (dot) org) as DDB0027330, confirms the physiological expression in vegetative cells of at least one of these novel Steely proteins.

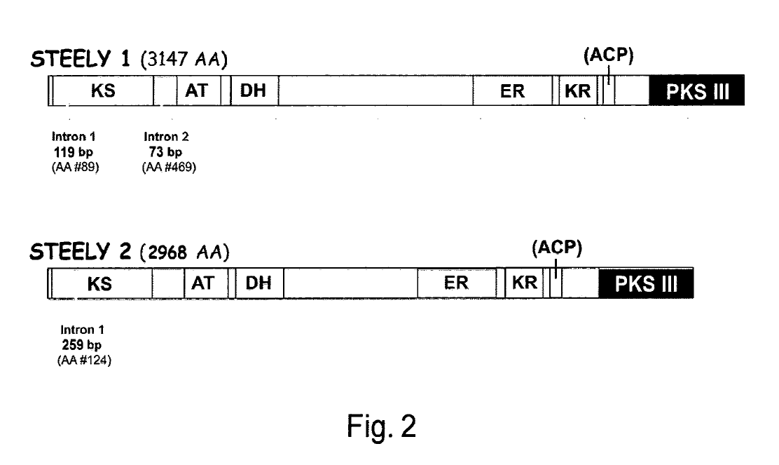

Bioinformatic analyses of the extensive N-terminal region of each putative Steely ORF predicts several enzymatic domains, whose relative order and spacing closely resembles the first six of seven covalently linked domains that constitute the type I Fatty Acid Synthase (FAS) proteins of animals and insects[8], with 30% amino acid identity with human FAS over these first approximately 2600 residues (slightly higher than the approximately 27% amino acid identity between Steely1 and Steely2). As schematically illustrated in FIG. 2, sequentially from the N-termini, these predicted Steely domains are a ketoacyl synthase (KAS I or KS), a malonyl/acyl transferase (M/AT or AT), a dehydratase (DH), an enoyl reductase (ER), a ketoreductase (KR), and a phosphopantetheine (Ppant) attachment site (which serves in type I FAS enzymes as a covalently tethered acyl carrier protein (ACP) to shuttle intermediates between the various enzymatic domains). In fatty acid biosynthesis, the M/AT domain is responsible for loading/selection of the starter moiety and malonyl-ACP extender units, while each acetyl extension of the KS-tethered starter (or intermediate) results in a carbonyl at the acyl C3 position that is subsequently reduced to a saturated methylene by the consecutive catalytic activities of the KR, DH, and ER domains. Iterative FAS chain extension and β-position saturation is terminated via simple hydrolysis of the full-length acyl thioester product by the seventh and final domain of these type I FAS proteins, a thioesterase (TE). It is this FAS C-terminal TE domain, just after the ACP-like Ppant attachment site, that is replaced by a structurally-unrelated type III PKS domain in both novel D. discoideum Steely fusion proteins described here.

In some fungi and actinomycete bacteria, repeated gene duplication and diversification of multi-domain iterative type I FAS enzymes has given rise to the predominantly non-iterative and modular type I PKS enzymes responsible for the biosynthesis of many antibiotics[9, 10]. The reaction sequence of a type I PKS module mirrors a single round of type I FAS catalysis, but typically one or more of the KR, DH, and ER domains are non-functional, resulting in diversification at the β-position (unsaturation or retention of the keto or hydroxyl moiety). Incorporation of unusual starter or extender units is another source of product diversity, as is the use of dedicated divergent copies (modules) of the multi-domain FAS enzymes for each subsequent step of polyketide chain elongation. The final module of type I PKS systems also utilize a TE domain to off-load products, sometimes via intramolecular condensation of their reactive polyketide chains to form a macrocycle. FAS-unrelated tailoring enzymes such as OMTs are also recruited into some type I PKS pathways. In many species, type I PKS modules and other pathway-associated enzymes are genomically encoded as adjacent ORFs, allowing bioinformatic analysis to provide some insights into pathway function. However, Sanger contig_{—}9582 or contig_{—}2219 contained no other such biosynthetic ORFs. An extensive D. discoideum contig (JC1c158c07.s1) containing the Sanger contig_{—}9582-derived Steely1 sequence was then located at the Dictyostelium database in Jena, Germany (http://genome (dot) imb-jena (dot) de/dictyostelium/). GeneID analysis revealed the Steely1 ORF to be the 84^{th} of 135 predicted proteins, located approximately 220 Kb from the 5′ end of this 342 Kb contig. Further bioinformatic analysis revealed no other FAS, PKS, or typical PKS-associated biosynthetic ORFs within this Steely1 -containing Jena contig. This genomic isolation of Steely1 relative to Steely2 or other enzymes of specialized metabolism suggests that the N-terminal portion of each Steely fusion protein is more likely to functionally resemble the independently-acting iterative type I FAS enzymes of primary metabolism than their functionally divergent, modular and typically clustered Type I PKS relatives.

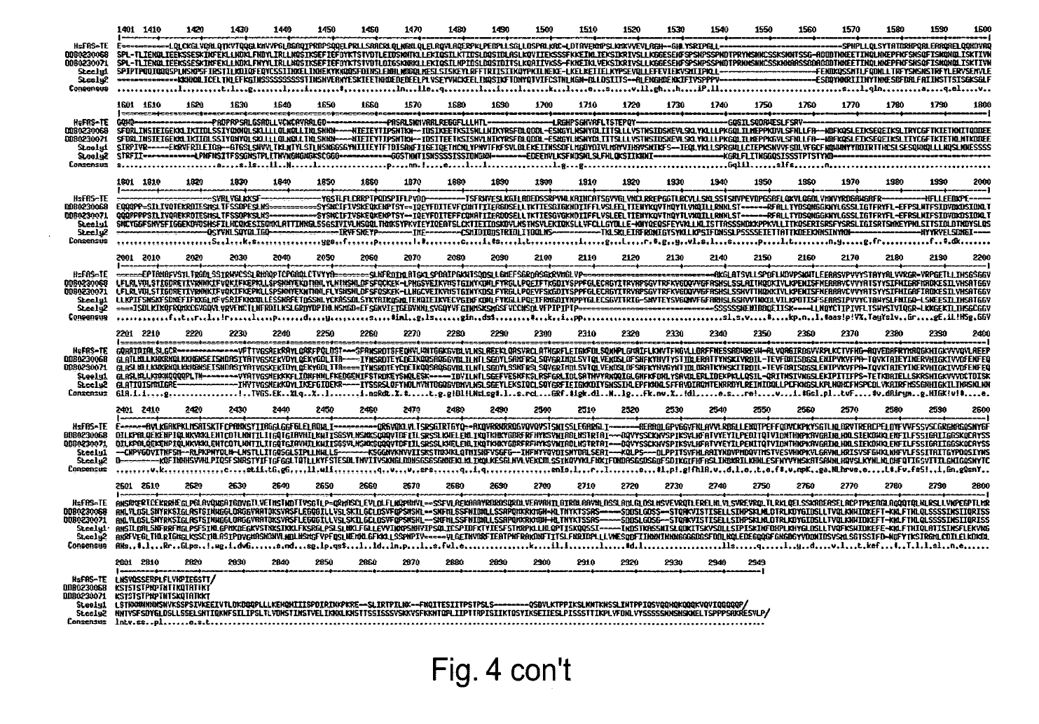

A BLAST search following completion of the D. discoideum genome project[5] revealed two D. discoideum ORFs (DDB0230068 and DDB0230071) with significant similarity to the N-terminal FAS-like portions of the two Steely proteins (FIG. 4). These additional sequences, which share 96% amino acid identity with each other, each feature stop codons following their ACP-like sixth predicted domains, and thus both approximately 2600 amino acid sequences lack any seventh domain whatsoever. While DDB023071 shares approximately 28% identity with the non-CHS like portions of both Steely proteins, DDB0230068 interestingly shares 36% amino acid identity with the non-CHS-like portion of Steely1 (DDB0190208), but less than 30% identity over aligned portions of Steely2 (DDB0219613). Although both DDB023068 and DDB023071 are annotated as FAS enzymes (solely based on sequence similarity), a bona fide type I FAS that both shares the animal FAS domain structure and lacks a C-terminal TE domain has not been reported. On the other hand, while many type I PKS modules catalyzing non-final steps of polyketide biosynthesis do share both the animal FAS-like domain structure and absence of a C-terminal TE domain (as their products are passed directly to the N-terminal KS domains of the next module), both of the TE-lacking ORFs in question are located slightly more than 100 KB from each other on chromosome two, and like the Steely genes do not appear to be surrounded by any other genes related to PKS or FAS biosynthesis. However, a few iteratively functioning non-modular type I PKS enzymes have been discovered[10], with the same active sites sometimes catalyzing different levels of reduction during different steps of polyketide chain extension [11]. Notably, at least one cloned iterative type I PKS enzyme also possesses the overall domain structure and lack of TE domain exhibited by DDB023068 and DDB023071.

In contrast to these gigantic type I FAS and type I PKS multi-domain enzymes, the multi-functional and iterative homodimeric type III PKS enzymes (found in some bacteria and all plants[4], a few fungi[12] and now at least one slime mold) appear to have evolved from the non-iterative KAS III enzymes of similarly simple architecture that prime acetyl-CoA for type II FAS biosynthesis (occurring in plants and bacteria) via a single condensation with malonyl-ACP[4]. The Steely fusion proteins' unique substitution of a type III PKS domain in place of the C-terminal TE domain required for off-loading FAS products has several important biosynthetic implications.

Firstly, molecular logic suggests that the acyl-thioester end products of the N-terminal FAS-like proteins are transferred directly from the prosthetic pantetheine arm of the ACP-like sixth domain to the catalytic cysteine residue of the type III PKS seventh domain. Although it has been previously hypothesized, based upon homology and surface residue analysis, that some bacterial type III PKS enzymes are likely to utilize ACP-tethered substrates in vivo (Austin and Noel (2003) “The chalcone synthase superfamily of type III polyketide synthases” Nat Prod Rep 20:79-110), none of these have yet been shown to prefer ACP over CoA. In the case of the covalently tethered CHS-like Steely domains, substrate channeling undoubtedly plays an important role in facilitating these type III PKS domains' proposed utilization of ACP domain-tethered substrates.

Secondly, in vivo production of an unusual saturated hexanoyl precursor, most likely catalyzed by a specialized FAS or FAS-like PKS, was a crucial prerequisite of the original hypothesis, presented above, that a hypothetical CHS-like enzyme might catalyze the final three non-reductive extensions and intramolecular Claisen cyclization of phlorocaprophenone biosynthesis. The subsequent bioinformatic discovery of two slime mold type III PKS enzymes, as well as their unprecedented covalent fusion with candidate FAS-like multi-domain proteins, reinforces and expands this initial hypothesis. These observations strongly suggest that a single Steely fusion protein can catalyze the entire biosynthesis and assembly of the 12-carbon phlorocaprophenone scaffold of DIF-1. The direct thioester transfer of a Steely N-terminal FAS product from the prosthetic Ppant moiety to the C-terminal type III PKS domain (FIG. 1 Panel C) not only eliminates the traditional requirement for a hydrolytic TE domain to off-load the FAS acyl thioester product as a free acid (FIG. 1 Panel D), but also bypasses the subsequent need for a CoA ligase to reactivate the free acid for type III PKS catalysis. It now seems evident that a single genomic event, the substitution of an iterative type III PKS domain in place of a FAS TE domain, could have in one evolutionary step conferred upon D. discoideum the ability to biosynthesize phlorocaprophenone from common primary metabolic acetyl precursors.

Engineering of Fusion Proteins

While this serendipitous fusion of type I and III domains may well have been crucial to the evolution of cell differentiation in D. discoideum, the molecular logic revealed in the novel Steely proteins' covalent fusion of a type III PKS to a multi-domain type I FAS or related PKS enzyme also has important ramifications for protein and pathway engineering of both type I and III PKS systems. Despite intense interest in type I PKS enzymes due to their production of complex bioactive natural products such as macrocycle antibiotics, the size of these multi-domain systems has thus far prevented definitive elucidation of the detailed tertiary arrangement of their active form[9, 10]. Overall assembly of FAS and PKS domains has been studied, however, and structures of various domains are available (see, e.g., Maier et al. (2006) “Architecture of mammalian fatty acid synthase at 4.5 A resolution” Science 311(5765):1258-62, Tang et al. (2006) “The 2.7-Angstrom crystal structure of a 194-kDa homodimeric fragment of the 6-deoxyerythronolide B synthase” Proc Natl Acad Sci USA. 103(30):11124-9, and discussion below). The majority of metabolic engineering of type I enzymes has involved deletion, removal, or substitution of various domains or linker regions from divergent PKS systems. In contrast, the structural simplicity and catalytic diversity that exists within the homodimeric type III PKS superfamily[4] has facilitated the atomic-resolution crystallographic comparison of several functionally divergent enzymes[13-17]. The mechanistic insights provided by subsequent mutagenic analyses and engineering successes have revealed many type III PKS design features controlling starter selection, number of polyketide extensions, and mode of intramolecular product cyclization. While the varying steric constraints imposed by residues lining the internal type III PKS active site cavity is a key determinant, in vitro analyses of these somewhat promiscuous enzymes also reveal the importance of CoA-activated starter availability in determining their range of in vivo products[4]. Although some preliminary evidence has indicated that CHS may benefit from substrate channeling in a hypothetical flavonoid pathway multi-enzyme complex[18], no conclusive proof or detailed knowledge of any biologically relevant type III PKS protein-protein interaction has yet surfaced. The presumed ability of the Steely fusion proteins to directly deliver type I FAS fatty acyl and type I PKS reduced polyketide products into a type III PKS active site, while simultaneously eliminating the diffusion-introducing need for intervening TE and CoA ligase activities to link these prolific but previously distinct biosynthetic systems, represents not only a significant evolutionary achievement by nature, but also an invaluable template for metabolic engineering of bioactive natural products. Combinatorial exploitation of the evolutionarily refined covalent linkages utilized by the D. discoideum Steely fusion proteins can significantly expand the number and diversity of polyketide products within the easy reach of in vivo metabolic engineering.

In Vitro Activities of C-Terminal PKS III Domains

Due to the large size of the full-length Steely ORFs, as well as the presence of N-terminal introns in both of their genomic sequences, initial attention was focused upon each of the Steely C-terminal type III PKS domains, the adjacent ACP-like domains, and the intervening peptide linkages that constitute the covalent fusion region. Due to the unusually high AT content throughout the D. discoideum genome[5], an unconventionally low extension temperature during PCR was used to amplify genomic DNA. Both Steely approximately 550 amino acid C-terminal di-domain constructs were cloned into a pET28-derived E. coli expression vector providing a thrombin-cleavable N-terminal poly-histidine affinity tag for purification. However, PAGE analysis of lysed cells revealed both Steely C-terminal di-domain constructs to be poorly expressed even in an E. coli strain optimized for rare codon expression (Stratagene CodonPlus). Subsequent shorter constructs representing just the C-terminal CHS-like domain of either Steely protein were also poorly expressed in E. coli, but nonetheless yielded limited amounts of relatively pure soluble protein for in vitro characterization. Proteomic analysis of co-eluting proteins revealed persistent contamination by E. coli chaperones throughout purification, suggesting that at least some portion of misfolded type III PKS domain also persisted in the soluble fraction. A synthetic gene strategy can be pursued to simultaneously optimize Steely codon usage and minimize AT content, in the expectation that the absence of D. discoideum genomic idiosyncrasies will facilitate better expression and purification of the polypeptides.

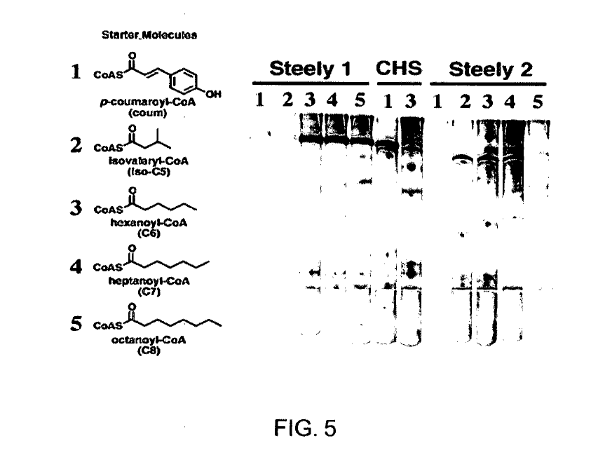

Standard in vitro assays using radiolabeled malonyl-CoA and a representative range of typical type III PKS substrates confirmed that both heterologously-expressed steely C-terminal domains catalyze iterative polyketide extension when primed with hexanoyl-CoA or other medium length aliphatic starters derived from fatty acid metabolism (FIG. 5). Neither enzyme showed significant polyketide extension activity with malonyl-CoA alone, nor when primed with acetyl-CoA or the bulky phenylpropanoid starters utilized by plant chalcone and stilbene synthases (p-coumaroyl-CoA). Interestingly, Steely2 but not Steely1 would accept isovaleryl-CoA (a short branched aliphatic) as a starter, and only Steely1 accepted a longer octanoyl-CoA starter. These differences in in vitro starter specificity are consistent with the substantial divergence of these steely active site predicted by homology modeling.

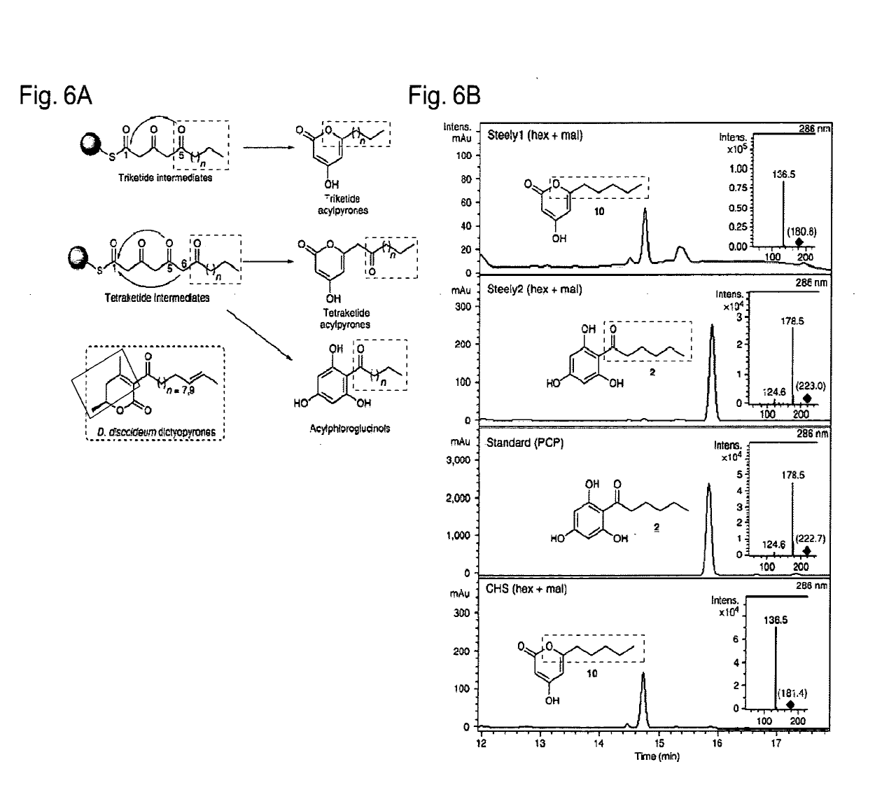

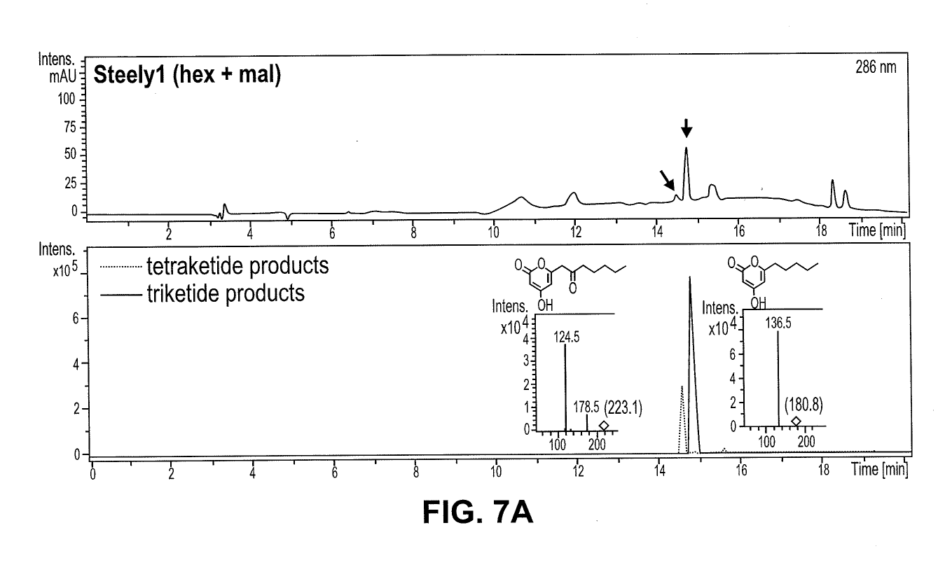

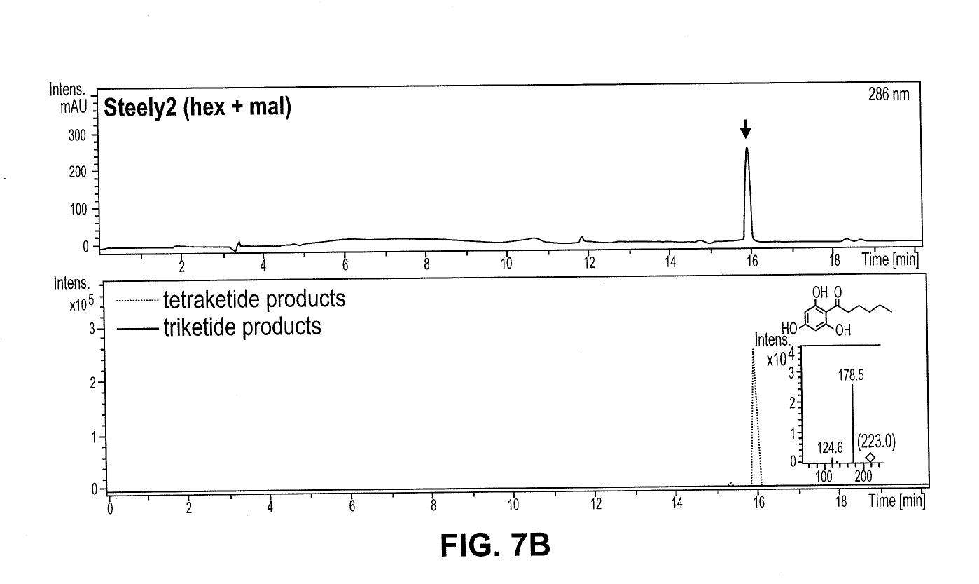

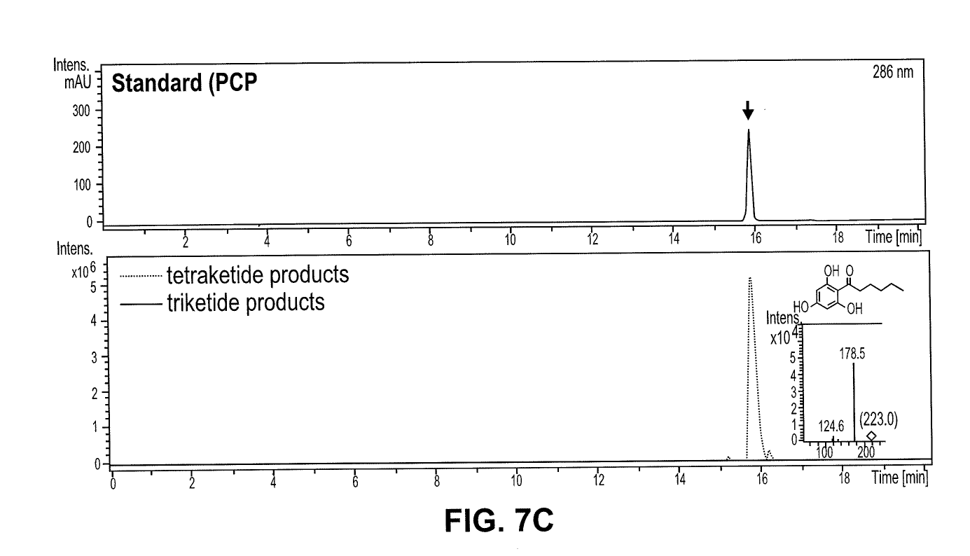

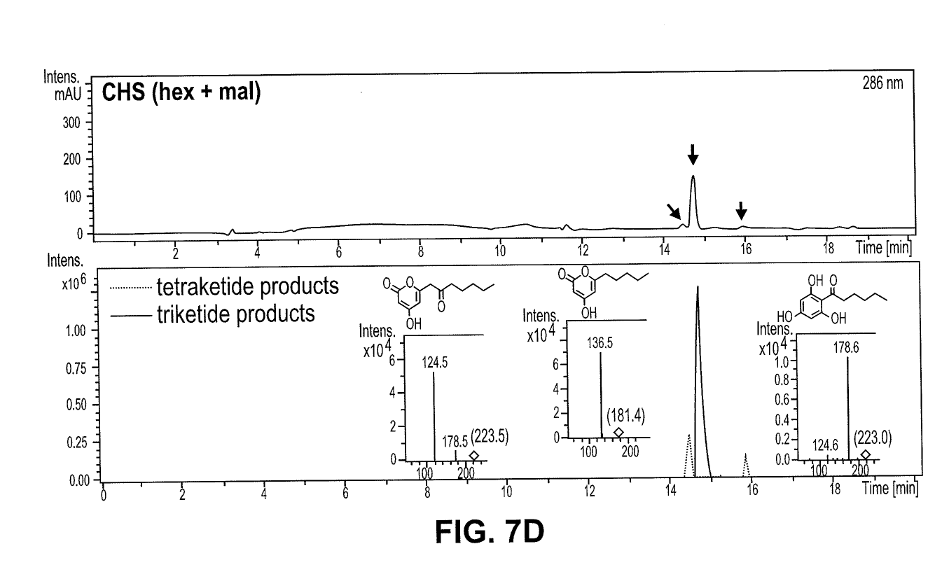

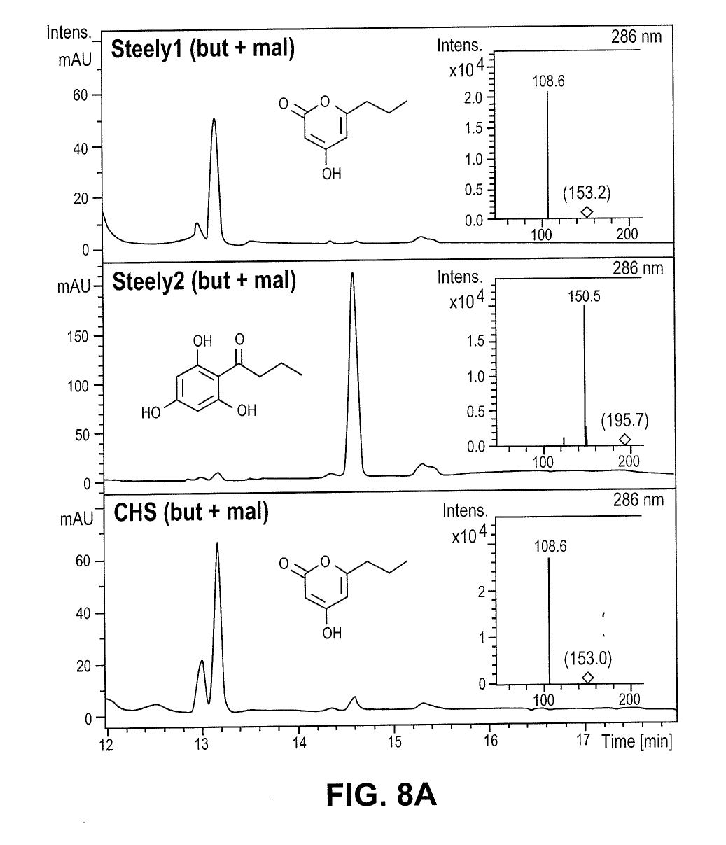

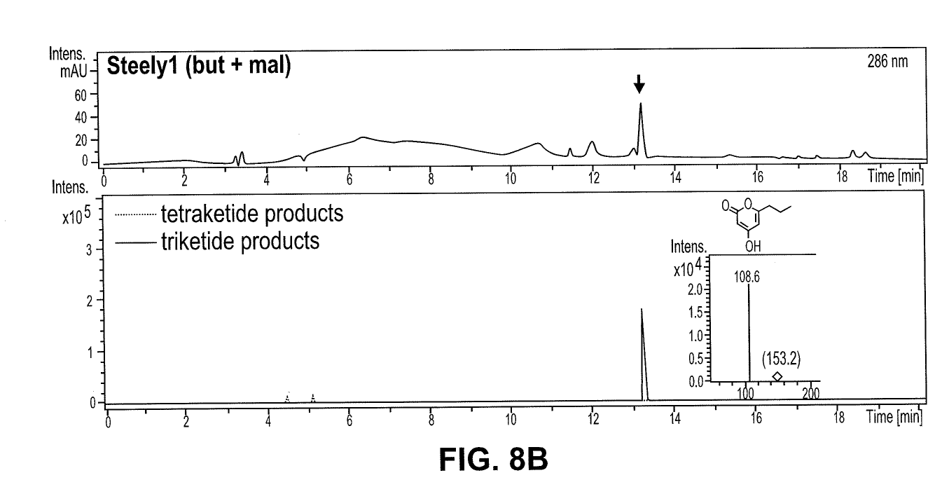

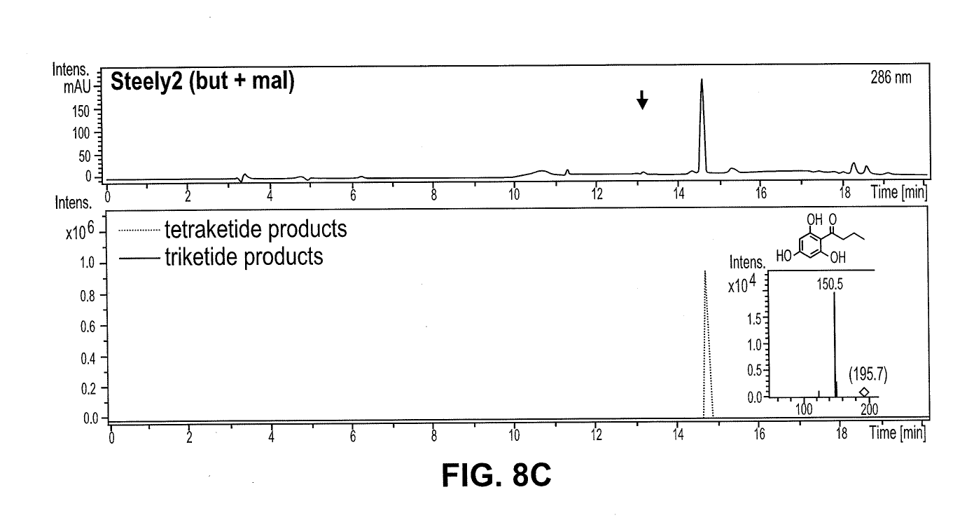

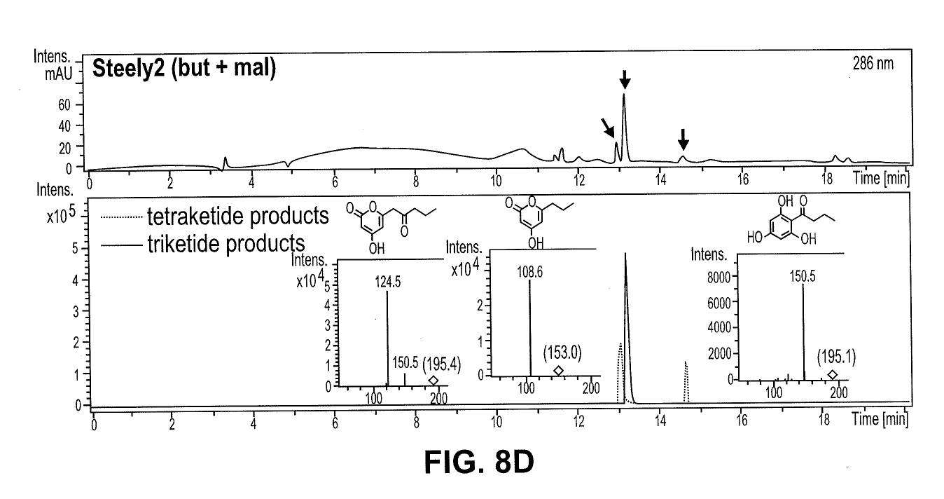

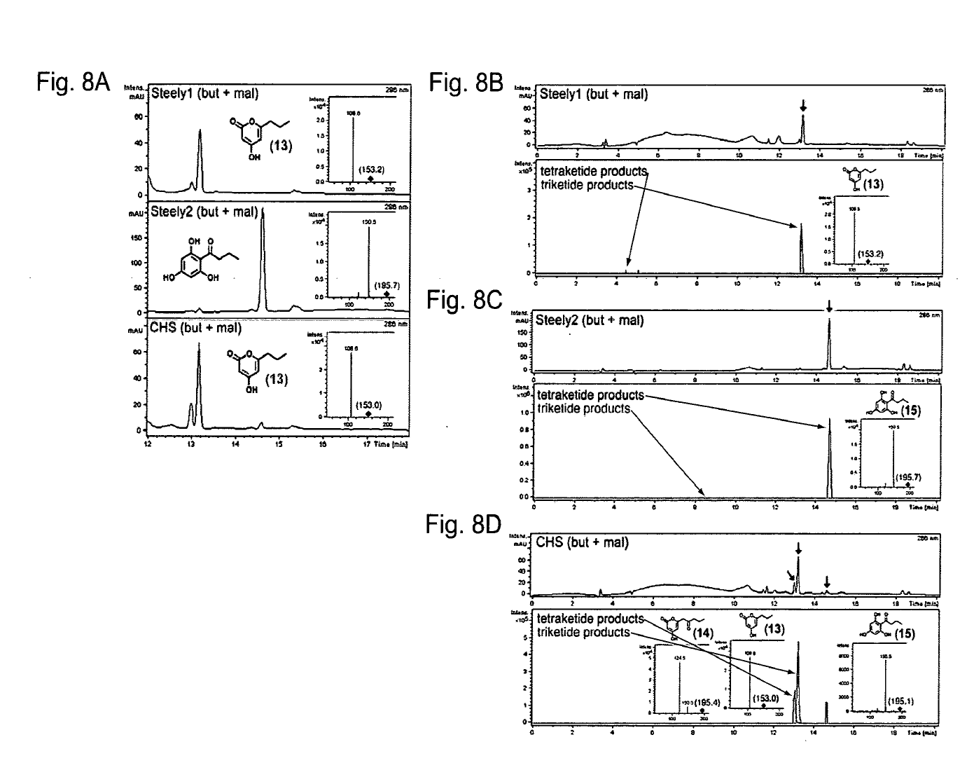

HPLC-MS-MS analyses of in vitro assays using unlabeled malonyl-CoA in conjunction with an authentic PCP standard unambiguously confirmed that the hexanoyl-primed Steely2 type III PKS domain catalyzes three rounds of polyketide chain extension and the final CHS-like intramolecular C6 to C1 Claisen condensation that is necessary to synthesize and off load the DIF-1 skeleton (FIG. 6 Panels A-B). Despite a similar preference for medium-length acyl starters (FIG. 1 Panel D), hexanoyl-primed assays of the Steely1 type III PKS domain produced only triketide (10) and tetraketide (11) lactonization-derived pyrones (FIG. 6 and FIG. 7 Panels A-D). The related D. discoideum DIF-2 acylphloroglucinol scaffold seems to be derived from a pentanoyl intermediate. Therefore, in vitro assays of each steely C-terminal domain were also primed with butanoyl-CoA (12), as pentanoyl-CoA is not commercially available. Although changing the starter moiety in this manner often alters type III PKS product cyclization[4], use of a four-carbon (rather than six-carbon) acyl starter had no effect on the cyclization fate of in vitro-generated products (13, 14, and 15) of either enzyme (FIG. 8 Panels A-D). Variation of pH and of enzyme and substrate concentrations also had no effect on the in vitro cyclization specificities reported here, although Steely1 showed reduced catalytic activity in HEPES-buffered assays. Though extracted ion chromatogram (EIC) analyses revealed trace amounts of malonyl-primed triacetic acid lactone (TAL) in CHS assays, Steely1 and Steely2 assays lacking an acyl starter (that is, either hexanoyl- or butanoyl-CoA) showed no evidence of TAL production. These assay results suggest that Steely2 can be responsible for the in vivo biosynthesis of both known acylphloroglucinol DIF scaffolds.

Structure of the Steely1 C-Terminal Type III PKS Domain

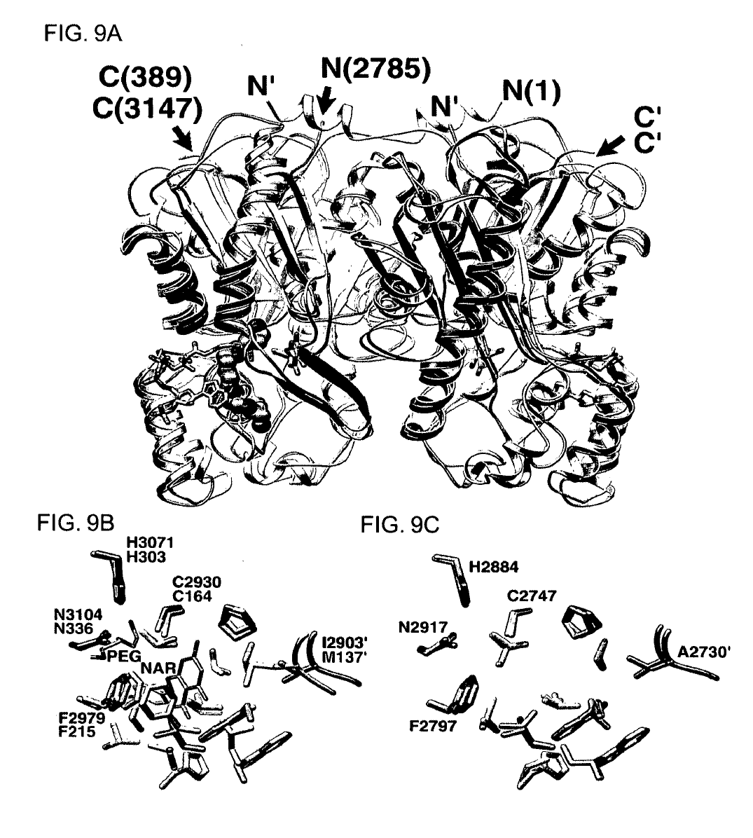

A single batch of diffraction-quality crystals of the heterologously-expressed CHS-like C-terminal domain of Steely1 was produced. A resulting 2.9 Angstrom resolution data set was solved by molecular replacement using Phaser and two copies of a monomeric homology model derived from the alfalfa CHS crystal structure; see FIG. 9 Panels A-C and Table 2. Comparison of the crystallographically refined Steely1 model to previous crystal structures reveals conservation of the internal active site cavity, the Cys-His-Asn catalytic triad, and the overall type III PKS tertiary structure, despite minor conformational differences in the protein backbone over a few contiguous sections of the first 60 or so residues. Without intending to be limited to any particular mechanism, the loose packing of a few elements of secondary structure seems to suggest the possibility of additional but quite narrow entrances into the active site cavity, conceivably relevant in the context of the entire Steely multi-domain complex. However, this ambiguous hint in the low-resolution crystal structure may just reflect the decreased stability of the heterologously expressed Steely1 C-terminal domain encoded by the truncated D. discoideum gene. Additional electron density present in the traditional pantetheine-binding entrance is consistent with a bound molecule of the PEG precipitant introduced during crystallization. Additional description of the structure after an additional round of refinement can be found in Austin et al. (2006) “Biosynthesis of Dictyostelium discoideum differentiation-inducing factor by a hybrid type I fatty acid-type III polyketide synthase” Nature Chemical Biology 2:494-502.

TABLE 2
Steely1 crystallographic and refinement statistics.

| | Steely1 C-terminal domain |
|---|---|
| Space group | P2(1)2(1)2(1) |
| Unit cell dimensions (Å, °) | a = 82.0 |
| | b = 83.3 |
| | c = 114.3 |
| | α = β = γ = 90 |
| Wavelength (Å) | 0.980 |
| Resolution (Å) | 2.9 |
| Total reflections | 75,933 |
| Unique reflections | 17,517 |
| Completeness^{a} (%) | 99.6 (99.7) |
| I/σ^{a} | 12.1 (4.4) |
| R_{sym}^{a,b} | 22.2 (53.5) |
| R_{cryst}^{c}/R_{free}^{d} (%) | 20.0/23.2 |
| Protein atoms | 5583 |
| Ligand atoms | 19 |
| Water molecules | 366 |
| R.m.s.d. bond lengths (Å) | 0.020 |
| R.m.s.d. bond angles (deg) | 1.9 |
| Average B-factor - protein (Å²) | 22.1 |
| Average B-factor - solvent (Å²) | 22.2 |

^{a}Number in parenthesis is for the highest resolution shell;
^{b}R_{sym} = Σ|Iₕ − <Iₕ>|/ΣIₕ, where <Iₕ> is the average intensity over symmetry equivalent reflections;
^{c}R-factor = Σ|F_{obs} − F_{calc}|/ΣF_{obs}, where summation is over the data used for refinement;
^{d}R_{free}-factor is the same definition as for R-factor, but includes only 5% of data excluded from refinement.

Notably, this new crystal structure also revealed the same homodimeric domain assembly common to all other structurally characterized CHS-like enzymes[13-17]. Twin copies of the multi-domain polypeptides encoded by type I PKS modules, as well as the higher eukaryotic type I FAS systems discussed here, form binary complexes due to homodimeric interactions of some, but not all, of their domains and linker regions[8-10, 22]. While some evidence suggested that type I FAS proteins might utilize a monomeric quaternary form of TE, due to a hypothesized antiparallel homodimeric assembly of their multi-domain proteins[22], more recent studies support an alternative model that includes homodimeric assemblies of both KS and TE domains[8]. Even more recent studies show overall parallel assembly mediated by dimerization of KS, DH, and ER domains; these studies also support FAS monomeric TE domains (Maier et al. (2006) “Architecture of mammalian fatty acid synthase at 4.5 A resolution” Science 311(5765):1258-62). It is definitively established, however, that the more functionally diverse but evolutionarily related (by their common αβ-hydrolase fold) TE domains of type I PKS enzymes indeed function as homodimers[10, 23]. A recent study shows the same dimerization architecture for a KS+AT didomain fragment of a modular type I PKS as observed above for mammalian FAS (Tang et al. (2006) “The 2.7-Angstrom crystal structure of a 194-kDa homodimeric fragment of the 6-deoxyerythronolide B synthase” Proc Natl Acad Sci USA. 103(30):11124-9).

Interestingly, as noted above, FAS C-terminal TE domains are believed not to homodimerize in the physiological and catalytically active form of the FAS complex. Conversely, type I PKS C-terminal TE domains definitely do form tight homodimers in their active complexes, suggesting the quaternary association of the Steely proteins is more likely to resemble type I PKS enzyme complexes, rather than those of type I FAS enzymes. Another interesting perspective is also suggested by comparison of the Steely fusion regions to modular PKS domains. While FAS and PKS TE domains all possess the αβ-hydrolase protein fold, all β-keto condensing enzymes possess a common αβαβα fold. Just as the confirmation of polyketide extension catalysis in heterologously-expressed Steely C-terminal domains described herein implies they do not act simply as surrogate thioesterase domains, the protein fold relationship of type III PKS enzymes to the KS domains of modular type I PKS domains also suggests the best quaternary model for the Steely fusion domain association may actually be the interaction between the C-terminal ACP domain of one type I PKS module and the N-terminal KS domain of the covalently linked downstream type I PKS module, as illustrated by the domain organization and interactions of the well-studied DEBS proteins involved in erythromycin biosynthesis.

Thus the homodimeric Steely type III PKS domains appear quite capable of facile TE-like interactions with their adjacent ACP domains, given some evolutionary fine-tuning of their covalent peptide linkages. An additional perspective into the suitability of CHS-like enzymes for interaction with type I ACP domains lies in the conserved αβαβα- or thiolase-fold of all FAS and PKS condensing enzymes. The C-terminal ACP domains of type I PKS modules that do not contain a reaction-terminating TE domain instead directly hand off their intermediate polyketide products to the N-terminal KS domain of the next module, in a cross-module interaction known to be linker-dependent. This known interaction of modular PKSs seems quite analogous to the proposed one-way transfer of Steely N-terminal intermediates from their ACP domain pantetheine arm to the catalytic cysteines of their CHS-like domains.

The Steely proteins constitute a novel and genuine fusion of the complimentary catalytic abilities of two powerfully diverse but heretofore separate biosynthetic systems. Single copies of roughly 400 amino acid iterative and multi-functional type III PKS enzymes, when incorporated as C-terminal domains, can produce TE-like hydrolytic or cyclization-mediated product off-loading, while also functionally replacing multiple PKS modules of 1000-3000 amino acids each. Newly discovered CHS-like enzymes with specificities for longer starters[17], more polyketide extension steps[24], or novel product cyclizations[25] continue to expand the previously known range[4] of type III PKS catalysis. And given the known and potential genetic and functional diversity of modular and iterative type I PKS systems[9-11], the novel domain structure of the D. discoideum Steely proteins described here reveal an untapped but evolutionarily-refined template for the combinatorial construction of a plethora of novel fusion enzymes for metabolic and pathway engineering.

Additional details and discussion of the Steely1 and Steely2 fusion proteins can be found in Austin et al. (2006) “Biosynthesis of Dictyostelium discoideum differentiation-inducing factor by a hybrid type I fatty acid-type III polyketide synthase” Nature Chemical Biology 2:494-502, which is hereby incorporated by reference. Steely1 is DDB0190208 at dictyBase (dictybase (dot) org) and Steely2 is DDB0219613. The atomic coordinates and structure factors of the Steely1 type III PKS domain crystal structure have been deposited in the Protein Data Bank (PDB) under the accession code 2H84.

### Experimental Procedures

Cloning, Expression and Purification

Three C-terminal constructs of varying length were designed for each D. discoideum Steely fusion protein. Each sequence was amplified from genomic DNA (a gift from S. Merlot and R. Firtel) using complimentary oligonucleotides with restriction sites for direct cloning into the pHIS-8 expression vector, as previously described[26]. Each construct was confirmed by automated nucleotide sequencing (Salk Institute DNA sequencing facility). Following overexpression in E. coli BL21(DE3) or CodonPlus (Stratagene) cells, recombinant proteins were purified to near-homogeneity (with persistent contamination by E. coli chaperone proteins, as confirmed by N-terminal sequencing of PAGE protein bands), concentrated to between 0.5 and 15 mg/ml, and stored at −80° C., following buffer exchange into 12 mM HEPES (pH 7.5), 25 mM NaCl, and 5 mM DTT, as described previously[26].

Enzyme Assays

Standard 100 μL in vitro assays of heterologously expressed Steely C-terminal domains using [14-C]malonyl-CoA and various CoA-linked starters were conducted, extracted with ethyl acetate, analyzed by reverse-phase TLC, and visualized by autoradiography as previously reported[15].

For HPLC-MS-MS analyses 25 μl injections of similarly prepared overnight reactions (but without organic extraction) buffered with 100 mM Bis-Tris Propane (pH 7.0), using unlabeled malonyl-CoA, were used. LC-MS-MS analyses were carried out on an Agilent 1100 HPLC with an integrated Agilent LC/MSD Trap XCT ion trap mass spectrometer, using a reversed-phase C18 column (4.6×150 mm; Gemini) maintained at 30° C. A gradient mobile phase ramped from 5% to 100% acetonitrile in water (with each solvent containing 0.1% v/v formic acid) between minutes 3 and 13 of a 25-min run using a flow rate of 0.5 ml min⁻¹ and a 0.1 ml min⁻¹ post column injection of 20 mM ammonium acetate in water. UV absorbance was monitored at 286 nm.

PCP was identified by direct HPLC-MS-MS comparison with an authentic synthetic standard, kindly provided by S. Horinouchi and N. Funa. Other hexanoyl- and butanoyl-primed enzymatic products were identified by comparing their relative HPLC elution times and negative MS-MS fragmentation patterns with previously published LC-MS-MS analyses of authentic standards (Funa et al. (2002) “Properties and substrate specificity of RppA, a chalcone synthase-related polyketide synthase in Streptomyces griseus” J Biol Chem 277:4628-4635). EICs with parent ion masses of plausible polyketide products were used to detect trace amounts of minor enzymatic products, but only triketide and tetraketide products were observed.

Characterization of hexanoyl-derived products: triketide acylpyrone (4-hydroxy-6-pentyl-pyran-2-one), LC retention time 14.7 min, negative MS181.4 [M-H]⁻, negative MS-MS (precusor ion at m/z 181.4) 136.5 [M-H-CO₂]⁻; tetraketide acylpyrone (4-hydroxy-6-(2-oxo-heptyl)-pyran-2-one), LC retention time 14.5 min, negative MS 223.5 [M-H]⁻, negative MS-MS (precusor ion at m/z 223.5) major 124.5 [C₆H₅O₃]⁻ and minor 178.5 [M-H-CO₂]⁻; tetraketide acylphloroglucinol (1-(2,4,6-trihydroxyphenyl)-hexan-1-one, PCP), LC retention time 15.9 min, negative MS 222.7 [M-H]⁻, negative MS-MS (precusor ion at m/z 222.7) major 178.5 [M-H-44]⁻ and minor 124.6 [C₆H₅O₃]⁻.

Butanoyl-derived products determined by reverse phase HPLC-MS-MS analysis are as follows: triketide acyl pyrone (=4-hydroxy-6-propyl-pyran-2-one): LC retention time=13.2 min., negative MS 153.6 [M-H]⁻, negative MSMS (precursor ion at m/z 153.6) 108.5 [M-H-CO₂]⁻. tetraketide acyl pyrone (=4-hydroxy-6-(2-oxo-pentyl)-pyran-2-one): LC retention time=13.0 min.; negative MS 195.4 [M-H]⁻; negative MSMS (precursor ion at m/z 195.4) major 124.5 [C₆H₅O₃]⁻, minor 150.5 [M-H-CO₂]⁻. tetraketide acyl phloroglucinol (=1-(2,4,6-trihydroxy-phenyl)-butan-1-one): LC retention time=14.6 min.; negative MS 195.7 [M-H]⁻; negative MSMS (precursor ion at m/z 195.7) major 150.5 [M-H-44]⁻, minor 124.6 [C₆H₅O₃]⁻.

Crystallization and Data Collection

Crystals of the heterologously expressed Steely1 medium length (S1M) construct were obtained by vapor diffusion in hanging drops consisting of a 1:1 mixture of protein and crystallization buffer. The crystallization buffer contained 17% (w/v) PEG 17500, 0.5 M ammonium formate, and 100 mM MOPSO⁻Na⁺ buffer at pH 7.0. Prior to freezing in liquid nitrogen, S1M crystals were passed through a cryogenic buffer identical to the crystallization buffer except for the use of 19% (w/v) PEG 17500 and the inclusion of 18% (v/v) glycerol.

The D. discoideum C-terminal S1M construct crystallized in the P2₁2₁2₁ space group, with unit cell dimensions of a=82.0 Å, b=83.3 Å, c=114.3 Å, α=β=γ=90°, with two monomers (one physiological homodimer) in the asymmetric unit.

Data were collected at the European Synchrotron Radiation Facility (ESRF). Indexation and integration of diffraction images, as well as scaling and merging of reflections, was achieved using the HKL suite [27], and data reduction was completed with CCP4 programs[28].

Structure Determination and Refinement

The S1M crystal structure was solved by molecular replacement using PHASER[29], and two copies of a monomeric MODELLER[30]-generated homology model based upon the alfalfa CHS2 crystal structure[13].

Solutions were iteratively refined using CNS[31]. Inspection of the |2F_{O}-F_{c}| and |F_{O}-F_{c}| electron density maps and model building were performed in O[32]. Current refinement statistics are listed in Table 1. Each residue's backbone conformation was categorized (by CCP4's PROCHECK analysis of Ramachandran plots[28]) as either core (most favorable), allowed, generally allowed, or disallowed. The percentage of refined Steely1 C-terminal domain residues in each group is 87.6%, 11.3%, 0.8%, and 0.3%, respectively. Disallowed residues are those involved in a hairpin turn at the protein surface (distant from the active site). Notably, similar disallowed backbone conformations were observed in other type III PKS crystal structures[4, 13, 15, 33].

### Steely 1 and 2 Sequences

TABLE 3
Steely1 and Steely2 amino acid and polynucleotide sequences

SEQ ID NO: 1, Steely1 amino acid sequence, 3147 aa

| | |
|---|---|
| 1 | MNKNSKIQSP NSSDVAVIGV GFRFPGNSND PESLWNNLLD GFDAITQVPK ERWATSFREM |
| 61 | GLIKNKFGGF LKDSEWKNFD PLFFGIGPKE APFIDPQQRL LLSIVWESLE DAYIRPDELR |
| 121 | GSNTGVFIGV SNNDYTKLGF QDNYSISPYT MTGSNSSLNS NRISYCFDFR GPSITVDTAC |
| 181 | SSSLVSVNLG VQSIQMGECK IAICGGVNAL FDPSTSVAFS KLGVLSENGR CNSFSDQASG |
| 241 | YVRSEGAGVV VLKSLEQAKL DGDRIYGVIK GVSSNEDGAS NGDKNSLTTP SCEAQSINIS |
| 301 | KAMEKASLSP SDIYYIEAHG TGTPVGDPIE VKALSKIFSN SNNNQLNNFS TDGNDNDDDD |
| 361 | DDNTSPEPLL IGSFKSNIGH LESAAGIASL IKCCLMLKNR MLVPSINCSN LNPSIPFDQY |
| 421 | NISVIREIRQ FPTDKLVNIG INSFGFGGSN CHLIIQEYNN NFKNNSTICN NNNNNNNNID |
| 481 | YLIPISSKTK KSLDKYLILI KTNSNYHKDI SFDDFVKFQI KSKQYNLSNR MTTIANDWNS |
| 541 | FIKGSNEFHN LIESKDGEGG SSSSNRGIDS ANQINTTTTS TINDIEPLLV FVFCGQGPQW |
| 601 | NGMIKTLYNS ENVFKNTVDH VDSILYKYFG YSILNVLSKI DDNDDSINHP IVAQPSLFLL |
| 661 | QIGLVELFKY WGIYPSISVG HSFGEVSSYY LSGIISLETA CKIVYVRSSN QNKTMGSGKM |
| 721 | LVVSMGFKQW NDQFSAEWSD IEIACYNAPD SIVVTGNEER LKELSIKLSD ESNQIFNTFL |
| 781 | RSPCSFHSSH QEVIKGSMFE ELSNLQSTGE TEIPLFSTVT GRQVLSGHVT AQHIYDNVRE |
| 841 | PVLFQKTIES ITSYIKSHYP SNQKVIYVEI APHPTLFSLI KKSIPSSNKN SSSVLCPLNR |
| 901 | KENSNNSYKK FVSQLYFNGV NVDFNFQLNS ICDNVNNDHH LNNVKQNSFK ETTNSLPRYQ |
| 961 | WEQDEYWSEP LISRKNRLEG PTTSLLGHRI IYSFPVFQSV LDLQSDNYKY LLDHLVNGKP |
| 1021 | VFPGAGYLDI IIEFFDYQKQ QLNSSDSSNS YIINVDKIQF LNPIHLTENK LQTLQSSFEP |
| 1081 | IVTKKSAFSV NFFIKDTVED QSKVKSMSDE TWTNTCKATI SLEQQQPSPS STLTLSKKQD |
| 1141 | LQILRNRCDI SKLDKFELYD KISKNLGLQY NSLFQVVDTI ETGKDCSFAT LSLPEDTLFT |
| 1201 | TILNPCLLDN CFHGLLTLIN EKGSFVVESI SSVSIYLENI GSFNQTSVGN VQFYLYTTIS |
| 1261 | KATSFSSEGT CKLFTKDGSL ILSIGKFIIK STNPKSTKTN ETIESPLDET FSIEWQSKDS |
| 1321 | PIPTPQQIQQ QSPLNSNPSF IRSTILKDIQ FEQYCSSIIH KELINHEKYK NQQSFDINSL |
| 1381 | ENHLNDDQLM ESLSISKEYL RFFTRIISII KQYPKILNEK ELKELKEIIE LKYPSEVQLL |
| 1441 | EFEVIEKVSM IIPKLLFEND KQSSMTLFQD NLLTRFYSNS NSTRFYLERV SEMVLESIRP |
| 1501 | IVREKRVFRI LEIGAGTGSL SNVVLTKLNT YLSTLNSNGG SGYNIIIEYT FTDISANFII |
| 1561 | GEIQETMCNL YPNVTFKFSV LDLEKEIINS SDFLMGDYDI VLMAYVIHAV SNIKFSIEQL |
| 1621 | YKLLSPRGWL LCIEPKSNVV FSDLVFGCFN QWWNYYDDIR TTHCSLSESQ WNQLLLNQSL |
| 1681 | NNESSSSSNC YGGFSNVSFI GGEKDVDSHS FILHCQKESI SQMKLATTIN NGLSSGSIVI |
| 1741 | VLNSQQLTNM KSYPKVIEYI QEATSLCKTI EIIDSKDVLN STNSVLEKIQ KSLLVFCLLG |
| 1801 | YDLLENNYQE QSFEYVKLLN LISTTASSSN DKKPPKVLLI TKQSERISRS FYSRSLIGIS |
| 1861 | RTSMNEYPNL SITSIDLDTN DYSLQSLLKP IFSNSKFSDN EFIFKKGLMF VSRIFKNKQL |
| 1921 | LESSNAFETD SSNLYCKASS DLSYKYAIKQ SMLTENQIEI KVECVGINFK DNLFYKGLLP |
| 1981 | QEIFRMGDIY NPPYGLECSG VITRIGSNVT EYSVGQNVFG FARHSLGSHV VTNKDLVILK |
| 2041 | PDTISFSEAA SIPVVYCTAW YSLFNIGQLS NEESILIHSA TGGVGLASLN LLKMKNQQQQ |
| 2101 | PLTNVYATVG SNEKKKFLID NFNNLFKEDG ENIFSTRDKE YSNQLESKID VILNTLSGEF |
| 2161 | VESNFKSLRS FGRLIDLSAT HVYANQQIGL GNFKFDHLYS AVDLERLIDE KPKLLQSILQ |
| 2221 | RITNSIVNGS LEKIPITIFP STETKDAIEL LSKRSHIGKV VVDCTDISKC NPVGDVITNF |
| 2281 | SMRLPKPNYQ LNLNSTLLIT GQSGLSIPLL NWLLSKSGGN VKNVVIISKS TMKWKLQTMI |
| 2341 | SHFVSGFGIH FNYVQVDISN YDALSEAIKQ LPSDLPPITS VFHLAAIYND VPMDQVTMST |
| 2401 | VESVHNPKVL GAVNLHRISV SFGWKLNHFV LFSSITAITG YPDQSIYNSA NSILDALSNF |
| 2461 | RRFMGLPSFS INLGPMKDEG KVSTNKSIKK LFKSRGLPSL SLNKLFGLLE VVINNPSNHV |
| 2521 | IPSQLICSPI DFKTYIESFS TMRPKLLHLQ PTISKQQSSI INDSTKASSN ISLQDKITSK |
| 2581 | VSDLLSIPIS KINFDHPLKH YGLDSLLTVQ FKSWIDKEFE KNLFTHIQLA TISINSFLEK |
| 2641 | VNGLSTNNNN NNNSNVKSSP SIVKEEIVTL DKDQQPLLLK EHQHIIISPD IRINKPKRES |
| 2701 | LIRTPILNKF NQITESIITP STPSLSQSDV LKTPPIKSLN NTKNSSLINT PPIQSVQQHQ |
| 2761 | KQQQKVQVIQ QQQQPLSRLS YKSNNNSFVL GIGISVPGEP ISQQSLKDSI SNDFSDKAET |
| 2821 | NEKVKRIFEQ SQIKTRHLVR DYTKPENSIK FRHLETITDV NNQFKKVVPD LAQQACLRAL |
| 2881 | KDWGGDKGDI THIVSVTSTG IIIPDVNFKL IDLLGLNKDV ERVSLNLMGC LAGLSSLRTA |
| 2941 | ASLAKASPRN RILVVCTEVC SLHFSNTDGG DQMVASSIFA DGSAAYIIGC NPRIEETPLY |
| 3001 | EVMCSINRSF PNTENAMVWD LEKEGWNLGL DASIPIVIGS GIEAFVDTLL DKAKLQTSTA |
| 3061 | ISAKDCEFLI HTGGKSILMN IENSLGIDPK QTKNTWDVYH AYGNMSSASV IFVMDHARKS |
| 3121 | KSLPTYSISL AFGPGLAFEG CFLKNVV |

SEQ ID NO: 2, Steely2 amino acid sequence, 2968 aa

| | |
|---|---|
| 1 | MNNNKSINDL SGNSNNNIAN SNINNYNNLI KKEPIAIIGI GCRFPGNVSN YSDFVNIIKN |
| 61 | GSDCLTKIPD DRWNADIISR KQWKLNNRIG GYLKNIDQFD NQFFGISPKE AQHIDPQQRL |
| 121 | LLHLAIETLE DGKISLDEIK GKKVGVFIGS SSGDYLRGFD SSEINQFTTP GTNSSFLSNR |
| 181 | LSYFLDVNGP SMTVNTACSA SMVAIHLGLQ SLWNGESELS MVGGVNIISS PLQSLDFGKA |
| 241 | GLLNQETDGR CYSFDPRASG YVRSEGGGIL LLKPLSAALR DNDEIYSLLL NSANNSNGKT |
| 301 | PTGITSPRSL CQEKLIQQLL RESSDQFSID DIGYFECHGT GTQMGDLNEI TAIGKSIGML |
| 361 | KSHDDPLIIG SVKASIGHLE GASGICGVIK SIICLKEKIL PQQCKFSSYN PKIPFETLNL |
| 421 | KVLTKTQPWN NSKRICGVNS FGVGGSNSSL FLSSFDKSTT ITEPTTTTTI ESLPSSSSSF |
| 481 | DNLSVSSSIS TNNDNDKVSN IVNNRYGSSI DVITLSVTSP DKEDLKIRAN DVLESIKTLD |
| 541 | DNFKIRDISN LTNIRTSHFS NRVAIIGDSI DSIKLNLQSF IKGENNNNKS IILPLINNGN |
| 601 | NNNNNNNNSS GSSSSSSNNN NICFIFSGQG QQWNKMIFDL YENNKTFKNE MNNFSKQFEM |
| 661 | ISGWSIIDKL YNSGGGGNEE LINETWLAQP SIVAVQYSLI KLFSKDIGIE GSIVLGHSLG |
| 721 | ELMAAYYCGI INDFNDLLKL LYIRSTLQNK TNGSGRMHVC LSSKAEIEQL ISQLGFNGRI |
| 781 | VICGNNTMKS CTISGDNESM NQFTKLISSQ QYGSVVHKEV RTNSAFHSHQ MDIIKDEFFK |
| 841 | LFNQYFPTNQ ISTNQIYDGK SFYSTCYGKY LTPIECKQLL SSPNYWWKNI RESVLFKESI |
| 901 | EQILQNHQQS LTFIEITCHP ILNYFLSQLL KSSSKSNTLL LSTLSKNSNS IDQLLILCSK |
| 961 | LYVNNLSSIK WNWFYDKQQQ QQSESLVSSN FKLPGRRWKL EKYWIENCQR QMDRIKPPMF |
| 1021 | ISLDRKLFSV TPSFEVRLNQ DRFQYLNDHQ IQDIPLVPFS FYIELVYASI FNSISTTTTN |
| 1081 | TTASTMFEIE NFTIDSSIII DQKKSTLIGI NFNSDLTKFE IGSINSIGSG SSSNNNFIEN |
| 1141 | KWKIHSNGII KYGTNYLKSN SKSNSFNEST TTTTTTTTTT KCFKSFNSNE FYNEIIKYNY |
| 1201 | NYKSTFQCVK EFKQFDKQGT FYYSEIQFKK NDKQVIDQLL SKQLPSDFRC IHPCLLDAVL |
| 1261 | QSAIIPATNK TNCSWIPIKI GKLSVNIPSN SYFNFKDQLL YCLIKPSTST STSPSTYFSS |
| 1321 | DIQVFDKKNN NLICELTNLE FKGINSSSSS SSSSSTINSN VEANYESKIE ETNHDEDEDE |
| 1381 | ELPLVSEYVW CKEELINQSI KFTDNYQTVI FCSTNLNGND LLDSIITSAL ENGHDENKIF |
| 1441 | IVSPPPVESD QYNNRIIINY TNNESDFDAL FAIINSTTSI SGKSGLFSTR FIILPNFNSI |
| 1501 | TFSSGNSTPL ITNVNGNGNG KSCGGGGGST NNTISNSSSS ISSIDNGNNE DEEMVLKSFN |
| 1561 | DSNLSLFHLQ KSIIKNNIKG RLFLITNGGQ SISSSTPTST YNDQSYVNLS QYQLIGQIRV |
| 1621 | FSNEYPIMEC SMIDIQDSTR IDLITDQLNS TKLSKLEIAF RDNIGYSYKL LKPSIFDNSS |
| 1681 | LPSSSSEIET TATTKDEEKN NSINYNNNYY RVELSDNGII SDLKIKQFRQ MKCGVGQVLV |
| 1741 | RVEMCTLNFR DILKSLGRDY DPIHLNSMGD EFSGKVIEIG EGVNNLSVGQ YVFGINMSKS |
| 1801 | MGSFVCCNSD LVFPIPIPTP SSSSSSNENI DDQEIISKLL NQYCTIPIVF LTSWYSIVIQ |
| 1861 | GRLKKGEKIL IHSGCGGVGL ATIQISMMIG AEIHVTVGSN EKKQYLIKEF GIDEKRIYSS |
| 1921 | RSLQFYNDLM VNTDGQGVDM VLNSLSGEYL EKSIQCLSQY GRFIEIGKKD IYSNSSIHLE |
| 1981 | PFKNNLSFFA VDIAQMTENR RDYLREIMID QLLPCFKNGS LKPLNQHCFN SPCDLVKAIR |
| 2041 | FMSSGNHIGK ILINWSNLNN DKQFINHHSV VHLPIQSFSN RSTYIFTGFG GLTQTLLKYF |
| 2101 | STESDLTNVI IVSKNGLDDN SGSGSGNNEK LKLINQLKES GLNVLVEKCD LSSIKQVYKL |
| 2161 | FNKIFDNDAS GSDSGDFSDI KGIFHFASLI NDKRILKHNL ESFNYVYNSK ATSAWNLHQV |
| 2221 | SLKYNLNLDH FQTIGSVITI LGNIGQSNYT CANRFVEGLT HLRIGMGLKS SCIHLASIPD |
| 2281 | VGMASNDNVL NDLNSMGFVP FQSLNEMNLG FKKLLSSPNP IVVLGEINVD RFIEATPNFR |
| 2341 | AKDNFIITSL FNRIDPLLLV NESQDFIINN NINNNGGGGD GSFDDLNQLE DEGQQGFGNG |
| 2401 | DGYVDDNIDS VSMLSGTSSI FDNDFYTKSI RGMLCDILEL KDKDLNNTVS FSDYGLDSLL |
| 2461 | SSELSNTIQK NFSILIPSLT LVDNSTINST VELIKNKLKN STTSSISSSV SKKVSFKKNT |
| 2521 | QPLIIPTTAP ISIIKTQSYI KSEIIESLPI SSSTTIKPLV FDNLVYSSSS SNNSNSKNEL |
| 2581 | TSPPPSAKRE SVLPIISEDN NSDNDSSMAT VIYEISPIAA PYHRYQTDVL KEITQLTPHK |
| 2641 | EFIDNIYKKS KIRSRYCFND FSEKSMADIN KLDAGERVAL FREQTYQTVI NAGKTVIERA |
| 2701 | GIDPMLISHV VGVTSTGIMA PSFDVVLIDK LGLSINTSRT MINFMGCGAA VNSMRAATAY |
| 2761 | AKLKPGTFVL VVAVEASATC MKFNFDSRSD LLSQAIFTDG CVATLVTCQP KSSLVGKLEI |
| 2821 | IDDLSYLMPD SRDALNLFIG PTGIDLDLRP ELPIAINRHI NSAITSWLKK NSLQKSDIEF |
| 2881 | FATHPGGAKI ISAVHEGLGL SPEDLSDSYE VMKRYGNMIG VSTYYVLRRI LDKNQTLLQE |
| 2941 | GSLGYNYGMAMAFSPGASIE AILFKLIK |

SEQ ID NO: 3, Steely1 nucleotide sequence
ATGAATAAAAATTCAAAAATCCAATCACCAAACTCTTCAGATGTAGCAGTAATTGGAGTT
GGTTTTAGATTTCCAGGTAACTCAAACGATCCAGAGTCATTATGGAATAATTTATTAGAT
GGCTTTGATGCTATTACTCAAGTTCCAAAAGAGAGATGGGCTACATCTTTTAGAGAAATG
GGATTAATCAAAAATAAATTTGGTGGTTTTTTAAAAGATTCAGAATGGAAAAATTTTGAT
CCTTTATTTTTTGGAATTGGTCCAAAAGAAGCACCATTTATTGATCCACAACAAAGGTTA
TTATTATCAATTGTTTGGGAATCATTAGAAGATGCATATATTCGTCCAGATGAATTACGT
GGTTCAAATACTGGTGTTTTTATTGGTGTTTCTAATAATGATTATACAAAGTTAGGTTTT
CAAGATAACTATTCAATATCACCTTACACAATGACGGGTTCAAATTCATCATTAAATTCA
AATCGTATTTCATACTGTTTCGATTTCCGTGGACCTTCAATAACCGTTGATACAGCATGC
TCATCTTCATTAGTTTCGGTAAATTTAGGTGTTCAATCGATTCAAATGGGTGAGTGTAAA
ATTGCAATTTGCGGTGGTGTAAATGCACTCTTTGATCCATCAACAAGTGTGGCATTCAGT
AAATTAGGTGTATTAAGTGAAAATGGCCGTTGCAATTCATTCTCTGATCAAGCTTCGGGT
TATGTACGTTCAGAAGGTGCCGGTGTTGTTGTTTTGAAATCATTGGAACAAGCTAAACTC
GACGGTGATAGAATATATGGCGTAATTAAAGGAGTTTCTTCCAATGAAGACGGCGCTTCC
AATGGTGATAAGAATAGTTTAACTACTCCATCTTGTGAAGCTCAATCAATTAATATCTCA
AAAGCAATGGAGAAAGCGTCCTTGTCACCATCCGATATATATTACATTGAGGCTCATGGT
ACAGGTACACCAGTTGGTGATCCAATTGAAGTTAAAGCTTTATCAAAAATATTTAGCAAT
TCAAACAATAATCAATTAAATAATTTTTCCACTGATGGTAACGACAACGACGACGACGAT
GACGATAATACCTCACCAGAACCATTATTAATTGGATCATTTAAATCAAATATTGGTCAT
TTAGAATCAGCTGCTGGAATTGCATCATTAATTAAATGTTGTTTAATGCTTAAAAATCGT
ATGTTAGTTCCATCAATTAATTGTTCAAATTTAAATCCATCAATTCCATTCGATCAATAT
AATATCTCTGTAATTAGAGAAATTAGACAATTTCCAACCGATAAATTGGTAAATATTGGA
ATTAATAGTTTTGGATTTGGAGGTTCAAACTGTCATTTAATAATTCAAGAATATAATAAT
AATTTTAAAAATAATTCAACAATTTGTAATAACAATAATAATAATAATAATAATATAGAT
TATTTAATACCAATTTCAAGTAAAACTAAAAAATCATTAGATAAATATTTAATTTTGATA
AAGACGAATTCAAATTATCATAAAGATATTTCATTTGATGATTTTGTAAAATTTCAAATT
AAATCTAAACAATATAATTTATCAAATAGAATGACTACAATTGCAAACGATTGGAATTCC
TTTATAAAGGGATCAAATGAGTTTCATAATTTAATCGAAAGTAAAGATGGCGAAGGTGGT
AGTAGTAGTAGTAATCGCGGTATTGATAGCGCAAATCAAATCAATACAACTACTACATCA
ACTATAAATGATATTGAACCATTATTAGTATTTGTATTTTGTGGACAAGGACCACAATGG
AATGGAATGATTAAAACATTATATAATAGCGAAAATGTATTCAAGAATACAGTTGATCAT
GTAGATTCAATTTTATATAAATACTTTGGTTATTCAATTTTAAATGTATTATCAAAGATT
GATGATAATGATGATTCAATTAATCATCCAATTGTTGCACAACCATCATTGTTTTTATTA
CAAATTGGTTTAGTTGAATTATTCAAATATTGGGGTATTTATCCATCAATTTCAGTTGGT
CATAGTTTTGGTGAAGTATCATCTTACTATTTATCGGGTATTATTAGTTTAGAGACCGCT
TGTAAAATAGTATATGTAAGAAGTTCAAATCAAAATAAAACAATGGGATCAGGTAAAATG
TTAGTGGTTTCAATGGGTTTTAAACAATGGAATGATCAATTTAGCGCCGAATGGTCAGAT
ATCGAAATCGCTTGTTACAATGCACCAGATTCAATCGTTGTCACAGGTAATGAAGAAAGA
TTAAAAGAATTGTCAATTAAGTTATCCGATGAATCGAATCAAATCTTTAATACATTCTTA
AGATCACCATGTTCATTCCATAGTAGTCACCAAGAAGTTATCAAAGGTTCAATGTTTGAA
GAACTTTCAAATTTACAATCAACTGGTGAAACTGAAATTCCATTATTCTCAACAGTAACT
GGTAGACAAGTCTTGAGTGGTCATGTTACAGCCCAACATATCTATGATAATGTTAGAGAA
CCAGTTTTATTTCAAAAAACAATCGAAAGTATAACATCATATATCAAATCACATTATCCA
TCCAATCAAAAGGTCATTTATGTTGAAATTGCTCCACATCCAACTTTATTTAGTTTAATT
AAAAAATCAATTCCATCATCAAACAAGAATTCTTCATCAGTACTTTGCCCATTGAATAGA
AAAGAGAATTCAAACAATTCATATAAAAAATTTGTTTCTCAATTATACTICAATGGTGTA
AATGTTGATTTCAATTTTCAATTAAATTCAATTTGTGACAATGTTAATAATGATCATCAT
TTGAATAATGTTAAACAAAATTCATTTAAAGAGACAACAAATTCTTTACCAAGATATCAA
TGGGAACAAGATGAATATTGGAGTGAACCATTAATTTCAAGAAAGAATAGATTAGAGGGT
CCAACAACTTCATTGCTTGGTCACAGAATCATTTATTCATTCCCAGTATTTCAAAGTGTT
TTAGATTTACAATCAGATAATTACAAATATTTATTAGATCATTTAGTAAATGGTAAACCA
GTATTCCCAGGTGCTGGTTATTTAGATATAATAATTGAATTCTTTGATTATCAAAAACAA
CAATTGAATTCATCAGATAGTTCAAACTCATATATAATCAATGTTGATAAAATTCAATTC
TTAAACCCAATTCATTTAACTGAGAATAAATTACAAACTCTACAATCATCATTTGAACCA
ATTGTTACTAAAAAGTCAGCATTCTCTGTAAACTTTTTCATAAAGGATACTGTTGAAGAT
CAATCAAAAGTTAAATCAATGAGTGATGAAACTTGGACAAATACTTGTAAAGCAACCATT
TCATTAGAACAACAACAACCATCACCATCATCAACATTAACTTTATCAAAGAAACAAGAT
TTACAAATACTTAGAAATCGTTGTGACATTTCAAAACTTGACAAATTTGAATTGTATGAT
AAGATTTCAAAGAATCTTGGATTACAATATAATTCACTCTTCCAAGTGGTTGATACCATT
GAAACTGGTAAACATTCTTCATTTGCAACACTTTCATTACCAGACGATACTTTATTTACA
ACAATTTTAAATCCATGCCTTTTAGATAATTGTTTCCATGGTTTATTAACTTTAATTAAT
GAAAAAGGTTCATTTGTTGTTGAAAGTATTTCATCAGTTTCAATCTATCTCGAAAATATT
GGTTCATTTAATCAAACATCAGTTGGTAATGTTCAATTCTACCTTTATACTACAATTTCA
AAGGCAACTTCATTCTCATCAGAAGGTACATGTAAATTATTTACAAAAGATGGTAGTTTA
ATTTTATCAATTGGTAAATTTATAATTAAATCAACTAATCCAAAATCAACAAAAACAAAT
GAAACAATTGAATCTCCATTGGATGAAACATTTTCAATTGAATGGCAATCAAAAGATTCA
CCAATTCCAACACCACAACAAATTCAACAACAATCACCATTAAATTCAAATCCATCGTTC
ATTAGATCAACCATTCTTAAGGACATTCAATTTGAACAATATTGRRCTTCAATAATTCAT
AAAGAATTAATTAATCATGAAAAATATAAAAATCAACAATCATTCGATATCAATTCATTG
GAGAATCATTTAAATGATGACCAACTTATGGAATCATTATCAATTTCAAAAGAATATCTT
AGATTCTTTACAAGAATTATTTCAATCATTAAACAATATCCAAAGATATTGAATGAAAAG
GAATTAAAAGAATTAAAAGAAATCATTGAATTAAAGTATCCAAGTGAAGTTCAACTTTTA
GAATTTGAAGTAATTGAAAAAGTTTCAATCATTATTCCAAAATTGTTATTTGAAAATGAT
AAACAATCATCAATGACATTGTTTCAAGATAATCTATTAACTAGATTCTATTCAAATTCA
AATTCAACTCGTTTCTACTTGGAAAGGGTCTCTGAAATGGTGTTAGAATCAATTAGACCA
ATAGTTAGAGAGAAAAGAGTTTTTAGAATTTTAGAAATTGGTGCTGGTACTGGTTCACTT
TCAAATGTTGTTTTAACAAAATTAAATACTTACTTATCAACATTAAATAGTAATGGTGGT
AGCGGTTATAATATAATAATCGAATATACATTTACAGATATTTCAGCAAACTTTATCATT
GGTGAAATTCAAGAGACAATGTGTAACCTTTATCCAAATGTTACATTTAAATTCTCTGTG
TTGGATTTAGAAAAAGAAATCATCAATAGTTCAGATTTCTTAATGGGTGATTATGATATT
GTTTTAATGGCTTATGTAATTCATGCAGTTTCAAATATTAAATTCAGTATTGAACAACTT
TATAAATTATTATCACCAAGAGGTTGGTTATTATGTATTGAACCTAAATCAAATGTTGTC
TTTAGTGATTTAGTTTTTGGTTGTTTCAATCAATGGTGGAATTACTATGATGATATTAGA
ACTACTCATTGTTCATTATCAGAATCACAATGGAACCAATTATTATTAAATCAATCTTTA
AATAATGAATCATCATCATCATCAAATTGTTATGGTGGATTTTCAAATGTATCATTTATT
GGTGGTGAAAAAGATGTAGATTCTCATTCATTTATTTTACATTGTCAAAAAGAATCAATT
TCACAAATGAAATTAGCAACTACAATTAATAATGGTTTATCATCTGGTTCAATTGTAATT
GTTTTAAATAGTCAACAATTAACRAATATGAAATCATACCCAAAGCTTATTGAATATATT
CAAGAGGCAACATCACTTTGTAAAACCATCGAAATTATTGATTCAAAGGATGTTTTAAAT
TCTACAAATTCAGTTTTAGAGAAAATTCAAAAATCTTTATTAGTATTTTGTTTATTAGGA
TATGATTTATTAGAAAATAATTATCAAGAACAATCATTTGAATATGTTAAATTATTAAAT
TTGATTTCAACAACAGCATCATCATCAAATGATAAAAAACCACCAAAGGTATTATTAATT
ACAAAACAAAGTGAAAGAATTTCTAGATCATTCTATTCTAGATCTTTAATTGGTATTTCA
AGAACATCAATGAATGAATATCCAAATTTATCAATTACATCAATTGATTTGGATACAAAT
GATTATTCACTCCAATCATTATTGAAACCAATATTTTCAAATAGTAAATTCTCTGATAAT
GAATTCATCTTTAAGAAGGGATTAATGTTTGTTTCTAGAATTTTCAAGAATAAACAATTA
TTAGAGAGTTCAAATGCATTTGAAACTGATTCTTCAAATTTATATTGTAAAGCATCATCA
GATTTATCATATAAATATGCAATTAAACAATCAATGCTAACTGAAAATCAAATTGAAATT
AAAGTAGAATGCGTTGGTATTAATTTCAAAGATAATCTATTTTACAAAGGTTTATTACCA
CAAGAAATCTTTAGAATGGGTGATATCTATAATCCACCATATGGTTTAGAATGTAGTGGT
GTTATCACTAGAATCGGTTCAAATGTTACTGAATATTCAGTTGGTCAAAATGTTTTTGGA
TTTGCTCGTCATAGTTTAGGTTCACATGTTGTTACCAACAAGGATCTTGTAATCTTAAAA
CCTGATACAATCTCTTTCTCTGAAGCTGCCTCAATTCCGGTAGTTTATTGTACTGCATGG
TATAGTTTATTCAACATTGGTCAATTATCAAATGAAGAAAGCATTTTAATTCATTCAGCA
ACTGGTGGTGTTGGTTTAGCATCATTAAATCTATTGAAAATGAAAAATCAACAACAACAA
CCATTAACAAATGTTTACGCAACAGTTGGATCAAATGAAAAGAAGAAATTTTTAATTGAT
AATTTTAATAATCTTTTCAAAGAAGATGGTGAAAATATTTTTAGTACAAGAGATAAAGAA
TATTCAAATCAATTAGAATCAAAGATTGATGTTATTTTAAATACCTTATCAGGTGAATTT
GTTGAATCAAATTTCAAATCTTTAAGATCTTTTGGAAGACTCATTGATTTATCAGCAACT
CATGTTTATGCAAATCAACAAATTGGTTTAGGTAACTTTAAATTTGATCATCTTTATTCA
GCAGTCGATTTAGAGAGATTAATTGATGAGAAACCAAAACTTCTTCAATCAATTCTTCAA
AGAATTACCAATTCCATTGTAAATGGTAGCCTTGAAAAGATTCCAATTACAATTTTCCCA
TCTACTGAAACTAAAGATGCAATCGAACTCCTATCAAAGAGATCACATATTGGTAAGGTT
GTTGTAGATTGTACAGATATTTCAAAATGTAATCCAGTTGGTGATGTAATTACAAACTTT
TCAATGAGATTACCAAAACCAAACTATCAATTAAATTTAAATTCAACTTTATTGATTACT
GGTCAAAGTGGTTTATCAATCCCATTATTGAATTGGTTATTAAGTAAATCTGGTGGTAAT
GTTAAGAATGTTGTAATCATTTCAAAATCAACAATGAAATGGAAATTACAAACCATGATA
AGTCATTTCGTATCAGGATTTGGTATTCACTTTAACTATGTTCAAGTTGATATTTCAAAC
TACGATGCCTTATCGGAGGCAATCAAGCAATTACCATCCGATTTACCACCAATTACATCG
GTTTTCCATTTAGCTGCAATTTATAATGATGTACCAATGGATCAAGTTACAATGTCAACC
GTTGAATCAGTTCATAATCCAAAGGTATTGGGCGCTGTTAATCTTCATAGAATTAGTGTT
TCATTTGGTTGGAAATTAAATCATTTCGTATTATTTAGTTCAATTACTGCCATCACTGGT
TATCCCGATCAATCAATTTACAATTCAGCCAATAGTATTTTAGATGCACTTTCAAATTTC
CGTAGATTCATGGGATTACCATCATTCTCTATTAATTTAGGTCCAATGAAGGATGAAGGT
AAAGTTTCAACCAATAAATCCATTAAAAAACTATTCAAAAGTCGTGGTTTACCATCATTA
TCTTTGAATAAATTATTTGGTTTATTAGAAGTTGTTATTAATAACCCATCAAATCATGTA
ATTCCAAGTCAATTAATTTGCTCTCCAATTGATTTTAAAACTTATATTGAATCATTTTCA
ACTATGCGTCCAAAATTATTACATCTTCAACCAACAATTTCAAAACAACAATCATCAATT
ATAAATGATTCAACCAAAGCAAGTTCAAACATATCATTACAAGATAAAATTACTTCAAAA
GTTTCTGATTTATTATCAATTCCAATCTCTAAAATTAATTTTGATCATCCTTTAAAACAT
TATGGTCTTGATTCATTATTAACCGTTCAATTTAAATCATGGATTGACAAAGAATTTGAA
AAGAATTTATTCACCCATATTCAATTAGCAACTATTTCAATTAATTCTTTCCTTGAAAAA
GTTAATGGTTTATCAACTAATAATAATAATAATAATAATAGTAATGTTAAATCATCACCA
TCAATAGTAAAAGAAGAAATTGTTACTTTAGATAAAGATCAACAACCATTATTATTAAAA
GAACATCAACATATTATAATTTCACCAGATATTAGAATTAATAAGCCAAAACGTGAAAGT
TTAATTAGAACTCCAATTCTTAATAAGTTTAATCAAATTACAGAATCAATAATTACCCCT
TCGACACCATCACTATCACAATCAGATGTATTGAAAACTCCACCAATTAAAAGTTTAAAC
AATACAAAGAATTCATCATTAATTAACACACCACCAATTCAAAGTGTACAACAACATCAA
AAACAACAACAAAAAGTTCAAGTAATTCAACAACAACAACAACCATTATCAAGACTCTCA
TATAAATCCAATAATAATTCATTCGTTTTGGGTATTGGTATATCAGTACCAGGTGAACCA
ATTTCTCAACAATCATTGAAAGACTCCATATCGAATGATTTCTCTGACAAAGCTGAGACC
AATGAAAAAGTTAAGAGAATCTTTGAACAATCACAAATTAAAACCCGTCATTTGGTTAGA
GATTATACAAAACCAGAAAACTCTATCAAATTCCGTCATTTGGAAACAATAACCGATGTA
AATAATCAATTCAAGAAAGTTGTACCAGATCTAGCTCAACAAGCATGTTTACGTGCCCTC
AAAGATTGGGGTGGTGACAAAGGTGATATCACTCACATCGTATCTGTTACATCAACTGGT
ATTATCATACCAGATGTTAATTTCAAGTTAATCGACCTTTTAGGTTTAAATAAAGATGTA
GAAAGAGTAAGTTTAAATTTAATGGGCTGTCTCGCTGGTCTTTCAAGTTTAAGAACCGCT
GCTTCATTGGCAAAAGCATCACCACGTAATCGTATCTTGGTGGTTTGTACTGAAGTTTGT
TCATTACATTTCTCAAATACTGATGGTGGTGATCAAATGGTTGCAAGTTCAATCTTTGCA
GATGGTTCTGCCGCTTATATCATTGGTTGTAATCCAAGAATTGAAGAAACACCACTCTAT
GAAGTAATGTGTTCAATCAATCGTTCCTTTCCAAACACTGAAAATGCTATGGTTTGGGAC
CTTGAAAAAGAAGGTTGGAATTTAGGTTTAGATGCTTCCATTCCAATTGTAATCGGTTCA
GGTATTGAAGCTTTCGTAGATACCCTATTGGACAAAGCTAAATTACAAACCTCCACTGCT
ATTTCAGCAAAAGATTGTGAATTTTTAATTCATACTGGTGGTAAATCAATTTTAATGAAT
ATCGAAAATAGTTTAGGTATTGATCCAAAACAAACTAAAAACACTTGGGATGTATATCAT
GCATATGGCAATATGTCAAGTGCTTCCGTTATCTTTGTAATGGATCATGCAAGAAAATCA
AAATCATTACCAACTTATTCAATCTCTTTAGCCTTTGGTCCTGGTTTAGCTTTTGAAGGT
TGTTTCTTAAAAAATGTTGTCTAA
SEQ ID NO: 4, Steely2 nucleotide sequence
ATGAACAACAACAAAAGTATAAACGATTTAAGTGGTAATAGCAACAACAACATTGCAAAC
AGTAATATTAATAATTATAATAATTTAATTAAAAAGGAACCAATTGCAATTATTGGAATT
GGTTGCAGATTCCCAGGAAACGTTTCAAATTATTCCGATTTTGTTAATATAATTAAAAAT
GGTAGTGATTGTTTAACTAAAATTCCAGATGATAGATGGAATGCTGATATAATTTCAAGA
AAACAATGGAAATTAAATAATAGAATTGGCGGTTATTTAAAGAATATCGATCAATTTGAT
AATCAATTTTTTGGAATCTCACCAAAAGAAGCTCAACATATTGATCCACAACAAAGATTA
TTATTACATCTTGCAATTGAAACATTAGAAGATGGAAAAATTAGTTTAGATGAAATTAAA
GGTAAAAAAGTTGGAGTTTTTATTGGATCATCAAGTGGAGATTATTTGAGAGGATTTGAT
TCAAGTGAAATTAATCAATTCACAACACCAGGAACCAATTCATCATTTTTAAGTAATAGA
TTATCCTATTTTTTAGATGTTAATGGACCAAGTATGACAGTGAATACAGCATGTTCAGCA
TCAATGGTAGCAATTCATTTAGGATTACAATCACTATGGAATGGTGAAAGTGAATTGTCA
ATGGTTGGTGGAGTGAATATTATTAGCTCACCGCTACAATCGTTGGATTTCGGTAAAGCA
GGTTTACTAAATCAAGAGACCGATGGCAGGTGCTACTCTTTTGATCCACGTGCATCTGGA
TATGTTAGATCCGAAGGTGGAGGAATACTACTATTGAAGCCTTTATCCGCTGCCCTCAGA
GACAATGATGAAATCTATTCATTACTTTTAAACTCTGCAAACAACTCCAATGGTAAAACA
CCAACTGGTATCACCTCACCAAGATCACTATGTCAAGAGAAATTGATTCAACAATTACTA
AGAGAATCGTCAGACCAATTTAGTATTGACGATATTGGCTATTTCGAATGTCATGGTACA
GGCACACAAATGGGTGACCTCAATGAAATCACAGCAATTGGTAAATCGATTGGTATGTTA
AAATCTCACGATGATCCATTGATCATTGGTAGTGTGAAAGCCTCGATTGGCCATCTTGAG
GGTGCAAGTGGTATTTGTGGTGTCATTAAATCAATCATTTGTTTAAAAGAGAAAATCTTA
CCACAACAATGTAAATTCTCTTCTTATAATCCAAAAATACCATTTGAAACTTTAAATTTA
AAAGTTTTAACAAAAACCCAACCTTGGAATAATTCAAAAAGAATTTGTGGTGTAAATTCA
TTTGGTGTTGGTGGTTCAAATTCAAGTTTATTTTTATCATCATTTGATAAATCAACAACA
ATAACAGAACCAACAACAACAACAACAATTGAATCATTACCATCATCGTCATCATCTTTT
GATAATTTATCAGTATCAAGTTCAATATCAACAAATAATGATAATGATAAAGTTAGCAAT
ATTGTTAACAATAGATATGGCAGTAGTATTGATGTTATTACGTTATCAGTTACATCACCA
GATAAAGAAGATTTAAAGATTAGAGCAAATGATGTTTTAGAATCAATTAAAACTTTAGAT
GATAATTTTAAAATTAGAGATATTTCAAATTTAACAAATATTAGAACAAGTCATTTTTCA
AATAGAGTTGCCATCATTGGTGATTCAATCGATTCAATTAAATTAAATTTACAATCATTT
ATTAAGGGTGAAAATAATAATAATAAATCAATAATATTACCTTTAATTAATAATGGTAAT
AATAATAATAATAATAATAATAATAGTAGTGGTAGTAGTAGTAGTAGTAGTAATAATAAT
AATATTTGTTTTATATTTTCAGGTCAAGGTCAACAATGGAATAAAATGATATTCGATTTA
TATGAAAATAATAAAACATTTAAAAATGAAATGAATAATTTTAGTAAACAATTTGAAATG
ATTTCAGGTTGGTCAATTATTGATAAATTATATAATAGTGGTGGTGGTGGTAATGAAGAA
TTAATTAATGAAACTTGGTTAGCACAACCATCAATTGTTGCAGTTCAATATTCATTAATT
AAATTATTTTCAAAAGATATTGGTATTGAAGGTTCAATTGTGTTGGGACATAGTTTAGGT
GAATTGATGGCAGCTTATTATTGTGGTATCATTAATGATTTCAATGATCTATTGAAATTG
TTATATATTAGATCAACACTTCAAAATAAAACCAATGGTAGTGGAAGAATGCATGTTTGT
TTATCTTCAAAAGCAGAGATTGAACAATTGATCTCTCAATTAGGATTCAATGGTAGAATC
GTAATTTGTGGTAATAACACCATGAAATCATGTACAATCTCTGGTGATAATGAATCAATG
AATCAATTCACAAAGTTAATATCATCACAACAGTATGGTTCGGTGGTGCATAAAGAGGTT
CGTACAAATTCAGCATTTCATTCTCATCAAATGGATATTATCAAAGATGAATTCTTTAAA
TTGTTTAATCAATACTTTCCAACCAACCAAATCAGTACAAATCAAATCTACGATGGTAAA
TCATTTTATTCAACTTGTTATGGTAAATATTTAACACCGATTGAATGTAAACAATTATTA
TCATCACCAAATTATTGGTGGAAAAATATCAGAGAATCAGTATTATTCAAAGAATCAATT
GAACAAATCTTACAAAATCATCAACAATCTTTAACATTTATTGAAATTACTTGTCATCCA
ATTTTAAATTATTTTTTAAGTCAATTATTAAAATCATCAAGTAAATCAAACACATTACTT
TTATCAACACTTTCAAAGAATTCAAATTCAATTGATCAATTATTAATATTATGTTCAAAA
TTATATGTTAATAATTTATCATCAATTAAATGGAATTGGTTTTATGATAAACAACAACAA
CAGCAATCAGAAAGTTTAGTATCATCAAATTTTAAATTACCAGGTAGAAGATGGAAACTT
GAAAAATATTGGATTGAAAATTGTCAAAGACAAATGGATAGAATTAAACCACCAATGTTT
ATATCATTAGATAGAAAGTTATTCTCTGTTACACCATCATTTGAAGTTAGATTAAATCAA
GATAGATTTCAATATTTAAATGATCATCAAATTCAAGATATTCCATTGGTACCATTTTCA
TTCTATATTGAATTGGTTTATGCTTCAATATTTAATTCAATCTCAACTACCACCACCAAC
ACCACAGCATCAACAATGTTTGAAATTGAAAATTTTACAATTGATAGTTCAATTATAATT
GATCAAAAGAAATCAACTTTAATTGGTATTAATTTTAATTCTGATTTAACTAAATTTGAA
ATTGGTAGTATTAATAGCATTGGTAGTGGTAGTAGTAGTAATAATAATTTTATTGAAAAT
AAATGGAAAATTCATTCAAATGGTATAATTAAATATGGTACAAATTATTTAAAATCAAAT
TCAAAATCAAATTCATTTAATGAATCAACAACAACAACAACAACAACAACAACAACAACA
AAATGTTTTAAATCATTTAATTCAAATGAATTTTATAATGAAATTATTAAATATAATTAT
AATTACAAGAGTACTTTTCAATGTGTTAAAGAGTTTAAACAATTTGATAAACAAGGTACA
TTCTATTATTCAGAGATTCAATTCAAAAAGAATGATAAACAAGTCATTGATCAATTATTA
TCAAAACAATTACCAAGTGATTTTAGATGTATTCATCCATGTTTATTAGATGCAGTTTTA
CAATCTGCTATCATACCAGCAACAAATAAAACTAATTGTAGTTGGATACCAATTAAAATT
GGTAAATTATCTGTAAATATACCTTCAAATTCATATTTTAATTTTAAAGATCAATTATTA
TATTGTTTAATTAAACCATCAACATCAACATCAACATCACCATCAACATACTTTTCATCT
GATATTCAAGTATTTGATAAAAAGAATAATAATTTAATTTGTGAATTAACAAATTTAGAA
TTTAAAGGTATTAATTCATCATCATCATCATCATCATCATCATCTACAATAAATTCAAAT
GTTGAAGCTAATTATGAATCAAAAATTGAAGAAACTAATCATGATGAGGATGAGGATGAA
GAATTACCATTAGTTTCAGAATATGTTTGGTGTAAAGAAGAATTAATTAATCAATCAATT
AAATTTACAGATAATTATCAAACTGTTATTTTCTGTTCAACAAATTTAAATGGTAATGAT
TTATTAGATAGTATTATAACAAGTGCATTAGAGAATGGTCATGATGAGAATAAGATATTC
ATTGTTTCACCACCACCAGTCGAATCGGATCAATATAATAATCGTATCATTATAAATTAT
ACAAATAATGAATCTGATTTCGATGCTTTATTCGCAATCATTAATTCAACAACTTCAATC
AGTGGAAAGAGTGGTTTATTTTCAACACGTTTTATCATTTTACCAAATTTTAATTCAATT
ACTTTTTCAAGTGGTAATTCAACTCCATTAATAACTAATGTCAATGGTAATGGTAATGGT
AAGAGTTGTGGTGGTGGTGGTGGTAGTACAAATAACACAATTTCAAATTCATCATCATCA
ATATCAAGTATTGATAATGGTAATAATGAAGATGAAGAAATGGTATTAAAATCATTTAAT
GATTCAAATTTATCATTATTCCATTTACAAAAATCAATTATTAAAAATAATATTAAAGGT
AGATTATTTTTAATTACAAATGGTGGTCAATCAATTTCAAGCTCAACTCCAACCTCAACA
TATAATGATCAATCATATGTTAATCTATCACAATATCAATTAATTGGTCAAATTAGAGTA
TTTTCAAATGAATATCCAATTATGGAATGTTCAATGATTGATATTCAAGATTCAACTAGA
ATTGATTTAATTACTGATCAATTAAATTCAACAAAGTTATCAAAACTTGAAATTGCATTT
AGAGATAATATTGGTTATAGTTATAAATTATTAAAACCATCAATTTTTGATAATTCTTCA
TTGCCATCATCATCATCAGAAATAGAAACAACAGCAACAACAAAAGATGAAGAAAAAAAT
AATTCAATAAATTATAATAATAATTATTATAGAGTTGAATTATCTGATAATGGTATAATT
TCAGATTTAAAGATTAAACAATTTAGACAAATGAAATGTGGTGTTGGTCAAGTTTTAGTT
AGAGTTGAAATGTGTACTTTAAATTTTAGAGATATTCTTAAATCATTAGGTCGTGATTAT
GATCCAATTCATTTAAATTCAATGGGTGATGAATTCTCTGGTAAAGTCATTGAAATTGGT
GAAGGTGTTAATAATTTATCAGTTGGTCAATATGTTTTTGGTATAAATATGTCAAAATCA
ATGGGTAGTTTTGTTTGTTGTAATTCTGATTTAGTATTTCCAATTCCAATTCCAACTCCA
TCATCATCATCATCATCAAATGAAAATATTGATGATCAAGAAATTATTTCAAAATTATTA
AATCAATATTGTACAATACCAATTGTATTTTTAACATCATGGTATAGTATTGTAATTCAA
GGTAGATTAAAAAAAGGTGAGAAAATTTTAATACATTCAGGATGTGGTGGTGTTGGTTTA
GCAACTATTCAAATTTCAATGATGATTGGTGCTGAAATTCATGTTACAGTTGGTTCAAAT
GAAAAGAAACAATATTTAATCAAAGAGTTTGGCATTGATGAGAAGAGAATCTATTCATCA
AGATCATTGCAATTCTATAATGATTTAATGGTGAATACTGATGGTCAAGGTGTTGATATG
GTTTTAAATTCATTGTCTGGTGAATATTTAGAGAAATCAATTCAATGTTTATCCCAGTAT
GGTAGATTCATTGAAATTGGTAAAAAAGATATTTACTCGAATTCAAGTATTCATTTAGAA
CCATTTAAAAATAATTTATCATTTTTCGCAGTTGATATTGCACAAATGACAGAAAATCGT
AGAGATTATCTAAGAGAGATAATGATCGATCAGCTATTACCATGTTTTAAAAATGGTTCT
TTGAAACCATTGAATCAACATTGTTTCAATTCACCTTGTGATCTTGTTAAAGCCATTAGA
TTCATGTCATCCGGTAATCATATTGGTAAAATCTTAATCAATTGGTCCAATTTAAATAAT
GATAAACAATTCATTAATCATCATTCAGTTGTTCATTTACCAATTCAATCATTTTCTAAT
AGATCAACTTATATTTTCACTGGTTTTGGTGGTTTAACTCAAACATTATTAAAATATTTT
TCAACAGAATCTGATTTAACAAATGTTATAATAGTTAGTAAAAATGGTTTAGATGATAAT
AGTGGTAGTGGTAGTGGTAATAATGAAAAATTAAAATTAATTAATCAATTAAAAGAATCT
GGTTTAAATGTATTGGTTGAAAAATGTGATTTGTCATCAATTAAACAAGTTTATAAATTA
TTTAACAAGATTTTTGATAATGATGCTAGTGGTAGTGATAGTGGTGATTTTAGTGATATT
AAAGGTATTTTCCATTTTGCATCATTGATTAATGATAAAAGAATTTTAAAACATAATTTA
GAATCATTTAATTATGTTTATAATAGTAAGGCTACTAGTGCTTGGAATTTACATCAAGTT
TCATTAAAATATAATTTAAATTTGGATCATTTCCAAACTATTGGTTCAGTCATTACAATT
CTTGGTAATATTGGTCAAAGCAATTACACTTGTGCAAATAGATTCGTTGAAGGTTTAACT
CATTTACGTATTGGTATGGGTTTGAAATCAAGTTGTATTCATTTAGCTTCTATACCTGAT
GTTGGTATGGCTTCAAATGATAATGTTTTAAATGATTTAAATTCAATGGGTTTTGTGCCA
TTCCAATCACTCAATGAAATGAATTTAGGTTTTAAGAAATTATTATCATCACCAAATCCA
ATCGTTGTACTTGGTGAAATTAATGTTGATAGATTCATTGAAGCAACTCCAAACTTTAGA
GCAAAAGATAATTTCATTATTACTTCATTATTTAATCGTATTGATCCTTTACTATTAGTA
AATGAAAGTCAAGATTTTATTATTAATAATAATATTAATAATAATGGTGGTGGCGGCGAT
GGTAGTTTTGATGATTTAAATCAATTAGAAGATGAAGGACAACAAGGATTTGGTAATGGT
GATGGTTATGTTGATGATAATATTGATAGTGTTTCAATGCTATCTGGAACATCATCTATT
TTTGATAATGATTTCTATACTAAATCAATTAGAGGTATGCTTTGTGATATTTTAGAATTA
AAAGATAAAGATTTAAATAATACAGTATCATTTAGTGACTATGGTTTAGATTCATTACTA
TCAAGTGAATTATCAAACACAATTCAAAAGAATTTCAGTATATTAATTCCAAGTTTAACT
TTAGTTGATAATTCAACCATTAATTCAACTGTTGAATTAATTAAAAATAAATTAAAGAAT
TCAACAACTTCTTCAATTTCTTCAAGTGTATCTAAAAAAGTTTCATTTAAAAAAAATACT
CAACCATTAATTATACCAACAACAGCACCAATATCAATAATTAAAACACAAAGTTATATC
AAATCTGAAATTATTGAATCATTACCAATTAGTAGTAGTACAACTATTAAACCATTGGTA
TTTGATAATTTAGTTTATAGTAGTAGTAGTAGTAATAATAGTAATTCTAAAAATGAATTA
ACATCACCACCACCAAGTGCAAAGAGAGAATCAGTTTTACCAATAATATCAGAAGATAAT
AATAGTGATAACGATTCGTCAATGCCAACAGTAATTTATGAAATTTCACCAATTGCTGCA
CCATATCATAGATATCAAACTGATGTATTAAAAGAGATTACACAATTAACACCACATAAA
GAGTTTATTGATAATATTTATAAGAAATCAAAGATTAGATCAAGATATTGTTTCAATGAT
TTCTCTGAGAAATCAATGGCTGATATTAATAAATTGGATGCAGGTGAAAGAGTTGCACTC
TTTAGAGAACAAACTTATCAAACAGTTATCAATGCAGGTAAAACAGTGATAGAGAGAGCT
GGTATTGATCCAATGTTAATTAGTCATGTCGTTGGTGTCACTAGTACTGGTATTATGGCA
CCCTCTTTCGATGTGGTACTCATTGATAAATTGGGTCTATCAATTAATACTAGTAGAACT
ATGATCAATTTCATGGGTTGTGGTGCCGCTGTCAATTCAATGAGAGCTGCCACTGCTTAT
GCTAAATTAAAACCTGGTACTTTTGTATTGGTGGTTGCAGTGGAGGCATCGGCAACCTGT
ATGAAATTCAATTTCGATAGTCGTAGTGATCTATTATCACAAGCTATCTTTACCGATGGT
TGTGTAGCTACGTTGGTAACTTGTCAACCAAAATCATCATTAGTTGGTAAATTGGAAATC
ATCGATGACTTGTCCTATTTAATGCCAGATTCAAGAGACGCTTTAAATCTATTCATTGGT
CCAACTGGTATTGATTTAGATTTACGTCCTGAATTACCAATTGCAATCAATAGACATATC
AATAGTGCTATTACAAGTTGGTTGAAAAAGAATTCACTTCAAAAGAGTGATATCGAATTC
TTTGCTACTCATCCTGGTGGTGCTAAAATCATTTCTGCCGTTCATGAAGGGTTAGGTTTA
TCACCAGAAGATCTATCAGATTCTTATGAAGTTATGAAAAGATATGGTAATATGATAGGT
GTTTCAACTTATTATGTTTTACGTAGAATTTTAGATAAAAATCAAACATTACTTCAAGAA
GGTTCTTTAGGTTATAATTATGGTATGGCTATGGCCTTTTCACCTGGTGCTTCAATTGAA
GCAATTTTATTTAAATTAATTAAATAA

### BIBLIOGRAPHY

1. Strmecki L, Greene D M, Pears C J. Developmental decisions in Dictyostelium discoideum. Dev Biol 2005; 284(1):25-36.
2. Thompson C R, Kay R R. The role of DIF-1 signaling in Dictyostelium development. Mol Cell 2000; 6(6):1509-14.
3. Kay R R. The biosynthesis of differentiation-inducing factor, a chlorinated signal molecule regulating Dictyostelium development. J Biol Chem 1998; 273(5):2669-75.
4. Austin M B, Noel J P. The chalcone synthase superfamily of type III polyketide synthases. Nat Prod Rep 2003; 20:79-110.
5. Eichinger L, Pachebat J A, Glockner G, Rajandream M A, Sucgang R, Berriman M, et al. The genome of the social amoeba Dictyostelium discoideum. Nature 2005; 435(7038):43-57.
6. Guigo R, Knudsen S, Drake N, Smith T. Prediction of gene structure. J Mol Biol 1992; 226(1):141-57.
7. Morio T, Urushihara H, Saito T, Ugawa Y, Mizuno H, Yoshida M, et al. The Dictyostelium developmental cDNA project: generation and analysis of expressed sequence tags from the first-finger stage of development. DNA Res 1998; 5(6):335-40.
8. Rangan V S, Joshi A K, Smith S. Mapping the functional topology of the animal fatty acid synthase by mutant complementation in vitro. Biochemistry 2001; 40(36):10792-9.
9. Khosla C, Gokhale R S, Jacobsen J R, Cane D E. Tolerance and specificity of polyketide synthases. Annu Rev Biochem 1999; 68:219-53.
10. Staunton J, Weissman K J. Polyketide biosynthesis: a millennium review. Nat Prod Rep 2001; 18(4):380-416.
11. Shen B. Biosynthesis of Aromatic Polyketides. In: Biosynthesis: aromatic polyketides, isoprenoids, alkaloids. Berlin N.Y.: Springer; 2000. p. 1-51.
12. Seshime Y, Juvvadi P R, Fujii I, Kitamoto K. Discovery of a novel superfamily of type III polyketide synthases in Aspergillus oryzae. Biochem Biophys Res Commun 2005; 331(1):253-60.
13. Ferrer J L, Jez J M, Bowman M E, Dixon R A, Noel J P. Structure of chalcone synthase and the molecular basis of plant polyketide biosynthesis. Nat Struct Biol 1999; 6(8):775-84.
14. Jez J M, Austin M B, Ferrer J, Bowman M E, Schroder J, Noel J P. Structural control of polyketide formation in plant-specific polyketide synthases. Chem Biol 2000; 7(12):919-30.
15. Austin M B, Bowman M E, Ferrer J, Schroder J, Noel J P. An aldol switch discovered in stilbene synthases mediates cyclization specificity of type III polyketides synthases. Chem Biol 2004; 11(9):1179-94.
16. Austin M B, Izumikawa M, Bowman M E, Udwary D W, Ferrer J L, Moore B S, et al. Crystal structure of a bacterial type III polyketide synthase and enzymatic control of reactive polyketide intermediates. J Biol Chem 2004; 279(43):45162-74.
17. Sankaranarayanan R, Saxena P, Marathe U B, Gokhale R S, Shanmugam V M, Rukmini R. A novel tunnel in mycobacterial type III polyketide synthase reveals the structural basis for generating diverse metabolites. Nat Struct Mol Biol 2004; 11(9):894-900.
18. Winkel B S. Metabolic channeling in plants. Annu Rev Plant Biol 2004; 55:85-107.
19. Morris H R, Masento M S, Taylor G W, Jermyn K A, Kay R R. Structure elucidation of two differentiation inducing factors (DIF-2 and DIF-3) from the cellular slime mould Dictyostelium discoideum. Biochem J 1988; 249(3):903-6.
20. Serafimidis I, Kay R R. New prestalk and prespore inducing signals in Dictyostelium. Dev Biol 2005; 282(2):432-41.
21. Takaya Y, Kikuchi H, Terui Y, Komiya J, Furukawa K I, Seya K, et al. Novel acyl alpha-pyronoids, dictyopyrone A, B, and C, from Dictyostelium cellular slime molds. J Org Chem 2000; 65(4):985-9.
22. Chirala S S, Wakil S J. Structure and function of animal fatty acid synthase. Lipids 2004; 39(11):1045-53.
23. Tsai S C, Miercke U, Krucinski J, Gokhale R, Chen J C, Foster P G, et al. Crystal structure of the macrocycle-forming thioesterase domain of the erythromycin polyketide synthase: versatility from a unique substrate channel. Proc Natl Acad Sci USA 2001; 98(26):14808-13.
24. Abe I, Utsumi Y, Oguro S, Noguchi H. The first plant type III polyketide synthase that catalyzes formation of aromatic heptaketide. FEBS Lett 2004; 562(1-3):171-176.
25. Abe I, Utsumi Y, Oguro S, Morita H, Sano Y, Noguchi H. A plant type III polyketide synthase that produces pentaketide chromone. J Am Chem Soc 2005; 127(5):1362-3.
26. Jez J M, Ferrer J L, Bowman M E, Dixon R A, Noel J P. Dissection of malonyl-coenzyme A decarboxylation from polyketide formation in the reaction mechanism of a plant polyketide synthase. Biochemistry 2000; 39(5):890-902.
27. Otwinowski Z, and Minor, W. Processing of X-ray diffraction data collected in oscillation mode. Methods Enzymol 1997; 276:307-326.
28. Dodson E J, Winn, M., Ralph, A. Collaborative Computational Project, Number 4: providing programs for protein crystallography. Methods Enzymol 1997; 277:620-633.
29. McCoy A J, Grosse-Kunstleve R W, Storoni L C, Read R J. Likelihood-enhanced fast translation functions. Acta Crystallogr D Biol Crystallogr 2005; 61(Pt 4):458-64.
30. Sali A, and Blundell, T. L. Comparative protein modeling by satisfaction of spatial restraints. J Mol Biol 1993; 234:779-815.
31. Brunger A T, Adams, P. D., Clore, G. M., DeLano, W. L., Gros, P., Grosse-Kunstleve, R. W., Jiang, J. S., Kuszewski, J., Nilges, M., Pannu, N. S., et al. Crystallography and NMR system: a new software suite for macromolecular structure determination. Acta Crystallogr D Biol Crystallogr 1998; 54:905-921.
32. Jones T A, Zou, J. Y., Cowan, S. W., and Kjeldgaard, M. Improved methods for building protein models in electron density maps and the location of errors in these models. Acta Crystallogr D Biol Crystallogr 1993; 49:148-157.
33. Jez J M, Ferrer J L, Bowman M E, Austin M B, Schroder J, Dixon R A, et al. Structure and mechanism of chalcone synthase-like polyketide synthases. J Ind Microbiol Biotechnol 2001; 27(6):393-8.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A recombinant fusion protein comprising:
at least one type I polyketide synthase domain or type I fatty acid synthase domain; and
a type III polyketide synthase domain.

2. The recombinant fusion protein of claim 1, wherein the at least one type I polyketide or fatty acid synthase domain comprises one or more of: a ketoacyl synthase domain, an acyl transferase domain, a dehydratase domain, an enoyl reductase domain, a ketoreductase domain, and an acyl carrier domain.

3. The recombinant fusion protein of claim 1, comprising type I fatty acid synthase ketoacyl synthase, acyl transferase, dehydratase, enoyl reductase, ketoreductase, and acyl carrier domains.

4. The recombinant fusion protein of claim 1, wherein the type III polyketide synthase domain is C-terminal to the at least one type I polyketide synthase domain or type I fatty acid synthase domain.

5. The recombinant fusion protein of claim 1, wherein the type III polyketide synthase domain is selected from the group consisting of: chalcone synthase, stilbene synthase, stilbenecarboxylate synthase, bibenzyl synthase, homoeriodictyol/eriodictyol synthase, acridone synthase, benzophenone synthase, phlorisovalerophenone synthase, coumaroyl triacetic acid synthase, benzalacetone synthase, 1,3,6,8-tetrahydroxynaphthalene synthase, phloroglucinol synthase, dihydroxyphenylacetate synthase, alkylresorcinol synthase, alkyl pyrone synthase, aloesone synthase, pentaketide chromone synthase, and octaketide synthase.

6. The recombinant fusion protein of claim 1, comprising:
a) the amino acid sequence of SEQ ID NO: 1 residues 2776-3147;
b) the amino acid sequence of SEQ ID NO:1 residues 2629-3147;
c) the amino acid sequence of SEQ ID NO:1 residues 2560-3147;
d) the amino acid sequence of SEQ ID NO:2 residues 2616-2968;
e) the amino acid sequence of SEQ ID NO:2 residues 2473-2968;
f) the amino acid sequence of SEQ ID NO:2 residues 2412-2968; or
g) an amino acid sequence at least about 90% identical to the amino acid sequence of any of a-f.

7. The recombinant fusion protein of claim 1, wherein the at least one type I polyketide synthase domain or type I fatty acid synthase domain catalyzes conversion of one or more first precursors to an intermediate, which intermediate is covalently bound to the fusion protein; and wherein the type III polyketide synthase domain catalyzes conversion of the intermediate to a polyketide product.

8. A recombinant fusion protein comprising:
at least a first domain that catalyzes conversion of one or more precursors to an intermediate, which intermediate is covalently bound to the fusion protein; and
a second domain that catalyzes conversion of the intermediate to a product.

9. The recombinant fusion protein of claim 8, wherein when the at least one first domain comprises a type I polyketide synthase domain or a non-ribosomal peptide synthetase domain, the second domain is other than a type I polyketide synthase domain or a nonribosomal peptide synthetase domain.

10. The recombinant fusion protein of claim 8, wherein the product is released by the second domain.

11. The recombinant fusion protein of claim 10, wherein the second domain is other than a thioesterase domain.

12. The recombinant fusion protein of claim 8, wherein the first domain is derived from an enzyme that catalyzes conversion of the one or more precursors to a diffusible product.

13. The recombinant fusion protein of claim 8, wherein the second domain is derived from an enzyme that catalyzes conversion of a diffusible substrate to the product.

14. The recombinant fusion protein of claim 8, wherein the first domain is a type I polyketide synthase domain or type I fatty acid synthase domain; and wherein the fusion protein comprises an acyl carrier domain, to which the intermediate is covalently bound.

15. The recombinant fusion protein of claim 8, wherein the fusion protein comprises an acyl carrier domain, to which the intermediate is covalently bound; and wherein the second domain is selected from the group consisting of: a beta-ketosynthase domain, an aromatic iterative polyketide synthase domain, a type III polyketide synthase domain, a type II polyketide synthase domain, a non-iterative polyketide synthase domain, an HMG-CoA synthetase domain, a ketoacyl-synthase III domain, and a beta-ketoacyl CoA synthase domain.

16. The recombinant fusion protein of claim 8, wherein the first domain is a type I polyketide synthase domain or type I fatty acid synthase domain; wherein the second domain is a type III polyketide synthase domain; wherein the fusion protein comprises an acyl carrier domain, to which the intermediate is covalently bound; and wherein the product is released by the type III polyketide synthase domain.

17.-40. (canceled)

41. A method of making a polyketide product, the method comprising:
contacting one or more first precursors with the recombinant fusion protein of claim 1, whereby the at least one type I polyketide synthase domain or fatty acid synthase domain catalyzes conversion of the one or more first precursors to an intermediate, and the type III polyketide synthase domain catalyzes conversion of the intermediate to a polyketide product.


SEQUENCE LISTING (65 sequences)

SEQ ID NO: 1 - protein, 3147 aa, Dictyostelium discoideum
MNKNSKIQSPNSSDVAVIGVGFRFPGNSNDPESLWNNLLDGFDAITQVPKERWATSFREMGLIKNKFGGFLKDSEWKNFDPLFFGIGPKEAPFIDPQQRLLLSIVWESLEDAYIRPDELRGSNTGVFIGVSNNDYTKLGFQDNYSISPYTMTGSNSSLNSNRISYCFDFRGPSITVDTACSSSLVSVNLGVQSIQMGECKIAICGGVNALFDPSTSVAFSKLGVLSENGRCNSFSDQASGYVRSEGAGVVVLKSLEQAKLDGDRIYGVIKGVSSNEDGASNGDKNSLTTPSCEAQSINISKAMEKASLSPSDIYYIEAHGTGTPVGDPIEVKALSKIFSNSNNNQLNNFSTDGNDNDDDDDDNTSPEPLLIGSFKSNIGHLESAAGIASLIKCCLMLKNRMLVPSINCSNLNPSIPFDQYNISVIREIRQFPTDKLVNIGINSFGFGGSNCHLIIQEYNNNFKNNSTICNNNNNNNNNIDYLIPISSKTKKSLDKYLILIKTNSNYHKDISFDDFVKFQIKSKQYNLSNRMTTIANDWNSFIKGSNEFHNLIESKDGEGGSSSSNRGIDSANQINTTTTSTINDIEPLLVFVFCGQGPQWNGMIKTLYNSENVFKNTVDHVDSILYKYFGYSILNVLSKIDDNDDSINHPIVAQPSLFLLQIGLVELFKYWGIYPSISVGHSFGEVSSYYLSGIISLETACKIVYVRSSNQNKTMGSGKMLVVSMGFKQWNDQFSAEWSDIEIACYNAPDSIVVTGNEERLKELSIKLSDESNQIFNTFLRSPCSFHSSHQEVIKGSMFEELSNLQSTGETEIPLFSTVTGRQVLSGHVTAQHIYDNVREPVLFQKTIESITSYIKSHYPSNQKVIYVEIAPHPTLFSLIKKSIPSSNKNSSSVLCPLNRKENSNNSYKKFVSQLYFNGVNVDFNFQLNSICDNVNNDHHLNNVKQNSFKETTNSLPRYQWEQDEYWSEPLISRKNRLEGPTTSLLGHRIIYSFPVFQSVLDLQSDNYKYLLDHLVNGKPVFPGAGYLDIIIEFFDYQKQQLNSSDSSNSYIINVDKIQFLNPIHLTENKLQTLQSSFEPIVTKKSAFSVNFFIKDTVEDQSKVKSMSDETWTNTCKATISLEQQQPSPSSTLTLSKKQDLQILRNRCDISKLDKFELYDKISKNLGLQYNSLFQVVDTIETGKDCSFATLSLPEDTLFTTILNPCLLDNCFHGLLTLINEKGSFVVESISSVSIYLENIGSFNQTSVGNVQFYLYTTISKATSFSSEGTCKLFTKDGSLILSIGKFIIKSTNPKSTKTNETIESPLDETFSIEWQSKDSPIPTPQQIQQQSPLNSNPSFIRSTILKDIQFEQYCSSIIHKELINHEKYKNQQSFDINSLENHLNDDQLMESLSISKEYLRFFTRIISIIKQYPKILNEKELKELKEIIELKYPSEVQLLEFEVIEKVSMIIPKLLFENDKQSSMTLFQDNLLTRFYSNSNSTRFYLERVSEMVLESIRPIVREKRVFRILEIGAGTGSLSNVVLTKLNTYLSTLNSNGGSGYNIIIEYTFTDISANFIIGEIQETMCNLYPNVTFKFSVLDLEKEIINSSDFLMGDYDIVLMAYVIHAVSNIKFSIEQLYKLLSPRGWLLCIEPKSNVVFSDLVFGCFNQWWNYYDDIRTTHCSLSESQWNQLLLNQSLNNESSSSSNCYGGFSNVSFIGGEKDVDSHSFILHCQKESISQMKLATTINNGLSSGSIVIVLNSQQLTNMKSYPKVIEYIQEATSLCKTIEIIDSKDVLNSTNSVLEKIQKSLLVFCLLGYDLLENNYQEQSFEYVKLLNLISTTASSSNDKKPPKVLLITKQSERISRSFYSRSLIGISRTSMNEYPNLSITSIDLDTNDYSLQSLLKPIFSNSKFSDNEFIFKKGLMFVSRIFKNKQLLESSNAFETDSSNLYCKASSDLSYKYAIKQSMLTENQIEIKVECVGINFKDNLFYKGLLPQEIFRMGDIYNPPYGLECSGVITRIGSNVTEYSVGQNVFGFARHSLGSHVVTNKDLVILKPDTISFSEAASIPVVYCTAWYSLFNIGQLSNEESILIHSATGGVGLASLNLLKMKNQQQQPLTNVYATVGSNEKKKFLIDNFNNLFKEDGENIFSTRDKEYSNQLESKIDVILNTLSGEFVESNFKSLRSFGRLIDLSATHVYANQQIGLGNFKFDHLYSAVDLERLIDEKPKLLQSILQRITNSIVNGSLEKIPITIFPSTETKDAIELLSKRSHIGKVVVDCTDISKCNPVGDVITNFSMRLPKPNYQLNLNSTLLITGQSGLSIPLLNWLLSKSGGNVKNVVIISKSTMKWKLQTMISHFVSGFGIHFNYVQVDISNYDALSEAIKQLPSDLPPITSVFHLAAIYNDVPMDQVTMSTVESVHNPKVLGAVNLHRISVSFGWKLNHFVLFSSITAITGYPDQSIYNSANSILDALSNFRRFMGLPSFSINLGPMKDEGKVSTNKSIKKLFKSRGLPSLSLNKLFGLLEVVINNPSNHVIPSQLICSPIDFKTYIESFSTMRPKLLHLQPTISKQQSSIINDSTKASSNISLQDKITSKVSDLLSIPISKINFDHPLKHYGLDSLLTVQFKSWIDKEFEKNLFTHIQLATISINSFLEKVNGLSTNNNNNNNSNVKSSPSIVKEEIVTLDKDQQPLLLKEHQHIIISPDIRINKPKRESLIRTPILNKFNQITESIITPSTPSLSQSDVLKTPPIKSLNNTKNSSLINTPPIQSVQQHQKQQQKVQVIQQQQQPLSRLSYKSNNNSFVLGIGISVPGEPISQQSLKDSISNDFSDKAETNEKVKRIFEQSQIKTRHLVRDYTKPENSIKFRHLETITDVNNQFKKVVPDLAQQACLRALKDWGGDKGDITHIVSVTSTGIIIPDVNFKLIDLLGLNKDVERVSLNLMGCLAGLSSLRTAASLAKASPRNRILVVCTEVCSLHFSNTDGGDQMVASSIFADGSAAYIIGCNPRIEETPLYEVMCSINRSFPNTENAMVWDLEKEGWNLGLDASIPIVIGSGIEAFVDTLLDKAKLQTSTAISAKDCEFLIHTGGKSILMNIENSLGIDPKQTKNTWDVYHAYGNMSSASVIFVMDHARKSKSLPTYSISLAFGPGLAFEGCFLKNVV

SEQ ID NO: 2 - protein, 2968 aa, Dictyostelium discoideum
MNNNKSINDLSGNSNNNIANSNINNYNNLIKKEPIAIIGIGCRFPGNVSNYSDFVNIIKNGSDCLTKIPDDRWNADIISRKQWKLNNRIGGYLKNIDQFDNQFFGISPKEAQHIDPQQRLLLHLAIETLEDGKISLDEIKGKKVGVFIGSSSGDYLRGFDSSEINQFTTPGTNSSFLSNRLSYFLDVNGPSMTVNTACSASMVAIHLGLQSLWNGESELSMVGGVNIISSPLQSLDFGKAGLLNQETDGRCYSFDPRASGYVRSEGGGILLLKPLSAALRDNDEIYSLLLNSANNSNGKTPTGITSPRSLCQEKLIQQLLRESSDQFSIDDIGYFECHGTGTQMGDLNEITAIGKSIGMLKSHDDPLIIGSVKASIGHLEGASGICGVIKSIICLKEKILPQQCKFSSYNPKIPFETLNLKVLTKTQPWNNSKRICGVNSFGVGGSNSSLFLSSFDKSTTITEPTTTTTIESLPSSSSSFDNLSVSSSISTNNDNDKVSNIVNNRYGSSIDVITLSVTSPDKEDLKIRANDVLESIKTLDDNFKIRDISNLTNIRTSHFSNRVAIIGDSIDSIKLNLQSFIKGENNNNKSIILPLINNGNNNNNNNNNSSGSSSSSSNNNNICFIFSGQGQQWNKMIFDLYENNKTFKNEMNNFSKQFEMISGWSIIDKLYNSGGGGNEELINETWLAQPSIVAVQYSLIKLFSKDIGIEGSIVLGHSLGELMAAYYCGIINDFNDLLKLLYIRSTLQNKTNGSGRMHVCLSSKAEIEQLISQLGFNGRIVICGNNTMKSCTISGDNESMNQFTKLISSQQYGSVVHKEVRTNSAFHSHQMDIIKDEFFKLFNQYFPTNQISTNQIYDGKSFYSTCYGKYLTPIECKQLLSSPNYWWKNIRESVLFKESIEQILQNHQQSLTFIEITCHPILNYFLSQLLKSSSKSNTLLLSTLSKNSNSIDQLLILCSKLYVNNLSSIKWNWFYDKQQQQQSESLVSSNFKLPGRRWKLEKYWIENCQRQMDRIKPPMFISLDRKLFSVTPSFEVRLNQDRFQYLNDHQIQDIPLVPFSFYIELVYASIFNSISTTTTNTTASTMFEIENFTIDSSIIIDQKKSTLIGINFNSDLTKFEIGSINSIGSGSSSNNNFIENKWKIHSNGIIKYGTNYLKSNSKSNSFNESTTTTTTTTTTTKCFKSFNSNEFYNEIIKYNYNYKSTFQCVKEFKQFDKQGTFYYSEIQFKKNDKQVIDQLLSKQLPSDFRCIHPCLLDAVLQSAIIPATNKTNCSWIPIKIGKLSVNIPSNSYFNFKDQLLYCLIKPSTSTSTSPSTYFSSDIQVFDKKNNNLICELTNLEFKGINSSSSSSSSSSTINSNVEANYESKIEETNHDEDEDEELPLVSEYVWCKEELINQSIKFTDNYQTVIFCSTNLNGNDLLDSIITSALENGHDENKIFIVSPPPVESDQYNNRIIINYTNNESDFDALFAIINSTTSISGKSGLFSTRFIILPNFNSITFSSGNSTPLITNVNGNGNGKSCGGGGGSTNNTISNSSSSISSIDNGNNEDEEMVLKSFNDSNLSLFHLQKSIIKNNIKGRLFLITNGGQSISSSTPTSTYNDQSYVNLSQYQLIGQIRVFSNEYPIMECSMIDIQDSTRIDLITDQLNSTKLSKLEIAFRDNIGYSYKLLKPSIFDNSSLPSSSSEIETTATTKDEEKNNSINYNNNYYRVELSDNGIISDLKIKQFRQMKCGVGQVLVRVEMCTLNFRDILKSLGRDYDPIHLNSMGDEFSGKVIEIGEGVNNLSVGQYVFGINMSKSMGSFVCCNSDLVFPIPIPTPSSSSSSNENIDDQEIISKLLNQYCTIPIVFLTSWYSIVIQGRLKKGEKILIHSGCGGVGLATIQISMMIGAEIHVTVGSNEKKQYLIKEFGIDEKRIYSSRSLQFYNDLMVNTDGQGVDMVLNSLSGEYLEKSIQCLSQYGRFIEIGKKDIYSNSSIHLEPFKNNLSFFAVDIAQMTENRRDYLREIMIDQLLPCFKNGSLKPLNQHCFNSPCDLVKAIRFMSSGNHIGKILINWSNLNNDKQFINHHSVVHLPIQSFSNRSTYIFTGFGGLTQTLLKYFSTESDLTNVIIVSKNGLDDNSGSGSGNNEKLKLINQLKESGLNVLVEKCDLSSIKQVYKLFNKIFDNDASGSDSGDFSDIKGIFHFASLINDKRILKHNLESFNYVYNSKATSAWNLHQVSLKYNLNLDHFQTIGSVITILGNIGQSNYTCANRFVEGLTHLRIGMGLKSSCIHLASIPDVGMASNDNVLNDLNSMGFVPFQSLNEMNLGFKKLLSSPNPIVVLGEINVDRFIEATPNFRAKDNFIITSLFNRIDPLLLVNESQDFIINNNINNNGGGGDGSFDDLNQLEDEGQQGFGNGDGYVDDNIDSVSMLSGTSSIFDNDFYTKSIRGMLCDILELKDKDLNNTVSFSDYGLDSLLSSELSNTIQKNFSILIPSLTLVDNSTINSTVELIKNKLKNSTTSSISSSVSKKVSFKKNTQPLIIPTTAPISIIKTQSYIKSEIIESLPISSSTTIKPLVFDNLVYSSSSSNNSNSKNELTSPPPSAKRESVLPIISEDNNSDNDSSMATVIYEISPIAAPYHRYQTDVLKEITQLTPHKEFIDNIYKKSKIRSRYCFNDFSEKSMADINKLDAGERVALFREQTYQTVINAGKTVIERAGIDPMLISHVVGVTSTGIMAPSFDVVLIDKLGLSINTSRTMINFMGCGAAVNSMRAATAYAKLKPGTFVLVVAVEASATCMKFNFDSRSDLLSQAIFTDGCVATLVTCQPKSSLVGKLEIIDDLSYLMPDSRDALNLFIGPTGIDLDLRPELPIAINRHINSAITSWLKKNSLQKSDIEFFATHPGGAKIISAVHEGLGLSPEDLSDSYEVMKRYGNMIGVSTYYVLRRILDKNQTLLQEGSLGYNYGMAMAFSPGASIEAILFKLIK

SEQ ID NO: 3 - DNA, 9444 bp, Dictyostelium discoideum
atgaataaaaattcaaaaatccaatcaccaaactcttcagatgtagcagtaattggagttggttttagatttccaggtaactcaaacgatccagagtcattatggaataatttattagatggctttgatgctattactcaagttccaaaagagagatgggctacatcttttagagaaatgggattaatcaaaaataaatttggtggttttttaaaagattcagaatggaaaaattttgatcctttattttttggaattggtccaaaagaagcaccatttattgatccacaacaaaggttattattatcaattgtttgggaatcattagaagatgcatatattcgtccagatgaattacgtggttcaaatactggtgtttttattggtgtttctaataatgattatacaaagttaggttttcaagataactattcaatatcaccttacacaatgacgggttcaaattcatcattaaattcaaatcgtatttcatactgtttcgatttccgtggaccttcaataaccgttgatacagcatgctcatcttcattagtttcggtaaatttaggtgttcaatcgattcaaatgggtgagtgtaaaattgcaatttgcggtggtgtaaatgcactctttgatccatcaacaagtgtggcattcagtaaattaggtgtattaagtgaaaatggccgttgcaattcattctctgatcaagcttcgggttatgtacgttcagaaggtgccggtgttgttgttttgaaatcattggaacaagctaaactcgacggtgatagaatatatggcgtaattaaaggagtttcttccaatgaagacggcgcttccaatggtgataagaatagtttaactactccatcttgtgaagctcaatcaattaatatctcaaaagcaatggagaaagcgtccttgtcaccatccgatatatattacattgaggctcatggtacaggtacaccagttggtgatccaattgaagttaaagctttatcaaaaatatttagcaattcaaacaataatcaattaaataatttttccactgatggtaacgacaacgacgacgacgatgacgataatacctcaccagaaccattattaattggatcatttaaatcaaatattggtcatttagaatcagctgctggaattgcatcattaattaaatgttgtttaatgcttaaaaatcgtatgttagttccatcaattaattgttcaaatttaaatccatcaattccattcgatcaatataatatctctgtaattagagaaattagacaatttccaaccgataaattggtaaatattggaattaatagttttggatttggaggttcaaactgtcatttaataattcaagaatataataataattttaaaaataattcaacaatttgtaataacaataataataataataataatatagattatttaataccaatttcaagtaaaactaaaaaatcattagataaatatttaattttgataaagacgaattcaaattatcataaagatatttcatttgatgattttgtaaaatttcaaattaaatctaaacaatataatttatcaaatagaatgactacaattgcaaacgattggaattcctttataaagggatcaaatgagtttcataatttaatcgaaagtaaagatggcgaaggtggtagtagtagtagtaatcgcggtattgatagcgcaaatcaaatcaatacaactactacatcaactataaatgatattgaaccattattagtatttgtattttgtggacaaggaccacaatggaatggaatgattaaaacattatataatagcgaaaatgtattcaagaatacagttgatcatgtagattcaattttatataaatactttggttattcaattttaaatgtattatcaaagattgatgataatgatgattcaattaatcatccaattgttgcacaaccatcattgtttttattacaaattggtttagttgaattattcaaatattggggtatttatccatcaatttcagttggtcatagttttggtgaagtatcatcttactatttatcgggtattattagtttagagaccgcttgtaaaatagtatatgtaagaagttcaaatcaaaataaaacaatgggatcaggtaaaatgttagtggtttcaatgggttttaaacaatggaatgatcaatttagcgccgaatggtcagatatcgaaatcgcttgttacaatgcaccagattcaatcgttgtcacaggtaatgaagaaagattaaaagaattgtcaattaagttatccgatgaatcgaatcaaatctttaatacattcttaagatcaccatgttcattccatagtagtcaccaagaagttatcaaaggttcaatgtttgaagaactttcaaatttacaatcaactggtgaaactgaaattccattattctcaacagtaactggtagacaagtcttgagtggtcatgttacagcccaacatatctatgataatgttagagaaccagttttatttcaaaaaacaatcgaaagtataacatcatatatcaaatcacattatccatccaatcaaaaggtcatttatgttgaaattgctccacatccaactttatttagtttaattaaaaaatcaattccatcatcaaacaagaattcttcatcagtactttgcccattgaatagaaaagagaattcaaacaattcatataaaaaatttgtttctcaattatacttcaatggtgtaaatgttgatttcaattttcaattaaattcaatttgtgacaatgttaataatgatcatcatttgaataatgttaaacaaaattcatttaaagagacaacaaattctttaccaagatatcaatgggaacaagatgaatattggagtgaaccattaatttcaagaaagaatagattagagggtccaacaacttcattgcttggtcacagaatcatttattcattcccagtatttcaaagtgttttagatttacaatcagataattacaaatatttattagatcatttagtaaatggtaaaccagtattcccaggtgctggttatttagatataataattgaattctttgattatcaaaaacaacaattgaattcatcagatagttcaaactcatatataatcaatgttgataaaattcaattcttaaacccaattcatttaactgagaataaattacaaactctacaatcatcatttgaaccaattgttactaaaaagtcagcattctctgtaaactttttcataaaggatactgttgaagatcaatcaaaagttaaatcaatgagtgatgaaacttggacaaatacttgtaaagcaaccatttcattagaacaacaacaaccatcaccatcatcaacattaactttatcaaagaaacaagatttacaaatacttagaaatcgttgtgacatttcaaaacttgacaaatttgaattgtatgataagatttcaaagaatcttggattacaatataattcactcttccaagtggttgataccattgaaactggtaaagattgttcatttgcaacactttcattaccagaggatactttatttacaacaattttaaatccatgccttttagataattgtttccatggtttattaactttaattaatgaaaaaggttcatttgttgttgaaagtatttcatcagtttcaatctatctcgaaaatattggttcatttaatcaaacatcagttggtaatgttcaattctacctttatactacaatttcaaaggcaacttcattctcatcagaaggtacatgtaaattatttacaaaagatggtagtttaattttatcaattggtaaatttataattaaatcaactaatccaaaatcaacaaaaacaaatgaaacaattgaatctccattggatgaaacattttcaattgaatggcaatcaaaagattcaccaattccaacaccacaacaaattcaacaacaatcaccattaaattcaaatccatcgttcattagatcaaccattcttaaggacattcaatttgaacaatattgttcttcaataattcataaagaattaattaatcatgaaaaatataaaaatcaacaatcattcgatatcaattcattggagaatcatttaaatgatgaccaacttatggaatcattatcaatttcaaaagaatatcttagattctttacaagaattatttcaatcattaaacaatatccaaagatattgaatgaaaaggaattaaaagaattaaaagaaatcattgaattaaagtatccaagtgaagttcaacttttagaatttgaagtaattgaaaaagtttcaatgattattccaaaattgttatttgaaaatgataaacaatcatcaatgacattgtttcaagataatctattaactagattctattcaaattcaaattcaactcgtttctacttggaaagggtctctgaaatggtgttagaatcaattagaccaatagttagagagaaaagagtttttagaattttagaaattggtgctggtactggttcactttcaaatgttgttttaacaaaattaaatacttacttatcaacattaaatagtaatggtggtagcggttataatataataatcgaatatacatttacagatatttcagcaaactttatcattggtgaaattcaagagacaatgtgtaacctttatccaaatgttacatttaaattctctgtgttggatttagaaaaagaaatcatcaatagttcagatttcttaatgggtgattatgatattgttttaatggcttatgtaattcatgcagtttcaaatattaaattcagtattgaacaactttataaattattatcaccaagaggttggttattatgtattgaacctaaatcaaatgttgtctttagtgatttagtttttggttgtttcaatcaatggtggaattactatgatgatattagaactactcattgttcattatcagaatcacaatggaaccaattattattaaatcaatctttaaataatgaatcatcatcatcatcaaattgttatggtggattttcaaatgtatcatttattggtggtgaaaaagatgtagattctcattcatttattttacattgtcaaaaagaatcaatttcacaaatgaaattagcaactacaattaataatggtttatcatctggttcaattgtaattgttttaaatagtcaacaattaactaatatgaaatcatacccaaaggttattgaatatattcaagaggcaacatcactttgtaaaaccatcgaaattattgattcaaaggatgttttaaattctacaaattcagttttagagaaaattcaaaaatctttattagtattttgtttattaggatatgatttattagaaaataattatcaagaacaatcatttgaatatgttaaattattaaatttgatttcaacaacagcatcatcatcaaatgataaaaaaccaccaaaggtattattaattacaaaacaaagtgaaagaatttctagatcattctattctagatctttaattggtatttcaagaacatcaatgaatgaatatccaaatttatcaattacatcaattgatttggatacaaatgattattcactccaatcattattgaaaccaatattttcaaatagtaaattctctgataatgaattcatctttaagaagggattaatgtttgtttctagaattttcaagaataaacaattattagagagttcaaatgcatttgaaactgattcttcaaatttatattgtaaagcatcatcagatttatcatataaatatgcaattaaacaatcaatgctaactgaaaatcaaattgaaattaaagtagaatgcgttggtattaatttcaaagataatctattttacaaaggtttattaccacaagaaatctttagaatgggtgatatctataatccaccatatggtttagaatgtagtggtgttatcactagaatcggttcaaatgttactgaatattcagttggtcaaaatgtttttggatttgctcgtcatagtttaggttcacatgttgttaccaacaaggatcttgtaatcttaaaacctgatacaatctctttctctgaagctgcctcaattccggtagtttattgtactgcatggtatagtttattcaacattggtcaattatcaaatgaagaaagcattttaattcattcagcaactggtggtgttggtttagcatcattaaatctattgaaaatgaaaaatcaacaacaacaaccattaacaaatgtttacgcaacagttggatcaaatgaaaagaagaaatttttaattgataattttaataatcttttcaaagaagatggtgaaaatatttttagtacaagagataaagaatattcaaatcaattagaatcaaagattgatgttattttaaataccttatcaggtgaatttgttgaatcaaatttcaaatctttaagatcttttggaagactcattgatttatcagcaactcatgtttatgcaaatcaacaaattggtttaggtaactttaaatttgatcatctttattcagcagtcgatttagagagattaattgatgagaaaccaaaacttcttcaatcaattcttcaaagaattaccaattccattgtaaatggtagccttgaaaagattccaattacaattttcccatctactgaaactaaagatgcaatcgaactcctatcaaagagatcacatattggtaaggttgttgtagattgtacagatatttcaaaatgtaatccagttggtgatgtaattacaaacttttcaatgagattaccaaaaccaaactatcaattaaatttaaattcaactttattgattactggtcaaagtggtttatcaatcccattattgaattggttattaagtaaatctggtggtaatgttaagaatgttgtaatcatttcaaaatcaacaatgaaatggaaattacaaaccatgataagtcatttcgtatcaggatttggtattcactttaactatgttcaagttgatatttcaaactacgatgccttatcggaggcaatcaagcaattaccatccgatttaccaccaattacatcggttttccatttagctgcaatttataatgatgtaccaatggatcaagttacaatgtcaaccgttgaatcagttcataatccaaaggtattgggcgctgttaatcttcatagaattagtgtttcatttggttggaaattaaatcatttcgtattatttagttcaattactgccatcactggttatcccgatcaatcaatttacaattcagccaatagtattttagatgcactttcaaatttccgtagattcatgggattaccatcattctctattaatttaggtccaatgaaggatgaaggtaaagtttcaaccaataaatccattaaaaaactattcaaaagtcgtggtttaccatcattatctttgaataaattatttggtttattagaagttgttattaataacccatcaaatcatgtaattccaagtcaattaatttgctctccaattgattttaaaacttatattgaatcattttcaactatgcgtccaaaattattacatcttcaaccaacaatttcaaaacaacaatcatcaattataaatgattcaaccaaagcaagttcaaacatatcattacaagataaaattacttcaaaagtttctgatttattatcaattccaatctctaaaattaattttgatcatcctttaaaacattatggtcttgattcattattaaccgttcaatttaaatcatggattgacaaagaatttgaaaagaatttattcacccatattcaattagcaactatttcaattaattctttccttgaaaaagttaatggtttatcaactaataataataataataataatagtaatgttaaatcatcaccatcaatagtaaaagaagaaattgttactttagataaagatcaacaaccattattattaaaagaacatcaacatattataatttcaccagatattagaattaataagccaaaacgtgaaagtttaattagaactccaattcttaataagtttaatcaaattacagaatcaataattaccccttcgacaccatcactatcacaatcagatgtattgaaaactccaccaattaaaagtttaaacaatacaaagaattcatcattaattaacacaccaccaattcaaagtgtacaacaacatcaaaaacaacaacaaaaagttcaagtaattcaacaacaacaacaaccattatcaagactctcatataaatccaataataattcattcgttttgggtattggtatatcagtaccaggtgaaccaatttctcaacaatcattgaaagactccatatcgaatgatttctctgacaaagctgagaccaatgaaaaagttaagagaatctttgaacaatcacaaattaaaacccgtcatttggttagagattatacaaaaccagaaaactctatcaaattccgtcatttggaaacaataaccgatgtaaataatcaattcaagaaagttgtaccagatctagctcaacaagcatgtttacgtgccctcaaagattggggtggtgacaaaggtgatatcactcacatcgtatctgttacatcaactggtattatcataccagatgttaatttcaagttaatcgaccttttaggtttaaataaagatgtagaaagagtaagtttaaatttaatgggctgtctcgctggtctttcaagtttaagaaccgctgcttcattggcaaaagcatcaccacgtaatcgtatcttggtggtttgtactgaagtttgttcattacatttctcaaatactgatggtggtgatcaaatggttgcaagttcaatctttgcagatggttctgccgcttatatcattggttgtaatccaagaattgaagaaacaccactctatgaagtaatgtgttcaatcaatcgttcctttccaaacactgaaaatgctatggtttgggaccttgaaaaagaaggttggaatttaggtttagatgcttccattccaattgtaatcggttcaggtattgaagctttcgtagataccctattggacaaagctaaattacaaacctccactgctatttcagcaaaagattgtgaatttttaattcatactggtggtaaatcaattttaatgaatatcgaaaatagtttaggtattgatccaaaacaaactaaaaacacttgggatgtatatcatgcatatggcaatatgtcaagtgcttccgttatctttgtaatggatcatgcaagaaaatcaaaatcattaccaacttattcaatctctttagcctttggtcctggtttagcttttgaaggttgtttcttaaaaaatgttgtctaa

SEQ ID NO: 4 - DNA, 8907 bp, Dictyostelium discoideum
atgaacaacaacaaaagtataaacgatttaagtggtaatagcaacaacaacattgcaaacagtaatattaataattataataatttaattaaaaaggaaccaattgcaattattggaattggttgcagattcccaggaaacgtttcaaattattccgattttgttaatataattaaaaatggtagtgattgtttaactaaaattccagatgatagatggaatgctgatataatttcaagaaaacaatggaaattaaataatagaattggcggttatttaaagaatatcgatcaatttgataatcaattttttggaatctcaccaaaagaagctcaacatattgatccacaacaaagattattattacatcttgcaattgaaacattagaagatggaaaaattagtttagatgaaattaaaggtaaaaaagttggagtttttattggatcatcaagtggagattatttgagaggatttgattcaagtgaaattaatcaattcacaacaccaggaaccaattcatcatttttaagtaatagattatcctattttttagatgttaatggaccaagtatgacagtgaatacagcatgttcagcatcaatggtagcaattcatttaggattacaatcactatggaatggtgaaagtgaattgtcaatggttggtggagtgaatattattagctcaccgctacaatcgttggatttcggtaaagcaggtttactaaatcaagagaccgatggcaggtgctactcttttgatccacgtgcatctggatatgttagatccgaaggtggaggaatactactattgaagcctttatccgctgccctcagagacaatgatgaaatctattcattacttttaaactctgcaaacaactccaatggtaaaacaccaactggtatcacctcaccaagatcactatgtcaagagaaattgattcaacaattactaagagaatcgtcagaccaatttagtattgacgatattggctatttcgaatgtcatggtacaggcacacaaatgggtgacctcaatgaaatcacagcaattggtaaatcgattggtatgttaaaatctcacgatgatccattgatcattggtagtgtgaaagcctcgattggccatcttgagggtgcaagtggtatttgtggtgtcattaaatcaatcatttgtttaaaagagaaaatcttaccacaacaatgtaaattctcttcttataatccaaaaataccatttgaaactttaaatttaaaagttttaacaaaaacccaaccttggaataattcaaaaagaatttgtggtgtaaattcatttggtgttggtggttcaaattcaagtttatttttatcatcatttgataaatcaacaacaataacagaaccaacaacaacaacaacaattgaatcattaccatcatcgtcatcatcttttgataatttatcagtatcaagttcaatatcaacaaataatgataatgataaagttagcaatattgttaacaatagatatggcagtagtattgatgttattacgttatcagttacatcaccagataaagaagatttaaagattagagcaaatgatgttttagaatcaattaaaactttagatgataattttaaaattagagatatttcaaatttaacaaatattagaacaagtcatttttcaaatagagttgccatcattggtgattcaatcgattcaattaaattaaatttacaatcatttattaagggtgaaaataataataataaatcaataatattacctttaattaataatggtaataataataataataataataataatagtagtggtagtagtagtagtagtagtaataataataatatttgttttatattttcaggtcaaggtcaacaatggaataaaatgatattcgatttatatgaaaataataaaacatttaaaaatgaaatgaataattttagtaaacaatttgaaatgatttcaggttggtcaattattgataaattatataatagtggtggtggtggtaatgaagaattaattaatgaaacttggttagcacaaccatcaattgttgcagttcaatattcattaattaaattattttcaaaagatattggtattgaaggttcaattgtgttgggacatagtttaggtgaattgatggcagcttattattgtggtatcattaatgatttcaatgatctattgaaattgttatatattagatcaacacttcaaaataaaaccaatggtagtggaagaatgcatgtttgtttatcttcaaaagcagagattgaacaattgatctctcaattaggattcaatggtagaatcgtaatttgtggtaataacaccatgaaatcatgtacaatctctggtgataatgaatcaatgaatcaattcacaaagttaatatcatcacaacagtatggttcggtggtgcataaagaggttcgtacaaattcagcatttcattctcatcaaatggatattatcaaagatgaattctttaaattgtttaatcaatactttccaaccaaccaaatcagtacaaatcaaatctacgatggtaaatcattttattcaacttgttatggtaaatatttaacaccgattgaatgtaaacaattattatcatcaccaaattattggtggaaaaatatcagagaatcagtattattcaaagaatcaattgaacaaatcttacaaaatcatcaacaatctttaacatttattgaaattacttgtcatccaattttaaattattttttaagtcaattattaaaatcatcaagtaaatcaaacacattacttttatcaacactttcaaagaattcaaattcaattgatcaattattaatattatgttcaaaattatatgttaataatttatcatcaattaaatggaattggttttatgataaacaacaacaacagcaatcagaaagtttagtatcatcaaattttaaattaccaggtagaagatggaaacttgaaaaatattggattgaaaattgtcaaagacaaatggatagaattaaaccaccaatgtttatatcattagatagaaagttattctctgttacaccatcatttgaagttagattaaatcaagatagatttcaatatttaaatgatcatcaaattcaagatattccattggtaccattttcattctatattgaattggtttatgcttcaatatttaattcaatctcaactaccaccaccaacaccacagcatcaacaatgtttgaaattgaaaattttacaattgatagttcaattataattgatcaaaagaaatcaactttaattggtattaattttaattctgatttaactaaatttgaaattggtagtattaatagcattggtagtggtagtagtagtaataataattttattgaaaataaatggaaaattcattcaaatggtataattaaatatggtacaaattatttaaaatcaaattcaaaatcaaattcatttaatgaatcaacaacaacaacaacaacaacaacaacaacaacaaaatgttttaaatcatttaattcaaatgaattttataatgaaattattaaatataattataattacaagagtacttttcaatgtgttaaagagtttaaacaatttgataaacaaggtacattctattattcagagattcaattcaaaaagaatgataaacaagtcattgatcaattattatcaaaacaattaccaagtgattttagatgtattcatccatgtttattagatgcagttttacaatctgctatcataccagcaacaaataaaactaattgtagttggataccaattaaaattggtaaattatctgtaaatataccttcaaattcatattttaattttaaagatcaattattatattgtttaattaaaccatcaacatcaacatcaacatcaccatcaacatacttttcatctgatattcaagtatttgataaaaagaataataatttaatttgtgaattaacaaatttagaatttaaaggtattaattcatcatcatcatcatcatcatcatcatctacaataaattcaaatgttgaagctaattatgaatcaaaaattgaagaaactaatcatgatgaggatgaggatgaagaattaccattagtttcagaatatgtttggtgtaaagaagaattaattaatcaatcaattaaatttacagataattatcaaactgttattttctgttcaacaaatttaaatggtaatgatttattagatagtattataacaagtgcattagagaatggtcatgatgagaataagatattcattgtttcaccaccaccagtcgaatcggatcaatataataatcgtatcattataaattatacaaataatgaatctgatttcgatgctttattcgcaatcattaattcaacaacttcaatcagtggaaagagtggtttattttcaacacgttttatcattttaccaaattttaattcaattactttttcaagtggtaattcaactccattaataactaatgtcaatggtaatggtaatggtaagagttgtggtggtggtggtggtagtacaaataacacaatttcaaattcatcatcatcaatatcaagtattgataatggtaataatgaagatgaagaaatggtattaaaatcatttaatgattcaaatttatcattattccatttacaaaaatcaattattaaaaataatattaaaggtagattatttttaattacaaatggtggtcaatcaatttcaagctcaactccaacctcaacatataatgatcaatcatatgttaatctatcacaatatcaattaattggtcaaattagagtattttcaaatgaatatccaattatggaatgttcaatgattgatattcaagattcaactagaattgatttaattactgatcaattaaattcaacaaagttatcaaaacttgaaattgcatttagagataatattggttatagttataaattattaaaaccatcaatttttgataattcttcattgccatcatcatcatcagaaatagaaacaacagcaacaacaaaagatgaagaaaaaaataattcaataaattataataataattattatagagttgaattatctgataatggtataatttcagatttaaagattaaacaatttagacaaatgaaatgtggtgttggtcaagttttagttagagttgaaatgtgtactttaaattttagagatattcttaaatcattaggtcgtgattatgatccaattcatttaaattcaatgggtgatgaattctctggtaaagtcattgaaattggtgaaggtgttaataatttatcagttggtcaatatgtttttggtataaatatgtcaaaatcaatgggtagttttgtttgttgtaattctgatttagtatttccaattccaattccaactccatcatcatcatcatcatcaaatgaaaatattgatgatcaagaaattatttcaaaattattaaatcaatattgtacaataccaattgtatttttaacatcatggtatagtattgtaattcaaggtagattaaaaaaaggtgagaaaattttaatacattcaggatgtggtggtgttggtttagcaactattcaaatttcaatgatgattggtgctgaaattcatgttacagttggttcaaatgaaaagaaacaatatttaatcaaagagtttggcattgatgagaagagaatctattcatcaagatcattgcaattctataatgatttaatggtgaatactgatggtcaaggtgttgatatggttttaaattcattgtctggtgaatatttagagaaatcaattcaatgtttatcccagtatggtagattcattgaaattggtaaaaaagatatttactcgaattcaagtattcatttagaaccatttaaaaataatttatcatttttcgcagttgatattgcacaaatgacagaaaatcgtagagattatctaagagagataatgatcgatcagctattaccatgttttaaaaatggttctttgaaaccattgaatcaacattgtttcaattcaccttgtgatcttgttaaagccattagattcatgtcatccggtaatcatattggtaaaatcttaatcaattggtccaatttaaataatgataaacaattcattaatcatcattcagttgttcatttaccaattcaatcattttctaatagatcaacttatattttcactggttttggtggtttaactcaaacattattaaaatatttttcaacagaatctgatttaacaaatgttataatagttagtaaaaatggtttagatgataatagtggtagtggtagtggtaataatgaaaaattaaaattaattaatcaattaaaagaatctggtttaaatgtattggttgaaaaatgtgatttgtcatcaattaaacaagtttataaattatttaacaagatttttgataatgatgctagtggtagtgatagtggtgattttagtgatattaaaggtattttccattttgcatcattgattaatgataaaagaattttaaaacataatttagaatcatttaattatgtttataatagtaaggctactagtgcttggaatttacatcaagtttcattaaaatataatttaaatttggatcatttccaaactattggttcagtcattacaattcttggtaatattggtcaaagcaattacacttgtgcaaatagattcgttgaaggtttaactcatttacgtattggtatgggtttgaaatcaagttgtattcatttagcttctatacctgatgttggtatggcttcaaatgataatgttttaaatgatttaaattcaatgggttttgtgccattccaatcactcaatgaaatgaatttaggttttaagaaattattatcatcaccaaatccaatcgttgtacttggtgaaattaatgttgatagattcattgaagcaactccaaactttagagcaaaagataatttcattattacttcattatttaatcgtattgatcctttactattagtaaatgaaagtcaagattttattattaataataatattaataataatggtggtggcggcgatggtagttttgatgatttaaatcaattagaagatgaaggacaacaaggatttggtaatggtgatggttatgttgatgataatattgatagtgtttcaatgctatctggaacatcatctatttttgataatgatttctatactaaatcaattagaggtatgctttgtgatattttagaattaaaagataaagatttaaataatacagtatcatttagtgactatggtttagattcattactatcaagtgaattatcaaacacaattcaaaagaatttcagtatattaattccaagtttaactttagttgataattcaaccattaattcaactgttgaattaattaaaaataaattaaagaattcaacaacttcttcaatttcttcaagtgtatctaaaaaagtttcatttaaaaaaaatactcaaccattaattataccaacaacagcaccaatatcaataattaaaacacaaagttatatcaaatctgaaattattgaatcattaccaattagtagtagtacaactattaaaccattggtatttgataatttagtttatagtagtagtagtagtaataatagtaattctaaaaatgaattaacatcaccaccaccaagtgcaaagagagaatcagttttaccaataatatcagaagataataatagtgataacgattcgtcaatggcaacagtaatttatgaaatttcaccaattgctgcaccatatcatagatatcaaactgatgtattaaaagagattacacaattaacaccacataaagagtttattgataatatttataagaaatcaaagattagatcaagatattgtttcaatgatttctctgagaaatcaatggctgatattaataaattggatgcaggtgaaagagttgcactctttagagaacaaacttatcaaacagttatcaatgcaggtaaaacagtgatagagagagctggtattgatccaatgttaattagtcatgtcgttggtgtcactagtactggtattatggcaccctctttcgatgtggtactcattgataaattgggtctatcaattaatactagtagaactatgatcaatttcatgggttgtggtgccgctgtcaattcaatgagagctgccactgcttatgctaaattaaaacctggtacttttgtattggtggttgcagtggaggcatcggcaacctgtatgaaattcaatttcgatagtcgtagtgatctattatcacaagctatctttaccgatggttgtgtagctacgttggtaacttgtcaaccaaaatcatcattagttggtaaattggaaatcatcgatgacttgtcctatttaatgccagattcaagagacgctttaaatctattcattggtccaactggtattgatttagatttacgtcctgaattaccaattgcaatcaatagacatatcaatagtgctattacaagttggttgaaaaagaattcacttcaaaagagtgatatcgaattctttgctactcatcctggtggtgctaaaatcatttctgccgttcatgaagggttaggtttatcaccagaagatctatcagattcttatgaagttatgaaaagatatggtaatatgataggtgtttcaacttattatgttttacgtagaattttagataaaaatcaaacattacttcaagaaggttctttaggttataattatggtatggctatggccttttcacctggtgcttcaattgaagcaattttatttaaattaattaaataa

SEQ ID NO: 5 - protein, 389 aa, Medicago sativa
MVSVSEIRKAQRAEGPATILAIGTANPANCVEQSTYPDFYFKITNSEHKTELKEKFQRMCDKSMIKRRYMYLTEEILKENPNVCEYMAPSLDARQDMVVVEVPRLGKEAAVKAIKEWGQPKSKITHLIVCTTSGVDMPGADYQLTKLLGLRPYVKRYMMYQQGCFAGGTVLRLAKDLAENNKGARVLVVCSEVTAVTFRGPSDTHLDSLVGQALFGDGAAALIVGSDPVPEIEKPIFEMVWTAQTIAPDSEGAIDGHLREAGLTFHLLKDVPGIVSKNITKALVEAFEPLGISDYNSIFWIAHPGGPAILDQVEQKLALKPEKMNATREVLSEYGNMSSACVLFILDEMRKKSTQNGLKTTGEGLEWGVLFGFGPGLTIETVVLRSVAI

SEQ ID NO: 6 - protein, 6 aa, Artificial
  synthetic consensus sequence
VTSTGX

SEQ ID NO: 7 - protein, 7 aa, Artificial
  synthetic consensus sequence
LIDLLGL

SEQ ID NO: 8 - protein, 6 aa, Artificial
  synthetic consensus sequence
RXLVVC

SEQ ID NO: 9 - protein, 4 aa, Artificial
  synthetic consensus sequence
QAIF

SEQ ID NO: 10 - protein, 6 aa, Artificial
  synthetic consensus sequence
IXGCXP

SEQ ID NO: 11 - protein, 4 aa, Artificial
  synthetic consensus sequence
HPGG

SEQ ID NO: 12 - protein, 9 aa, Artificial
  synthetic consensus sequence
YGNMSSASV

SEQ ID NO: 13 - protein, 6 aa, Artificial
  synthetic consensus sequence
AFGPGL

SEQ ID NO: 14 - protein, 2255 aa, Homo sapiens
MEEVVIAGMSGKLPESENLQEFWDNLIGGVDMVTDDDRRWKAGLYGLPRRSGKLKDLSRFDASFFGVHPKQAHTMDPQLRLLLEVTYEAIVDGGINPDSLRGTHTGVWVGVSGSETSEALSRDPETLVGYSMVGCQRAMMANRLSFFFDFRGPSIALDTACSSSLMALQNAYQAIHSGQCPAAIVGGINVLLKPNTSVQFLRLGMLSPEGTCKAFDTAGNGYCRSEGVVAVLLTKKSLARRVYATILNAGTNTDGFKEQGVTFPSGDIQEQLIRSLYQSAGVAPESFEYIEAHGTGTKVGDPQELNGITRALCATRQEPLLIGSTKSNMGHPEPASGLAALAKVLLSLEHGLWAPNLHFHSPNPEIPALLDGRLQVVDQPLPVRGGNVGINSFGFGGSNVHIILRPNTQPPPAPAPHATLPRLLRASGRTPEAVQKLLEQGLRHSQDLAFLSMLNDIAAVPATAMPFRGYAVLGGERGGPEVQQVPAGERPLWFICSGMGTQWRGMGLSLMRLDRFRDSILRSDEAVKPFGLKVSQLLLSTDESTFDDIVHSFVSLTAIQIGLIDLLSCMGLRPDGIVGHSLGEVACGYADGCLSQEEAVLAAYWRGQCIKEAHLPPGAMAAVGLSWEECKQRCPPGVVPACHNSKDTVTISGPQAPVFEFVEQLRKEGVFAKEVRTGGMAFHSYFMEAIAPPLLQELKKVIREPKPRSARWLSTSIPEAQWHSSLARTSSAEYNVNNLVSPVLFQEALWHVPEHAVVLEIAPHALLQAVLKRGLKPSCTIIPLMKKDHRDNLEFFLAGIGRLHLSGIDANPNALFPPVEFPAPRGTPLISPLIKWDHSLAWDVPAAEDFPNGSGSPSAAIYNIDTSSESPDHYLVDHTLDGRVLFPATGYLSIVWKTLARALGLGVEQLPVVFEDVVLHQATILPKTGTVSLEVRLLEASRAFEVSENGNLVVSGKVYQWDDPDPRLFDHPESPTPNPTEPLFLAQAEVYKELRLRGYDYGPHFQGILEASLEGDSGRLLWKDNWVSFMDTMLQMSILGSAKHGLYLPTRVTAIHIDPATHRQKLYTLQDKAQVADVVVSRWLRVTVAGGVHISGLHTESAPRRQQEQQVPILEKFCFTPHTEEGCLSERAALQEELQLCKGLVQALQTKVTQQGLKMVVPGLDGAQIPRDPSQQELPRLLSAACRLQLNGNLQLELAQVLAQERPKLPEDPLLSGLLDSPALKACLDTAVENMPSLKMKVVEVLAGHGHLYSRIPGLLSPHPLLQLSYTATDRHPQALEAAQAELQQHDVAQGQWDPADPAPSALGSADLLVCNCAVAALGDPASALSNMVAALREGGFLLLHTLLRGHPLGDIVAFLTSTEPQYGQGILSQDAWESLFSRVSLRLVGLKKSFYGSTLFLCRRPTPQDSPIFLPVDDTSFRWVESLKGILADEDSSRPVWLKAINCATSGVVGLVNCLRREPGGNRLRCVLLSNLSSTSHVPEVDPGSAELQKVLQGDLVMNVYRDGAWGAFRHFLLEEDKPEEPTAHAFVSTLTRGDLSSIRWVCSSLRHAQPTCPGAQLCTVYYASLNFRDIMLATGKLSPDAIPGKWTSQDSLLGMEFSGRDASGKRVMGLVPAKGLATSVLLSPDFLWDVPSNWTLEEAASVPVVYSTAYYALVVRGRVRPGETLLIHSGSGGVGQAAIAIALSLGCRVFTTVGSAEKRAYLQARFPQLDSTSFANSRDTSFEQHVLWHTGGKGVDLVLNSLAEEKLQASVRCLATHGRFLEIGKFDLSQNHPLGMAIFLKNVTFHGVLLDAFFNESSADWREVWALVQAGIRDGVVRPLKCTVFHGAQVEDAFRYMAQGKHIGKVVVQVLAEEPEAVLKGAKPKLMSAISKTFCPAHKSYIIAGGLGGFGLELAQWLIQRGVQKLVLTSRSGIRTGYQAKQVRRWRRQGVQVQVSTSNISSLEGARGLIAEAAQLGPVGGVFNLAVVLRDGLLENQTPEFFQDVCKPKYSGTLNLDRVTREACPELDYFVVFSSVSCGRGNAGQSNYGFANSAMERICEKRRHEGLPGLAVQWGAIGDVGILVETMSTNDTIVSGTLPQRMASCLEVLDLFLNQPHMVLSSFVLAEKAAAYRDRDSQRDLVEAVAHILGIRDLAAVNLDSSLADLGLDSLMSVEVRQTLERELNLVLSVREVRQLTLRKLQELSSKADEASELACPTPKEDGLAQQQTQLNLRSLLVNPEGPTLMRLNSVQSSERPLFLVHPIEGSTT

SEQ ID NO: 15 - protein, 2603 aa, Dictyostelium discoideum
MTFNNIKDENNDDIAIIGMGFRFPGGGNNPDQFWNQLSNKMDGISKISQEKWSRSFYEQKYINNEYGGVLKDEEWKNFDPLFFGISPKEAPTIDPQQRLLMTTLWEAFEDANIKPSTLRGSDTAVFIGMMNLDYQRCQFRDISYINPYTVTGSAGSFVSNRLSFSFDLRGPSMTLDTACSSSLNAVYLGCQAIATGDSKMAIVGGVNGIFDPSISMTFSGLNMLGHKGQCRSFDAGADGYIRSEGGGVCILKKYSDAIKDGDRIYCVIKGGSSNVDGYNAKTNITQPSMKAQGENIEIALKKSGVNPSDIYYIEAHGTGTPVGDPIEIEAISRIFKDNHTPDAPLYIGSVKSNIGHLESAAGIASLIKVALSLKNRSLVPNIHFEKPNPLIKFEDWNIRVVTDEIQFPTNKLINMGINCFGLSGSNCHMILSEAPINYDELLKTTNNNSTSSSSNDDKKEYLIPFSANCNISLDKYIEKLISNQSIYKDTILFKDFVKHQTISKSNLIKRKVITASDWDDFLNKRNETTSTSSLTSTISAPASSTPVIYVFTGQGPQWRDMGKALYETESVFKDAIDHCDKLLANYFGYSILQKLRSLESDDSPEIHHPILAQPSIFLIQVGLVALYKSFGISPSIVVGHSFGEVSSALFSGVISLETAVKIVYYRGLAQNLTMGTGRLLSIGIGADAYLEKCALLYPEIEIACYNDPNSIVITGSEQDLLGAKSTLSAEGVFCAFLGTPCSFHSSKQEMIKEKIFKDLSDLPESNVPCVPFFSTITGSQLSHKGFYNVQYIYDNLRMPVEFTKAISNIFNFIEENESYKNAIFLEIGPHPTLGFYIPKCKPSNSTITSKPIIVSPLHKKKEELTQFKLAISTLYCNGVEIDFASGQQLLPTSSSSGGGDISSFKESTNKLPRYQWDFEEYWDEPNQSKMVKRGPSNNLLGHDQFAGNTLMELFIDINKSAHQYLKGHKIKGKYLFPGSGYIDNILRQFNGQDITIFNLEFSNPFFLKDGVQHHLQTSITPTTKGEFKVEFFIKDNRNSTKWTKTSNGRIGLFKHNPKNNKLDIEKLKSQCSFTTLTKSEVYNKLLLLSLPYGPTFQRVESCSIGDGCSFFKLSMSPCSEFDKDFLNPSIIDCAFHGLLVLSEGPQEIVFDRLQDMKFYSSNVPSTRPQFIYAFAKFDKIVGNSTHGSLDIMLEDGTLLISIKNVKCTSLIRLKKQSIKYPSQNVYSHHWQSKDSPLTLIENQLIEEKSSESKINFEKLLNDKLFNDYLIRLLNQSIKSEFIEFDYKTSTVDTLEIDSNNTKLLEKIQSILKTIDSLDQSIDLASLKQVIIEKSSSFKKEINLIEKSIKRIVSLLKGGESEHFSPSNPSSPNDTPRYNSNNCSSKSNNTSSGADDDTNNEETINQLNNEPFNFSNSQFISNQNQLISKTIVNSFDRLINSIEIGEKKLIKIIDLSSIYQNNQLSKLLLLQLNQLLINLSNNNNIEIEYTIPSNTKNIDSIKEETKSISNLLNIKYRSFDLQDDLESNGYLNSNYDLIITSLLLVSTNSIDSNEVLSKLYKLLLPKGQLILMEPPKDVLSFNLLFANDFKQSLEIKSEQEIKSLIRYCGFTKIETNNITQDDEEEQQQPPSILIVQTEKRDIESMSLTFSSDPESLNSSYSNCIFIVSKEQKENPTSYIQEYFDITEVFCDNTTIIEAGDSELLTKTIESGIGKNDIIFFLVSLEELTIENYKQVTMQYTLVNQILLRNNLSTRFALLTYDSQNGGKNYLGSSLIGTFRYFLEFPSLNTFSIDVDKDSIDNLTLFLRLVDLSTIGDRETIVRNNKIFVQKIFKEPKLLSPSNNYEKDTNNLYLNTNSNLDFSFQCKEKLPHGSVEIKVMSTGINYKDNLFYRGLLPQEIFTKGDIYSPPFGLECAGYITRVAPSGVTRFKVGDQVVGFASHSLSSLAITHQDKIVLKPENISFNEAAAVCVVYATSYYSIFHIGAFMADKESILVHSATGGVGLATLNLLKWKRNQLKKHGNSEISNDASIYATVGSKEKVDYLQEKYGDLITAIYNSRDTEYCDEIKQQSAQGGVDLILNTLSGDYLSANFRSLSQVGRIMDLSVTQLVENDSLDFSNFKYHVTYSTIDLERATTYNSKIVRDILTEVFDAISDGSLENIPVKVFPATQVKTAIEYINERVHIGKIVVDFENFEQDILKPALQEKENPIQLNKVKKLEHTCDTLNNTILITGQTGIAVHILKWIISGSVLNSNKSQQQVTDFIILSRSSLKWELENLINQTKHKYGDRFRFHYKSVNIADLNSTRTAIDQVYSSCKNVSPIKSVLHFATVYEYILPEDITQTVIDNTHNPKAVGAINLHNLSIEKDWKLENFILFSSIGAIIGGSKQCAYSSANLVLDSLSNYRKSIGLASTSINWGGLDAGGVAATDKSVASFLEGQGILLVSLSKILGCLDSVFQPSNSHLSNFMLSSFNIDNLLSSAPQMKRKMGHHLTNYKTSSASSDDSLGDSSSTQAKVISTISELLSIHPSKLNLDTRLKDYGIDSLLTVQLKNWIDKEFTKNLFTHLQLSSSSINSIIQRISSKSTSTSTPNPTNTTKQTATTKT

SEQ ID NO: 16 - protein, 2604 aa, Dictyostelium discoideum
MTFNNIKDENNDDIAIIGMGFRFPGGGNNPYQFWNQLSNKMDGISKIPTEKWSRSFYEQKYINNEYGGVLKDEEWKNFDPLFFGISPKEAPIIDPQQRLLMTTLWEAFEDANIKPSTFRGSDTAVFIGMMNTDYQRCQFRDISYVNPYITPGTAGSFISNRLSFSFDLRGPSMTLDTACSSSLNAVYLGCQAIANGDSKMAIVGGVNGIFDPCFSMTFSGLNMLGHKGQCRSFDAGADGYIRSEGGGVCILKKYSDAIKDGDRIYCVIKGGSSNVDGYNAKTNIIQPSMKAQGENIEIALKKSGVNPSDIYYIEAHGTGTPVGDPIEIEAISRIFKDNHTPDAPLYIGSVKSNIGHLESAAGIASLIKVALSLKNRSLVPNIHFEKPNPLIKFEDWNIRVVTDEIQFPINKLINMGINCFGLSGSNCHMILSEAPINYDELLKTTNNNSTSSSSNDDKKEYLIPFSANCNISLDKYIEKLISNQSIYKDTILFKDFVKHQTISKSNLIKRKVITASDWDDFLNKRNETTSTSSLTSTISAPASSTPVIYVFTGQGPQWRDMGKALYETESVFKDAIDHCDKLLANYFGYSILQKLLSLESEDSPEIHHPILAQPSIFLIQVGLVALYKSFGISPSIVVGHSFGEIPSALFSDVISLETAVKIVYYRGLAQNLTMGTGRLLSIGIGADAYLEKCALLYPEIEIACYNDPNSIVITGSEQDLLGAKSTLSAEGVFCAFLGTPCSFHSSKQEMIKEKIFKDLSDLPESNVPCVPFFSTITGSQLSHKGFYNVQYIYDNLRMPVEFTKAISNIFNFIEENESYKNAIFLEIGPHPTLGFYIPKCKPSNSTITSKPIIVSPLHKKKEELTQFKLAISTLYCNGVEIDFASGQQLLPTSSSSGGGDISSFKESTNKLPRYQWDFEEYWDEPNQSKMVKRGPSNNLLGHDQFAGNTLMELFIDIDKSAHQYLKGHKIKGKYLFPGSGYIDNILRQFNGQDITIFNLEFSNPFFLKDGVQHHLQTSITPTTKGEFKVEFFIKDNRNSTKWTKTSNGRIGLFKHNPKNNKLDIEKLKSQCSFTTLTKSEVYNKLLLLSLPYGPTFQRVESCSIGDGCSFFKLSMSPCSEFDKDFLNPSIIDCAFHGLLVLSEGPQEIVFDRLQDMKFYSSNVPSTRPQFIYAFAKFDKIEGNSTHGSLNIILEDGTLLISIKNVKCTSLIRLKKQSIKYPSQNVYSHHWQSKDSPLTLIENQLIEEKSSESKINFEKLLNDKLFNYYLIRLLNQSIKSEFIEFDYKTSTVDTLDIGSKNAKLLEKIQSILNPIDSLDQSIDITSLKQAIIVKSSFKNEIKLVEKSIKRIVSLLKGGESEHFSPSNPSSPNDTPRNNSNNCSSKNNAASSDDADDDTNNEETINQLNNEPFNFSNSQFISNQNQLISKTIVNSFDRLINSIEIGEKKLIKIIDLSSIYQNYQLSKLLLLQLNQLLINLSNNNNIEIEYTIPSNTKNIDSITEETKSISNVLNIKYRSFDLQDDLESNGYLNSNYDLIITSLLLVSTNSIDSNEVLSKLYKLLLPKGQLILMEPPKGVLSFNLLFANDFKQSLEIKSEQEIKSLIIYCGFTKIETNLNTKDDEEQQQPPPPSILIVQAEKRDIESMSLTFSSDPKSLNSSYSNCIFIVSKEQKENPTSYIQEYFDITEFFCQNATIIEADDSELLTKTIESGVGKNDIIFFLVSLEELTIENYKQVTMQYTLVNQILLRNNLSTRFALLTYDSQNGGKNYLGSSLIGTFRYFLEFRSLNIFSIDVDKDSIDNLTLFLRLVDLSTIGDRETIVRNNKIFVQKIFKEPKLLSPSNNYEKNTNNLFLYSNSNLDFSFQSKEKLLHGCVEIKVMSTGINYKDSLFYRGLLPQEVFSKGDIYSPPFGLECAGYITRVAPSGVTRFKVGDQVVGFASHSLSSHVTTHQNKIVLKPENISFNEAAAVCVVYATSYYSIFHIGAFIADKESILVHSATGGVGLASLNLLKWKRNQLKKHGNSEISNDASIYATVGSKEKIDYLQEKYGDLITAIYNSRDTEYCDEIKQQSAQGGVDLILNTLSGDYLSSNFRSLSQVGRIMDLSVTQLVENDSLDFSNFKYHVGYNTIDLDRATKYNSKIIRDILTEVFDAISDGSLENIPVKVFPAIQVKTAIEYINERVHIGKIVVDFENFEQDILKPALQEKENPIQLNKVKKLEHTCDTLNNTILITGQTGIAVHILKWIISGSVLNSNKSQQQVTDFIILSRSSLKWELENLINQTKHKYGDRFRFHYKSVNIADLNSTRTAIDQVYSSCKNVSPIKSVLHFATVYEYILPENITQTVIDNTHNPKAVGAINLHNLSIEKDWKLENFILFSSIGAIIGGSKQCAYSSANLVLDSLSNYRKSIGLASTSINWGGLDAGGVAATDKSVASFLEGQGILLVSLSKILGCLDSVFQPSNSHLSNFMLSSFNIDNLLSSAPQMKRKMDHHLTNYKTSSASSDDSLGDSGSTQAKVISTISELLSIHPSKLNLDTRLKDYGIDSLLTVQLKNWIDKEFTKNLFTHLQLSSSSINSIIQRISSKSTSTSTPNPTNTSKQTATKKT

SEQ ID NO: 17 - protein, 7 aa, Artificial
  synthetic consensus sequence
XAXIGMG

SEQ ID NO: 18 - protein, 4 aa, Artificial
  synthetic consensus sequence
RFPG

SEQ ID NO: 19 - protein, 5 aa, Artificial
  synthetic consensus sequence
FWXNL

SEQ ID NO: 20 - protein, 9 aa, Artificial
  synthetic consensus sequence
IDPQQRLLX

SEQ ID NO: 21 - protein, 8 aa, Artificial
  synthetic consensus sequence
TGVFXGVS

SEQ ID NO: 22 - protein, 25 aa, Artificial
  synthetic consensus sequence
SNRLSXFFDFRGPSITLXTACSSSX

SEQ ID NO: 23 - protein, 7 aa, Artificial
  synthetic consensus sequence
AIVGGXN

SEQ ID NO: 24 - protein, 5 aa, Artificial
  synthetic consensus sequence
LGMLS

SEQ ID NO: 25 - protein, 4 aa, Artificial
  synthetic consensus sequence
RSEG

SEQ ID NO: 26 - protein, 5 aa, Artificial
  synthetic consensus sequence
VLLKK

SEQ ID NO: 27 - protein, 9 aa, Artificial
  synthetic consensus sequence
YIEAHGTGT

SEQ ID NO: 28 - protein, 4 aa, Artificial
  synthetic consensus sequence
VGDP

SEQ ID NO: 29 - protein, 7 aa, Artificial
  synthetic consensus sequence
EPLLIGS

SEQ ID NO: 30 - protein, 8 aa, Artificial
  synthetic consensus sequence
KSNIGHLE

SEQ ID NO: 31 - protein, 5 aa, Artificial
  synthetic consensus sequence
ASGIA

SEQ ID NO: 32 - protein, 4 aa, Artificial
  synthetic consensus sequence
LIKV

SEQ ID NO: 33 - protein, 4 aa, Artificial
  synthetic consensus sequence
LSLK

SEQ ID NO: 34 - protein, 4 aa, Artificial
  synthetic consensus sequence
SPNP

SEQ ID NO: 35 - protein, 11 aa, Artificial
  synthetic consensus sequence
GXNSFGFGGSN

SEQ ID NO: 36 - protein, 7 aa, Artificial
  synthetic consensus sequence
XXFSGQG

SEQ ID NO: 37 - protein, 6 aa, Artificial
  synthetic consensus sequence
QWRGMG

SEQ ID NO: 38 - protein, 5 aa, Artificial
  synthetic consensus sequence
AQPSL

SEQ ID NO: 39 - protein, 7 aa, Artificial
  synthetic consensus sequence
AIQIGLX

SEQ ID NO: 40 - protein, 8 aa, Artificial
  synthetic consensus sequence
VGHSLGEV

SEQ ID NO: 41 - protein, 5 aa, Artificial
  synthetic consensus sequence
XVIAC

SEQ ID NO: 42 - protein, 5 aa, Artificial
  synthetic consensus sequence
KEVRT

SEQ ID NO: 43 - protein, 4 aa, Artificial
  synthetic consensus sequence
AFHS

SEQ ID NO: 44 - protein, 7 aa, Artificial
  synthetic consensus sequence
LEIAPHP

SEQ ID NO: 45 - protein, 4 aa, Artificial
  synthetic consensus sequence
LKSS

SEQ ID NO: 46 - protein, 5 aa, Artificial
  synthetic consensus sequence
YWDEP

SEQ ID NO: 47 - protein, 4 aa, Artificial
  synthetic consensus sequence
ILIK

SEQ ID NO: 48 - protein, 5 aa, Artificial
  synthetic consensus sequence
GQLIL

SEQ ID NO: 49 - protein, 4 aa, Artificial
  synthetic consensus sequence
SLFS

SEQ ID NO: 50 - protein, 9 aa, Artificial
  synthetic consensus sequence
XAASXPXVX

SEQ ID NO: 51 - protein, 7 aa, Artificial
  synthetic consensus sequence
TAYYSLV

SEQ ID NO: 52 - protein, 6 aa, Artificial
  synthetic consensus sequence
ILXHSG

SEQ ID NO: 53 - protein, 6 aa, Artificial
  synthetic consensus sequence
GGVGLA

SEQ ID NO: 54 - protein, 4 aa, Artificial
  synthetic consensus sequence
TVGS

SEQ ID NO: 55 - protein, 5 aa, Artificial
  synthetic consensus sequence
NSRDT

SEQ ID NO: 56 - protein, 12 aa, Artificial
  synthetic consensus sequence
GXDLXLNSLSGX

SEQ ID NO: 57 - protein, 6 aa, Artificial
  synthetic consensus sequence
DAIRYM

SEQ ID NO: 58 - protein, 8 aa, Artificial
  synthetic consensus sequence
HIGKXVXX

SEQ ID NO: 59 - protein, 4 aa, Artificial
  synthetic consensus sequence
STII

SEQ ID NO: 60 - protein, 4 aa, Artificial
  synthetic consensus sequence
SNIS

SEQ ID NO: 61 - protein, 6 aa, Artificial
  synthetic consensus sequence
GXFHLA

SEQ ID NO: 62 - protein, 6 aa, Artificial
  synthetic consensus sequence
NLHRVS

SEQ ID NO: 63 - protein, 5 aa, Artificial
  synthetic consensus sequence
GQSNY

SEQ ID NO: 64 - protein, 4 aa, Artificial
  synthetic consensus sequence
GLPS

SEQ ID NO: 65 - protein, 4 aa, Artificial
  synthetic consensus sequence
LNTV